SEQUENCE LISTING (375 sequences)

SEQ ID NO: 1 - protein, 122 aa, synthetic construct
  REGION 1..122: note = Synthetic: 1D7, VH
EVMLVESGGGLVKPGGSLKLSCVASGFTFSSFAMSWVRQTPEKRLEWVAIISSGGGYIYYADSMTGRFTISRDNAKNTLYLQMSSLRSEDTAMYYCARLGGDYDEFYAKDYWGQGTSVTVSS

SEQ ID NO: 2 - protein, 8 aa, synthetic construct
  REGION 1..8: note = Synthetic: CDRH1-AA
GFTFSSFA

SEQ ID NO: 3 - protein, 8 aa, synthetic construct
  REGION 1..8: note = Synthetic: CDRH2-AA
ISSGGGYI

SEQ ID NO: 4 - protein, 15 aa, synthetic construct
  REGION 1..15: note = Synthetic: CDRH3-AA
ARLGGDYDEFYAKDY

SEQ ID NO: 5 - DNA, 365 bp, synthetic construct
  misc_feature 1..365: note = Synthetic: 1D7, VH
  source 1..365: mol_type = other DNA
gaagttatgttagtcgaaagtggaggcgggctagtgaagcctgggggtagtctgaaactctcctgcgtagcttcaggattcaccttctcgtcatttgcaatgagctgggtcagacagacccccgagaagcggcttgaatgggtggctattataagctccgggggcgggtacatctattatgccgactccatgaccggacgctttacaatctctagggacaatgcaaagaacacgttgtacctgcaaatgtccagcctccgaagcgaggatacagccatgtactattgtgcccgtctgggcggtgactacgatgagttctacgccaaagattattggggccagggcacttcagtgactgtgtcttc

SEQ ID NO: 6 - protein, 106 aa, synthetic construct
  REGION 1..106: note = Synthetic: 1D7, VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTWTFGGGTKLEIK

SEQ ID NO: 7 - protein, 6 aa, synthetic construct
  REGION 1..6: note = Synthetic: CDRL1-AA
EDIYNR

SEQ ID NO: 8 - skipped

SEQ ID NO: 9 - protein, 8 aa, synthetic construct
  REGION 1..8: note = Synthetic: CDR:3-AA
QQYWSTWT

SEQ ID NO: 10 - DNA, 316 bp, synthetic construct
  misc_feature 1..316: note = Synthetic: 1D7, VL
  source 1..316: mol_type = other DNA
acatacagatgacccaatctagcagttcgttcagcgtgtcacttggtgatcgcgtgactatcacatgtaaggctagtgaggacatttataaccgactggcctggtatcagcagaagcccggcaatgcaccacggctgttaatctctggagctacttccttggaaacaggagtcccttccaggttctccgggagcggcagcgggaaggactacaccctgtctatcacatcccttcagacagaggatgttgccacctactattgccagcaatactggtcaacttggaccttcggaggcggtacgaaactcgaaattaa

SEQ ID NO: 11 - protein, 119 aa, synthetic construct
  REGION 1..119: note = Synthetic: 8G4, VH
QIQLVQSGPELKMPGETVKISCKASGYTFTNFGMNWVKQAPGKALKWMGWINTNTGKPTYFEEFKGRFAFSLETSASTAYLQINNLKNEDTATYFCARRWDRSAWFAYWGQGTLVTVSA

SEQ ID NO: 12 - protein, 8 aa, synthetic construct
  REGION 1..8: note = Synthetic: CDRH1-AA
GYTFTNFG

SEQ ID NO: 13 - protein, 8 aa, synthetic construct
  REGION 1..8: note = Synthetic: CDRH2-AA
INTNTGKP

SEQ ID NO: 14 - protein, 12 aa, synthetic construct
  REGION 1..12: note = Synthetic: CDRH3-AA
ARRWDRSAWFAY

SEQ ID NO: 15 - DNA, 357 bp, synthetic construct
  misc_feature 1..357: note = Synthetic: 8G4, VH
  source 1..357: mol_type = other DNA
cagatccagttggtgcagtctggacctgagctgaagatgcctggagagacagtcaagatctcctgcaaggcttctggatataccttcacaaactttggaatgaactgggtgaagcaggctccaggaaaggctttaaagtggatgggctggataaacaccaacactggaaagccaacatattttgaagagttcaagggacggtttgccttctctttggaaacctctgccagcactgcctatttgcagatcaataatctcaaaaatgaggacacggctacatatttctgtgcaagacgctgggaccggtccgcctggtttgcttactggggccaagggactctggtcactgtctctgca

SEQ ID NO: 16 - protein, 109 aa, synthetic construct
  REGION 1..109: note = Synthetic: 8G4, VL
QAVVTQESALTTSPGETVTLTCRSSTGAVTTSNYANWVQEKPDHLFTGLIGGTNDRSPGVPARFSGSLIGDKAALTITGAQTEDEAIYFCALWYSNHWVFGGGTKLTVL

SEQ ID NO: 17 - protein, 9 aa, synthetic construct
  REGION 1..9: note = Synthetic: CDRL1-AA
TGAVTTSNY

SEQ ID NO: 18 - skipped

SEQ ID NO: 19 - protein, 9 aa, synthetic construct
  REGION 1..9: note = Synthetic: CDR:3-AA
ALWYSNHWV

SEQ ID NO: 20 - DNA, 327 bp, synthetic construct
  misc_feature 1..327: note = Synthetic: 8G4, VL
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacgaccgatctccaggtgttcctgccagattctcaggctccctgattggagacaaggctgccctcaccatcacaggggcacagactgaggatgaggcaatatatttctgtgctctctggtacagcaaccattgggtgttcggtggaggaaccaaactgactgtccta

SEQ ID NO: 21 - protein, 114 aa, synthetic construct
  REGION 1..114: note = Synthetic: 21G6, VH
EVQLEESGPGLVKPSQSLSLTCTVTGYSITSDYAWNWIRQFPGNKLEWMGYISYSGSTIYNPSLKSRISITRDTSKNQFFLHLNSVTTEDTATYYCATLGLDYWGQGTTLTVSS

SEQ ID NO: 22 - protein, 9 aa, synthetic construct
  REGION 1..9: note = Synthetic: CDRH1-AA
GYSITSDYA

SEQ ID NO: 23 - protein, 7 aa, synthetic construct
  REGION 1..7: note = Synthetic: CDRH2-AA
ISYSGST

SEQ ID NO: 24 - protein, 7 aa, synthetic construct
  REGION 1..7: note = Synthetic: CDRH3-AA
ATLGLDY

SEQ ID NO: 25 - DNA, 342 bp, synthetic construct
  misc_feature 1..342: note = Synthetic: 21G6, VH
  source 1..342: mol_type = other DNA
gaggtgcagctggaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacagtggtagcactatctacaacccatctctcaaaagtcgaatctctatcactcgagacacatccaagaaccagttcttcctgcacttgaattctgtgactactgaggacacagccacatattactgtgcaactctgggacttgactactggggccaaggcaccactctcacagtctcctca

SEQ ID NO: 26 - protein, 112 aa, synthetic construct
  REGION 1..112: note = Synthetic: 21G6, VL
DFVMTQSPSSLAVSVGEKVTMSCKSSQSLLYSSNQKNYLAWYQQKPGQSPKLLIYWASTRGSGVPDRFTGSGSGTDFTLTISSVKAEDLAVYYCQQYHSYRTFGGGTKLEIK

SEQ ID NO: 27 - protein, 12 aa, synthetic construct
  REGION 1..12: note = Synthetic: CDRL1-AA
QSLLYSSNQKNY

SEQ ID NO: 28 - skipped

SEQ ID NO: 29 - protein, 8 aa, synthetic construct
  REGION 1..8: note = Synthetic: CDR:3-AA
QQYHSYRT

SEQ ID NO: 30 - DNA, 336 bp, synthetic construct
  misc_feature 1..336: note = Synthetic: 21G6, VL
  source 1..336: mol_type = other DNA
gattttgtgatgacgcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactaggggatctggggtccctgatcgcttcacaggcagtggatctgggacagatttcactctcaccatcagcagtgtgaaggctgaagacctggcagtttattactgtcagcaatatcatagctatcggacgttcggtggaggcaccaagctggaaatcaag

SEQ ID NO: 31 - protein, 117 aa, synthetic construct
  REGION 1..117: note = Synthetic: M6I-02-A03, VH
EVQLLETGGGLVRPGGSRGLSCEGSGFTFSGFWMSWVRQTPGKTLEWIGDINSDGSAVNYAPSIKDRFTIFRDNDKSTLYLQMSNVRSEDTATYFCMRGTGWYFDVWGAGTTVTVSS

SEQ ID NO: 32 - protein, 8 aa, synthetic construct
  REGION 1..8: note = Synthetic: CDRH1-AA
GFTFSGFW

SEQ ID NO: 33 - protein, 8 aa, synthetic construct
  REGION 1..8: note = Synthetic: CDRH2-AA
INSDGSAV

SEQ ID NO: 34 - protein, 10 aa, synthetic construct
  REGION 1..10: note = Synthetic: CDRH3-AA
MRGTGWYFDV

SEQ ID NO: 35 - DNA, 352 bp, synthetic construct
  misc_feature 1..352: note = Synthetic: M6I-02-A03, VH
  source 1..352: mol_type = other DNA
gaagtgcagctgttggagactggaggaggcttggtgcgacctggggggtcacggggactctcttgtgaaggctcagggtttacttttagtggcttctggatgagctgggttcgacagacacctgggaagaccctggagtggattggagacattaattctgatggcagtgcagtaaactacgcaccatccataaaggatcgattcactatcttcagagacaatgacaagagcaccctgtacctgcagatgagcaatgtgcgatctgaggacacagccacgtatttctgtatgagggggactgggtggtacttcgatgtctggggcgcagggaccacggtcaccgtctcctcgg

SEQ ID NO: 36 - protein, 112 aa, synthetic construct
  REGION 1..112: note = Synthetic: M6I-02-A03, VL
DIVMTQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIK

SEQ ID NO: 37 - protein, 12 aa, synthetic construct
  REGION 1..12: note = Synthetic: CDRL1-AA
QSLLNSRTRKNY

SEQ ID NO: 38 - skipped

SEQ ID NO: 39 - protein, 8 aa, synthetic construct
  REGION 1..8: note = Synthetic: CDR:3-AA
KQSYNLYT

SEQ ID NO: 40 - DNA, 337 bp, synthetic construct
  misc_feature 1..337: note = Synthetic: M6I-02-A03, VL
  source 1..337: mol_type = other DNA
gacattgtgatgacacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtacacgttcggaggggggaccaagctggaaatcaaac

SEQ ID NO: 41 - protein, 121 aa, synthetic construct
  REGION 1..121: note = Synthetic: M6I-02-C02, VH
DVMLVESGGGLVQPGASLRLSCATLGFTFTDYYMSWVRQPPGKALEWLGFIRDKANGYTTEYSASVKGRFTISRDNSQSILYLQMNTLRAEDSATYYCARDLFGLYYFDYWGQGTTLTVSS

SEQ ID NO: 42 - protein, 8 aa, synthetic construct
  REGION 1..8: note = Synthetic: CDRH1-AA
GFTFTDYY

SEQ ID NO: 43 - protein, 10 aa, synthetic construct
  REGION 1..10: note = Synthetic: CDRH2-AA
IRDKANGYTT

SEQ ID NO: 44 - protein, 12 aa, synthetic construct
  REGION 1..12: note = Synthetic: CDRH3-AA
ARDLFGLYYFDY

SEQ ID NO: 45 - DNA, 364 bp, synthetic construct
  misc_feature 1..364: note = Synthetic: M6I-02-C02, VH
  source 1..364: mol_type = other DNA
gacgtgatgctggtggagtctggaggaggcttggtacagcctggggcttctctgagactctcctgtgcaactttggggttcaccttcactgactactacatgagctgggtccgccagcctccaggaaaggcacttgagtggttgggttttattagagacaaagctaatggttacacaacagagtacagtgcatctgtgaagggtcggttcaccatctccagagataattcccaaagcatcctctatcttcaaatgaacaccctgagagctgaggacagtgccacttattactgtgcaagagatctctttggcctgtactactttgactactggggccagggcaccactctcacagtctcctcgg

SEQ ID NO: 46 - protein, 106 aa, synthetic construct
  REGION 1..106: note = Synthetic: M6I-02-C02, VL
DIVLTQSPAIMSASPGEKVTMTCSASSSVSYMHWYQQKSGTSPKRWIYDTSKLASGVPARFSGSGSGTSYSLTISSMEAEDAATYFCHQWTTYPYTFGGGTKLEIK

SEQ ID NO: 47 - protein, 5 aa, synthetic construct
  REGION 1..5: note = Synthetic: CDRL1-AA
SSVSY

SEQ ID NO: 48 - skipped

SEQ ID NO: 49 - protein, 9 aa, synthetic construct
  REGION 1..9: note = Synthetic: CDR:3-AA
HQWTTYPYT

SEQ ID NO: 50 - DNA, 319 bp, synthetic construct
  misc_feature 1..319: note = Synthetic: M6I-02-C02, VL
  source 1..319: mol_type = other DNA
gacattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttattctctcacaatcagcagcatggaggctgaagatgctgccacttatttctgccatcagtggactacttacccatacacgttcggaggggggaccaagctggaaataaaac

SEQ ID NO: 51 - protein, 122 aa, synthetic construct
  REGION 1..122: note = Synthetic: M6I-02-C06, VH
DVQLVESGGGLVKPGGSLKLSCAASGFTFSNYAMSWVRQTPEKRLEWVASISSRGYTYYADSVKADSVKGRFTISRDNARNILYLQMSSLRSEDTAMFYCARELRLRYFDVWGAGTTVTVSS

SEQ ID NO: 52 - protein, 8 aa, synthetic construct
  REGION 1..8: note = Synthetic: CDRH1-AA
GFTFSNYA

SEQ ID NO: 53 - protein, 7 aa, synthetic construct
  REGION 1..7: note = Synthetic: CDRH2-AA
ISSRGYT

SEQ ID NO: 54 - protein, 16 aa, synthetic construct
  REGION 1..16: note = Synthetic: CDRH3-AA
AMFYCARELRLRYFDV

SEQ ID NO: 55 - DNA, 367 bp, synthetic construct
  misc_feature 1..367: note = Synthetic: M6I-02-C06, VH
  source 1..367: mol_type = other DNA
gacgtgcagctggtggagtctgggggaggcttagtgaagcctggagggtccctgaaactctcctgtgcagcctctggattcactttcagtaactatgccatgtcttgggttcgccagactccagagaagaggctggagtgggtcgcatccattagtagtcgtggttacacctactatgcagacagtgtgaaggcagacagtgtgaagggccgattcaccatctccagagataatgccaggaacatcctgtacctgcaaatgagcagtctgaggtctgaggacacggccatgttttactgtgcgagagaactacggctacggtacttcgatgtctggggcgcagggaccactgtcaccgtctcctcgg

SEQ ID NO: 56 - protein, 112 aa, synthetic construct
  REGION 1..112: note = Synthetic: M6I-02-C06, VL
DVVLTQTPLTLSVTIGQPASISCKSSQSLLDSDGKTYLNWLLQRPGQSPKRLIHLVSKLDSGVPDRFTGSGSGTDFTLKISRVEAEDLGVYYCWQGTHFPRTFGGGTKLEIK

SEQ ID NO: 57 - protein, 11 aa, synthetic construct
  REGION 1..11: note = Synthetic: CDRL1-AA
QSLLDSDGKTY

SEQ ID NO: 58 - skipped

SEQ ID NO: 59 - protein, 9 aa, synthetic construct
  REGION 1..9: note = Synthetic: CDR:3-AA
WQGTHFPRT

SEQ ID NO: 60 - DNA, 337 bp, synthetic construct
  misc_feature 1..337: note = Synthetic: M6I-02-C06, VL
  source 1..337: mol_type = other DNA
gatgttgtgctgacccagactccactcactttgtcggttaccattggacaaccagcctccatctcctgcaagtcaagtcagagcctcttagatagtgatggaaagacatatttgaattggttgttacagaggccaggccagtctccaaagcgcctaatccatctggtgtctaaactggactctggagtccctgacaggtttactggcagtggatcagggacagatttcacactgaaaatcagcagagtggaggctgaggatttgggagtttattattgctggcaaggtacacattttcctcggacgttcggtggaggcaccaagctggaaatcaaac

SEQ ID NO: 61 - protein, 115 aa, synthetic construct
  REGION 1..115: note = Synthetic: M6I-02-D03, VH
QIQLVQSGPELKKPGETVRISCKASGYTFTTAGMQWVQKMPGKGLKWIGWINTHSGVPKYAEDFKGRFAFSLETSASTAYLQISNLKNEDTATYFCARWQAFDYWGQGTTVTVSS

SEQ ID NO: 62 - protein, 8 aa, synthetic construct
  REGION 1..8: note = Synthetic: CDRH1-AA
GYTFTTAG

SEQ ID NO: 63 - protein, 8 aa, synthetic construct
  REGION 1..8: note = Synthetic: CDRH2-AA
INTHSGVP

SEQ ID NO: 64 - protein, 8 aa, synthetic construct
  REGION 1..8: note = Synthetic: CDRH3-AA
ARWQAFDY

SEQ ID NO: 65 - DNA, 346 bp, synthetic construct
  misc_feature 1..346: note = Synthetic: M6I-02-D03, VH
  source 1..346: mol_type = other DNA
cagatccagttggtgcagtctggacctgagctgaagaagcctggagagacagtcaggatctcctgcaaggcttctgggtataccttcacaactgctggaatgcagtgggtgcaaaagatgccaggaaagggtttgaagtggattggctggataaacacccactctggagtgccaaaatatgcagaagacttcaagggacggtttgccttctctttggaaacctctgccagcactgcatatttacagataagcaacctcaaaaatgaggacacggctacgtatttctgtgcgagatggcaagcttttgactactggggccaaggcaccactgtcaccgtctcctcgg

SEQ ID NO: 66 - protein, 107 aa, synthetic construct
  REGION 1..107: note = Synthetic: M6I-02-D03, VL
DIVMTQSQKFMSTSVGDRVSVTCKASQNVGTNVAWYQQKPGQSPKALIYSASYRYSGVPDRFTGSGSGTDFTLTISNVQSEDLADYFCQQYNSYPLTFGGGTKLEIK

SEQ ID NO: 67 - protein, 6 aa, synthetic construct
  REGION 1..6: note = Synthetic: CDRL1-AA
QNVGTN

SEQ ID NO: 68 - skipped

SEQ ID NO: 69 - protein, 9 aa, synthetic construct
  REGION 1..9: note = Synthetic: CDR:3-AA
QQYNSYPLT

SEQ ID NO: 70 - DNA, 322 bp, synthetic construct
  misc_feature 1..322: note = Synthetic: M6I-02-D03, VL
  source 1..322: mol_type = other DNA
gatattgtgatgacccagtctcaaaaattcatgtccacatcagtaggagacagggtcagtgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccattagcaatgtgcagtctgaagacttggcagactatttctgtcagcaatataacagctatcctctcacgttcggaggggggaccaagctggaaataaaac

SEQ ID NO: 71 - protein, 116 aa, synthetic construct
  REGION 1..116: note = Synthetic: M6I-02-D10, VH
DVKLVESGGGLVQPGGSRKLSCAASGFAFNNFGIYWVRQAPEKGLEWVAYISRGNTIYYADTVKGRFTISRDNPKNTLFLQMTSLRSEDTAMYYCGRSYGNYVNYWGLGTTLTVSS

SEQ ID NO: 72 - protein, 8 aa, synthetic construct
  REGION 1..8: note = Synthetic: CDRH1-AA
GFAFNNFG

SEQ ID NO: 73 - protein, 7 aa, synthetic construct
  REGION 1..7: note = Synthetic: CDRH2-AA
ISRGNTI

SEQ ID NO: 74 - protein, 10 aa, synthetic construct
  REGION 1..10: note = Synthetic: CDRH3-AA
GRSYGNYVNY

SEQ ID NO: 75 - DNA, 349 bp, synthetic construct
  misc_feature 1..349: note = Synthetic: M6I-02-D10, VH
  source 1..349: mol_type = other DNA
gacgtgaagctggtggagtctgggggaggcttagtgcagcctggagggtcccggaaactctcctgtgcagcctctggattcgctttcaataattttggaatatactgggttcgtcaggctccagagaaggggctggagtgggtcgcatacattagtcgtggtaataccatctactatgcagacacagtgaagggccgattcaccatctccagagacaatcccaagaacaccctgttcctgcaaatgaccagtctaaggtctgaggacacggccatgtattactgtggaagatcgtatggtaactacgtgaactactggggcctaggcaccactctcacagtctcctcgg

SEQ ID NO: 76 - protein, 112 aa, synthetic construct
  REGION 1..112: note = Synthetic: M6I-02-D10, VL
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIK

SEQ ID NO: 77 - protein, 11 aa, synthetic construct
  REGION 1..11: note = Synthetic: CDRL1-AA
QSLLDSDGKTY

SEQ ID NO: 78 - skipped

SEQ ID NO: 79 - protein, 9 aa, synthetic construct
  REGION 1..9: note = Synthetic: CDR:3-AA
WQGTHFPYT

SEQ ID NO: 80 - DNA, 337 bp, synthetic construct
  misc_feature 1..337: note = Synthetic: M6I-02-D10, VL
  source 1..337: mol_type = other DNA
gatattgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaagctggaaataaaac

SEQ ID NO: 81 - protein, 120 aa, synthetic construct
  REGION 1..120: note = Synthetic: M6I-02-E01, VH
EVMLVESGGGLVQPGGSLRLSCATSGFTFTDYYMSWVRQPPGKALEWLGFIRNKPNGYTTEYSASVKGRFTISRDNSQSILYLQMNTLRAEDSATYYCARDNYGLYFDYWGQGTTLTVSS

SEQ ID NO: 82 - protein, 8 aa, synthetic construct
  REGION 1..8: note = Synthetic: CDRH1-AA
GFTFTDYY

SEQ ID NO: 83 - protein, 10 aa, synthetic construct
  REGION 1..10: note = Synthetic: CDRH2-AA
IRNKPNGYTT

SEQ ID NO: 84 - protein, 11 aa, synthetic construct
  REGION 1..11: note = Synthetic: CDRH3-AA
ARDNYGLYFDY

SEQ ID NO: 85 - DNA, 361 bp, synthetic construct
  misc_feature 1..361: note = Synthetic: M6I-02-E01, VH
  source 1..361: mol_type = other DNA
gaggtgatgctggtggagtctggaggaggcttggtacagcctgggggttctctgagactctcctgtgcaacttctgggttcaccttcactgattactacatgagttgggtccgccagcctccaggaaaggcacttgagtggttgggttttattagaaacaaacctaatggttacacaacagagtacagtgcatctgtgaagggtcggttcaccatctccagagataattcccaaagcatcctctatcttcaaatgaacaccctgagagctgaggacagtgccacttattactgtgcaagagataactacggcctctactttgactactggggccaaggcaccactctcacagtctcctcgg

SEQ ID NO: 86 - protein, 106 aa, synthetic construct
  REGION 1..106: note = Synthetic: M6I-02-E01, VL
DIVLTQSPAIMSASPGEKVTMTCSASSSISYMHWYQQKPGTSPKRWIYDTSKLASGVPARFSGSGSGTSYSLTISSMEAEDAATYYCHQRSSYPWTFGGGTKLEIK

SEQ ID NO: 87 - protein, 5 aa, synthetic construct
  REGION 1..5: note = Synthetic: CDRL1-AA
SSISY

SEQ ID NO: 88 - skipped

SEQ ID NO: 89 - protein, 9 aa, synthetic construct
  REGION 1..9: note = Synthetic: CDR:3-AA
HQRSSYPWT

SEQ ID NO: 90 - DNA, 319 bp, synthetic construct
  misc_feature 1..319: note = Synthetic: M6I-02-E01, VL
  source 1..319: mol_type = other DNA
gacattgttctcacccagtctccagcaatcatgtctgcatctccaggggagaaggtcaccatgacctgcagtgccagctcaagtataagttacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacccatggacgttcggtggaggcaccaagctggaaatcaaac

SEQ ID NO: 91 - protein, 120 aa, synthetic construct
  REGION 1..120: note = Synthetic: M6I-04-A02, VH
EVRLQQSGPELVKPGASVKISCKTSGYTFTEYTMHWVKQSHGKSLEWIGGIVPNNGGTNYNQKFKVKATLTVDKSSSTAYMELRSLTSEDSAVYYCARRDYYGSSHFDYWGQGTTLTVSS

SEQ ID NO: 92 - protein, 8 aa, synthetic construct
  REGION 1..8: note = Synthetic: CDRH1-AA
GYTFTEYT

SEQ ID NO: 93 - protein, 8 aa, synthetic construct
  REGION 1..8: note = Synthetic: CDRH2-AA
IVPNNGGT

SEQ ID NO: 94 - protein, 13 aa, synthetic construct
  REGION 1..13: note = Synthetic: CDRH3-AA
ARRDYYGSSHFDY

SEQ ID NO: 95 - DNA, 361 bp, synthetic construct
  misc_feature 1..361: note = Synthetic: M6I-04-A02, VH
  source 1..361: mol_type = other DNA
gaggttcggctgcaacagtctggacctgagctggtgaagcctggggcttcagtgaagatatcctgcaagacttctggatacacattcactgaatacaccatgcactgggtgaagcagagccatggaaagagccttgagtggattggaggtattgttcctaacaatggtggtactaactacaaccagaagttcaaggtcaaggccactttgactgtagacaagtcctccagcacagcctacatggagctccgcagcctgacatctgaggattctgcagtctattactgtgcaaggagggattactacggtagtagccactttgactactggggccaaggcaccactctcacagtctcctcgg

SEQ ID NO: 96 - protein, 112 aa, synthetic construct
  REGION 1..112: note = Synthetic: M6I-04-A02, VL
DILMTQSPSSLAVSVGEKVTMSCKSSQSLLNSRTRKNYLAWYQQKPGQSPKLLIYWASTRESGVPDRFTGSGSGTDFTLTISNVQAEDLAVYYCKQSYNLYTFGGGTKLEIK

SEQ ID NO: 97 - protein, 12 aa, synthetic construct
  REGION 1..12: note = Synthetic: CDRL1-AA
QSLLNSRTRKNY

SEQ ID NO: 98 - skipped

SEQ ID NO: 99 - protein, 8 aa, synthetic construct
  REGION 1..8: note = Synthetic: CDR:3-AA
KQSYNLYT

SEQ ID NO: 100 - DNA, 337 bp, synthetic construct
  misc_feature 1..337: note = Synthetic: M6I-04-A02, VL
  source 1..337: mol_type = other DNA
gacattctgatgacccagtctccatcctccctagctgtgtcagttggagagaaggtcactatgagctgcaaatccagtcagagtctgctcaacagtagaacccgaaagaactacttggcctggtaccagcagaaaccagggcagtctcctaaactgctgatttactgggcatccactagggaatctggggtccctgatcgcttcacaggcagtggatctgggacagatttcactctcaccattagcaatgtgcaggctgaagacctggcagtttattactgcaagcaatcttataatctgtacacgttcggaggggggaccaagctggaaatcaaac

SEQ ID NO: 101 - protein, 119 aa, synthetic construct
  REGION 1..119: note = Synthetic: M6I-04-F11, VH
QVQLQQPGAELVKPGASVKLSCKASGYTFTNSYIYWVKQRPGQGLEWIGGIDPNNDDTNFNEEFKSKATLTVDKSSSTAYMQLSSLTSEDSAVYYCTRLLRYQAWFAYWGQGTLVTVSS

SEQ ID NO: 102 - protein, 8 aa, synthetic construct
  REGION 1..8: note = Synthetic: CDRH1-AA
GYTFTNSY

SEQ ID NO: 103 - protein, 8 aa, synthetic construct
  REGION 1..8: note = Synthetic: CDRH2-AA
IDPNNDDT

SEQ ID NO: 104 - protein, 12 aa, synthetic construct
  REGION 1..12: note = Synthetic: CDRH3-AA
TRLLRYQAWFAY

SEQ ID NO: 105 - DNA, 358 bp, synthetic construct
  misc_feature 1..358: note = Synthetic: M6I-04-F11, VH
  source 1..358: mol_type = other DNA
caggtccaactgcagcagcctggggctgaactggtgaagcctggggcttcagtgaagttgtcctgcaaggcttctggctacaccttcaccaactcctatatctactgggtgaagcagaggcctggacaaggccttgagtggattggggggattgatcctaacaatgatgatactaacttcaatgaggagttcaagagcaaggccacactgactgtggacaaatcctccagcacagcctacatgcaactcagcagcctgacatctgaggactctgcggtctattactgtacaagattactacgctaccaggcctggtttgcttactggggccaggggactctggtcaccgtctcctcgg

SEQ ID NO: 106 - protein, 111 aa, synthetic construct
  REGION 1..111: note = Synthetic: M6I-04-F11, VL
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 107 - protein, 10 aa, synthetic construct
  REGION 1..10: note = Synthetic: CDRL1-AA
ESVDSYGNSF

SEQ ID NO: 108 - skipped

SEQ ID NO: 109 - protein, 9 aa, synthetic construct
  REGION 1..9: note = Synthetic: CDR:3-AA
QQNNEDPWT

SEQ ID NO: 110 - DNA, 334 bp, synthetic construct
  misc_feature 1..334: note = Synthetic: M6I-04-F11, VL
  source 1..334: mol_type = other DNA
gacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaataaaac

SEQ ID NO: 111 - protein, 120 aa, synthetic construct; identical to SEQ ID NO: 91
  REGION 1..120: note = Synthetic: M6I-04-H05, VH

SEQ ID NO: 112 - protein, 8 aa, synthetic construct
  REGION 1..8: note = Synthetic: CDRH1-AA
GYTFTEYT

SEQ ID NO: 113 - protein, 8 aa, synthetic construct
  REGION 1..8: note = Synthetic: CDRH2-AA
IVPNNGGT

SEQ ID NO: 114 - protein, 13 aa, synthetic construct
  REGION 1..13: note = Synthetic: CDRH3-AA
ARRDYYGSSHFDY

SEQ ID NO: 115 - DNA, 361 bp, synthetic construct; identical to SEQ ID NO: 95
  misc_feature 1..361: note = Synthetic: M6I-04-H05, VH
  source 1..361: mol_type = other DNA

SEQ ID NO: 116 - protein, 112 aa, synthetic construct
  REGION 1..112: note = Synthetic: M6I-04-H05, VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 117 - protein, 11 aa, synthetic construct
  REGION 1..11: note = Synthetic: CDRL1-AA
QSLVHSNGNTY

SEQ ID NO: 118 - skipped

SEQ ID NO: 119 - protein, 9 aa, synthetic construct
  REGION 1..9: note = Synthetic: CDR:3-AA
SQSTHVPWT

SEQ ID NO: 120 - DNA, 337 bp, synthetic construct
  misc_feature 1..337: note = Synthetic: M6I-04-H05, VL
  source 1..337: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaataaaac

SEQ ID NO: 121 - protein, 118 aa, synthetic construct
  REGION 1..118: note = Synthetic: M6I-07-A03, VH
EVQLLESGGGLVQPGGSLRLSCAASGFTLSSYWMHWVRQAPGKGLEWVSAISPAGGGTYYADSVKGRFTISRDNSKNTLYLQMNSLRAEDTAVYYCASPLYPYGLDIWGQGTLVTVSS

SEQ ID NO: 122 - protein, 8 aa, synthetic construct
  REGION 1..8: note = Synthetic: CDRH1-AA
GFTLSSYW

SEQ ID NO: 123 - protein, 8 aa, synthetic construct
  REGION 1..8: note = Synthetic: CDRH2-AA
ISPAGGGT

SEQ ID NO: 124 - protein, 11 aa, synthetic construct
  REGION 1..11: note = Synthetic: CDRH3-AA
ASPLYPYGLDI

SEQ ID NO: 125 - DNA, 355 bp, synthetic construct
  misc_feature 1..355: note = Synthetic: M6I-07-A03, VH
  source 1..355: mol_type = other DNA
gaggtgcagctcttagaaagcggaggtggcttggttcagcctggcggtagtctaagactgtcctgtgcagcttctggctttaccctgagcagctattggatgcattgggtccgtcaagcaccaggtaagggtctggaatgggtgtctgcaatctctccggcgggtggcggcacctactatgccgatagcgtgaaaggacgcttcaccatcagcagggacaattccaagaacacgctgtacctgcaaatgaacagcctgcgagccgaggatacggccgtgtattactgtgccagtcctctgtacccatacggcctggatatttggggccagggaaccctggtcaccgtctcctcag

SEQ ID NO: 126 - protein, 107 aa, synthetic construct
  REGION 1..107: note = Synthetic: M6I-07-A03, VL
DIQMTQSPSSLSASVGDRVTITCRASQDISIYLNWYQQKPGKAPKLLIYHASNLQSGVPSRFSGSGSGTDFTLTISSLQPEDFATYYCQQNYGTPYTFGGGTKVEIK

SEQ ID NO: 127 - protein, 6 aa, synthetic construct
  REGION 1..6: note = Synthetic: CDRL1-AA
QDISIY

SEQ ID NO: 128 - skipped

SEQ ID NO: 129 - protein, 9 aa, synthetic construct
  REGION 1..9: note = Synthetic: CDR:3-AA
QQNYGTPYT

SEQ ID NO: 130 - DNA, 322 bp, synthetic construct
  misc_feature 1..322: note = Synthetic: M6I-07-A03, VL
  source 1..322: mol_type = other DNA
gatattcagatgacccagagcccatcctccctgtcagcttctgtaggagaccgcgttaccatcacctgtcgtgcctcccaggatattagcatctacctgaactggtatcagcagaagcctggtaaggcaccgaagctgctgatctatcatgcctccaacctgcaatctggcgtcccgtcacgctttagtggctctggttctgggaccgacttcactctcaccattagcagtctgcaaccggaggacttcgcgacatactattgccagcagaactacggcactccgtatacctttggaggcggtaccaaggtggagatcaaag

SEQ ID NO: 131 - protein, 120 aa, synthetic construct
  REGION 1..120: note = Synthetic: M6I-07-A05, VH
EVQLLESGGGLVQPGGSLRLSCAASGFTFSNYAMGWVRQAPGKGLEWVSVISSGGGGGTSYADSVKGRFTISRDNSKNTLYLQMNSLRAEDTAVYYCASPPRDYEGMDVWGQGTLVTVSS

SEQ ID NO: 132 - protein, 8 aa, synthetic construct
  REGION 1..8: note = Synthetic: CDRH1-AA
GFTFSNYA

SEQ ID NO: 133 - protein, 7 aa, synthetic construct
  REGION 1..7: note = Synthetic: CDRH2-AA
ISSGGGG

SEQ ID NO: 134 - protein, 14 aa, synthetic construct
  REGION 1..14: note = Synthetic: CDRH3-AA
YCASPPRDYEGMDV

SEQ ID NO: 135 - DNA, 361 bp, synthetic construct
  misc_feature 1..361: note = Synthetic: M6I-07-A05, VH
  source 1..361: mol_type = other DNA
gaggtgcagctcttagaaagcggaggtggcttggttcagcctggcggtagtctaagactgtcctgtgcagcttctggcttcacctttagcaactatgcgatgggttgggtccgtcaagcaccaggtaagggtctggaatgggtgtctgtcatctcttccggtggcggtggtggaacctcttatgccgatagcgtgaaaggacgcttcaccatcagcagggacaattccaagaacacgctgtacctgcaaatgaacagcctgcgagccgaggatacggccgtgtattactgtgcttcacctcctcgagactacgagggcatggatgtttggggccagggaaccctggtcaccgtctcctcag

SEQ ID NO: 136 - protein, 107 aa, synthetic construct
  REGION 1..107: note = Synthetic: M6I-07-A05, VL
DIQMTQSPSSLSASVGDRVTITCRASENIGDYLSWYQQKPGKAPKLLIYAASSLQSGVPSRFSGSGSGTDFTLTISSLQPEDFATYYCQQNYDFPLTFGGGTKVEIK

SEQ ID NO: 137 - protein, 6 aa, synthetic construct
  REGION 1..6: note = Synthetic: CDRL1-AA
ENIGDY

SEQ ID NO: 138 - skipped

SEQ ID NO: 139 - protein, 9 aa, synthetic construct
  REGION 1..9: note = Synthetic: CDR:3-AA
QQNYDFPLT

SEQ ID NO: 140 - DNA, 322 bp, synthetic construct
  misc_feature 1..322: note = Synthetic: M6I-07-A05, VL
  source 1..322: mol_type = other DNA
gatattcagatgacccagagcccatcctccctgtcagcttctgtaggagaccgcgttaccatcacctgtcgtgcctccgagaacattggcgattacctgagctggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagaactatgatttcccgctgacttttggaggcggtaccaaggtggagatcaaag

SEQ ID NO: 141 - protein, 120 aa, synthetic construct; identical to SEQ ID NO: 131
  REGION 1..120: note = Synthetic: M6I-07-B05, VH

SEQ ID NO: 142 - protein, 8 aa, synthetic construct
  REGION 1..8: note = Synthetic: CDRH1-AA
GFTFSNYA

SEQ ID NO: 143 - protein, 7 aa, synthetic construct
  REGION 1..7: note = Synthetic: CDRH2-AA
ISSGGGG

SEQ ID NO: 144 - protein, 14 aa, synthetic construct
  REGION 1..14: note = Synthetic: CDRH3-AA
YCASPPRDYEGMDV

SEQ ID NO: 145 - DNA, 361 bp, synthetic construct; identical to SEQ ID NO: 135
  misc_feature 1..361: note = Synthetic: M6I-07-B05, VH
  source 1..361: mol_type = other DNA

SEQ ID NO: 146 - protein, 106 aa, synthetic construct
  REGION 1..106: note = Synthetic: M6I-07-B05, VL
DIQMTQSPSSLSASVGDRVTITCRASQDISIYLNWYQQKPGKAPKLLIYTASNLQSGVPSRFSGSGSGTDFTLTISSLQPEDFATYYCQQSYSTLTFGGGTKVEIK

SEQ ID NO: 147 - protein, 6 aa, synthetic construct
  REGION 1..6: note = Synthetic: CDRL1-AA
QDISIY

SEQ ID NO: 148 - skipped

SEQ ID NO: 149 - protein, 8 aa, synthetic construct
  REGION 1..8: note = Synthetic: CDR:3-AA
QQSYSTLT

SEQ ID NO: 150 - DNA, 319 bp, synthetic construct
  misc_feature 1..319: note = Synthetic: M6I-07-B05, VL
  source 1..319: mol_type = other DNA
gatattcagatgacccagagcccatcctccctgtcagcttctgtaggagaccgcgttaccatcacctgtcgtgcctcccaggatattagcatctacctgaactggtatcagcagaagcctggtaaggcaccgaagctgctgatctataccgcctccaacctgcaatctggcgtcccgtcacgctttagtggctctggttctgggaccgacttcactctcaccattagcagtctgcaaccggaggacttcgcgacatactattgccagcagtcctatagcactctgacctttggaggcggtaccaaggtggagatcaaag

SEQ ID NO: 151 - protein, 120 aa, synthetic construct; identical to SEQ ID NO: 131
  REGION 1..120: note = Synthetic: M6I-07-C06, VH

SEQ ID NO: 152 - protein, 8 aa, synthetic construct
  REGION 1..8: note = Synthetic: CDRH1-AA
GFTFSNYA

SEQ ID NO: 153 - protein, 7 aa, synthetic construct
  REGION 1..7: note = Synthetic: CDRH2-AA
ISSGGGG

SEQ ID NO: 154 - protein, 14 aa, synthetic construct
  REGION 1..14: note = Synthetic: CDRH3-AA
YCASPPRDYEGMDV

SEQ ID NO: 155 - DNA, 361 bp, synthetic construct; identical to SEQ ID NO: 135
  misc_feature 1..361: note = Synthetic: M6I-07-C06, VH
  source 1..361: mol_type = other DNA

SEQ ID NO: 156 - protein, 107 aa, synthetic construct
  REGION 1..107: note = Synthetic: M6I-07-C06, VL
DIQMTQSPSSLSASVGDRVTITCRASQQISTYLNWYQQKPGKAPKLLIYATSRLEDGVPSRFSGSGSGTDFTLTISSLQPEDFATYYCQQTYITPRTFGGGTKVEIK

SEQ ID NO: 157 - protein, 6 aa, synthetic construct
  REGION 1..6: note = Synthetic: CDRL1-AA
QQISTY

SEQ ID NO: 158 - skipped

SEQ ID NO: 159 - protein, 9 aa, synthetic construct
  REGION 1..9: note = Synthetic: CDR:3-AA
QQTYITPRT

SEQ ID NO: 160 - DNA, 322 bp, synthetic construct
  misc_feature 1..322: note = Synthetic: M6I-07-C06, VL
  source 1..322: mol_type = other DNA
gatattcagatgacccagagcccatcctccctgtcagcttctgtaggagaccgcgttaccatcacctgtcgtgccagccaacagatttccacttacctgaactggtatcagcagaagcctggtaaggcaccgaagctgctgatctatgccaccagccgtctggaagatggcgtcccgtcacgctttagtggctctggttctgggaccgacttcactctcaccattagcagtctgcaaccggaggacttcgcgacatactattgccagcagacatatatcaccccgcgcacctttggaggcggtaccaaggtggagatcaaag

SEQ ID NO: 161 - protein, 119 aa, synthetic construct
  REGION 1..119: note = Synthetic: M6I-07-D05, VH
EVQLLESGGGLVQPGGSLRLSCAASGFPFSHYAMSWVRQAPGKGLEWVSGISGSGHTTDYADSVKGRFTISRDNSKNTLYLQMNSLRAEDTAVYYCARARFPPYYLDIWGQGTLVTVSS

SEQ ID NO: 162 - protein, 8 aa, synthetic construct
  REGION 1..8: note = Synthetic: CDRH1-AA
GFPFSHYA

SEQ ID NO: 163 - protein, 8 aa, synthetic construct
  REGION 1..8: note = Synthetic: CDRH2-AA
ISGSGHTT

SEQ ID NO: 164 - protein, 12 aa, synthetic construct
  REGION 1..12: note = Synthetic: CDRH3-AA
ARARFPPYYLDI

SEQ ID NO: 165 - DNA, 358 bp, synthetic construct
  misc_feature 1..358: note = Synthetic: M6I-07-D05, VH
  source 1..358: mol_type = other DNA
gaggtgcagctcttagaaagcggaggtggcttggttcagcctggcggtagtctaagactgtcctgtgcagcttctggcttcccgtttagccactatgccatgtcttgggtccgtcaagcaccaggtaagggtctggaatgggtgtctggcatctccggcagcggtcacaccactgattatgccgatagcgtgaaaggacgcttcaccatcagcagggacaattccaagaacacgctgtacctgcaaatgaacagcctgcgagccgaggatacggccgtgtattactgtgctcgcgctcggtttcccccttactacttagacatctggggccagggaaccctggtcaccgtctcctcag

SEQ ID NO: 166 - protein, 106 aa, synthetic construct
  REGION 1..106: note = Synthetic: M6I-07-D05, VL
DIQMTQSPSSLSASVGDRVTITCRASHSIGAYLNWYQQKPGKAPKLLIYAASSLQSGVPSRFSGSGSGTDFTLTISSLQPEDFATYYCQQSYSTPKFGGGTKVEIK

SEQ ID NO: 167 - protein, 6 aa, synthetic construct
  REGION 1..6: note = Synthetic: CDRL1-AA
HSIGAY

SEQ ID NO: 168 - skipped

SEQ ID NO: 169 - protein, 8 aa, synthetic construct
  REGION 1..8: note = Synthetic: CDR:3-AA
QQSYSTPK

SEQ ID NO: 170 - DNA, 319 bp, synthetic construct
  misc_feature 1..319: note = Synthetic: M6I-07-D05, VL
  source 1..319: mol_type = other DNA
gatattcagatgacccagagcccatcctccctgtcagcttctgtaggagaccgcgttaccatcacctgtcgtgcctcccacagcattggcgcatacctgaactggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagtcctatagcactccgaaatttggaggcggtaccaaggtggagatcaaag

SEQ ID NO: 171 - protein, 120 aa, synthetic construct; identical to SEQ ID NO: 131
  REGION 1..120: note = Synthetic: M6I-07-E01, VH

SEQ ID NO: 172 - protein, 8 aa, synthetic construct
  REGION 1..8: note = Synthetic: CDRH1-AA
GFTFSNYA

SEQ ID NO: 173 - protein, 7 aa, synthetic construct
  REGION 1..7: note = Synthetic: CDRH2-AA
ISSGGGG

SEQ ID NO: 174 - protein, 14 aa, synthetic construct
  REGION 1..14: note = Synthetic: CDRH3-AA
YCASPPRDYEGMDV

SEQ ID NO: 175 - DNA, 361 bp, synthetic construct; identical to SEQ ID NO: 135
  misc_feature 1..361: note = Synthetic: M6I-07-E01, VH
  source 1..361: mol_type = other DNA

SEQ ID NO: 176 - protein, 107 aa, synthetic construct; identical to SEQ ID NO: 156
  REGION 1..107: note = Synthetic: M6I-07-E01, VL

SEQ ID NO: 177 - protein, 6 aa, synthetic construct
  REGION 1..6: note = Synthetic: CDRL1-AA
QQISTY

SEQ ID NO: 178 - skipped

SEQ ID NO: 179 - protein, 9 aa, synthetic construct
  REGION 1..9: note = Synthetic: CDR:3-AA
QQTYITPRT

SEQ ID NO: 180 - DNA, 322 bp, synthetic construct; identical to SEQ ID NO: 160
  misc_feature 1..322: note = Synthetic: M6I-07-E01, VL
  source 1..322: mol_type = other DNA

SEQ ID NO: 181 - protein, 120 aa, synthetic construct; identical to SEQ ID NO: 131
  REGION 1..120: note = Synthetic: M6I-07-F05, VH

SEQ ID NO: 182 - protein, 8 aa, synthetic construct
  REGION 1..8: note = Synthetic: CDRH1-AA
GFTFSNYA

SEQ ID NO: 183 - protein, 7 aa, synthetic construct
  REGION 1..7: note = Synthetic: CDRH2-AA
ISSGGGG

SEQ ID NO: 184 - protein, 14 aa, synthetic construct
  REGION 1..14: note = Synthetic: CDRH3-AA
YCASPPRDYEGMDV

SEQ ID NO: 185 - DNA, 361 bp, synthetic construct; identical to SEQ ID NO: 135
  misc_feature 1..361: note = Synthetic: M6I-07-F05, VH
  source 1..361: mol_type = other DNA

SEQ ID NO: 186 - protein, 107 aa, synthetic construct; identical to SEQ ID NO: 156
  REGION 1..107: note = Synthetic: M6I-07-F05, VL

SEQ ID NO: 187 - protein, 6 aa, synthetic construct
  REGION 1..6: note = Synthetic: CDRL1-AA
QQISTY

SEQ ID NO: 188 - skipped

SEQ ID NO: 189 - protein, 9 aa, synthetic construct
  REGION 1..9: note = Synthetic: CDR:3-AA
QQTYITPRT

SEQ ID NO: 190 - DNA, 322 bp, synthetic construct; identical to SEQ ID NO: 160
  misc_feature 1..322: note = Synthetic: M6I-07-F05, VL
  source 1..322: mol_type = other DNA

SEQ ID NO: 191 - protein, 119 aa, synthetic construct
  REGION 1..119: note = Synthetic: M6I-07-F06, VH
EVQLLESGGGLVQPGGSLRLSCAASGFTFSNYAMGWVRQAPGKGLEWVSVISGRGGHTYYADSVKGRFTISRDNSKNTLYLQMNSLRAEDTAVYYCASPPIRDAGMDVWGQGTLVTVSS

SEQ ID NO: 192 - protein, 8 aa, synthetic construct
  REGION 1..8: note = Synthetic: CDRH1-AA
GFTFSNYA

SEQ ID NO: 193 - protein, 8 aa, synthetic construct
  REGION 1..8: note = Synthetic: CDRH2-AA
ISGRGGHT

SEQ ID NO: 194 - protein, 12 aa, synthetic construct
  REGION 1..12: note = Synthetic: CDRH3-AA
ASPPIRDAGMDV

SEQ ID NO: 195 - DNA, 357 bp, synthetic construct
  misc_feature 1..357: note = Synthetic: M6I-07-F06, VH
  source 1..357: mol_type = other DNA
gaggtgcagctcttagaaagcggaggtggcttggttcagcctggcggtagtctaagactgtcctgtgcagcttctggcttcacctttagcaactatgcgatgggttgggtccgtcaagcaccaggtaagggtctggaatgggtgtctgtcatctccggtcgcggcggccacacctattatgccgatagcgtgaaaggacgcttcaccatcagcagggacaattccaagaacacgctgtacctgcaaatgaacagcctgcgagccgaggatacggccgtgtattactgtgcttcacctcctattcgagatgctggaatggatgtttggggccagggaaccctggtcaccgtctcctca

SEQ ID NO: 196 - protein, 107 aa, synthetic construct
  REGION 1..107: note = Synthetic: M6I-07-F06, VL
DIQMTQSPSSLSASVGDRVTITCRASQSVNRLLNWYQQKPGKAPKLLIYAASSLQSGVPSRFSGSGSGTDFTLTISSLQPEDFATYYCQQSYSIPHTFGGGTKVEIK

SEQ ID NO: 197 - protein, 6 aa, synthetic construct
  REGION 1..6: note = Synthetic: CDRL1-AA
QSVNRL

SEQ ID NO: 198 - skipped

SEQ ID NO: 199 - protein, 9 aa, synthetic construct
  REGION 1..9: note = Synthetic: CDR:3-AA
QQSYSIPHT

SEQ ID NO: 200 - DNA, 322 bp, synthetic construct
  misc_feature 1..322: note = Synthetic: M6I-07-F06, VL
  source 1..322: mol_type = other DNA
gatattcagatgacccagagcccatcctccctgtcagcttctgtaggagaccgcgttaccatcacctgtcgtgccagccagtccgtgaatcgcctgctgaactggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagtcctatagcatcccgcacacctttggaggcggtaccaaggtggagatcaaag

SEQ ID NO: 201 - protein, 116 aa, synthetic construct
  REGION 1..116: note = Synthetic: M6I-07-G03, VH
EVQLLESGGGLVQPGGSLRLSCAASGFTFSNYVMGWVRQAPGKGLEWVSTITGPGGSTYYADSVKGRFTISRDNSKNTLYLQMNSLRAEDTAVYYCARDLYGFDVWGQGTLVTVSS

SEQ ID NO: 202 - protein, 8 aa, synthetic construct
  REGION 1..8: note = Synthetic: CDRH1-AA
GFTFSNYV

SEQ ID NO: 203 - protein, 8 aa, synthetic construct
  REGION 1..8: note = Synthetic: CDRH2-AA
ITGPGGST

SEQ ID NO: 204 - protein, 9 aa, synthetic construct
  REGION 1..9: note = Synthetic: CDRH3-AA
ARDLYGFDV

SEQ ID NO: 205 - DNA, 349 bp, synthetic construct
  misc_feature 1..349: note = Synthetic: M6I-07-G03, VH
  source 1..349: mol_type = other DNA
gaggtgcagctcttagaaagcggaggtggcttggttcagcctggcggtagtctaagactgtcctgtgcagcttctggttttaccttcagcaactatgtcatgggctgggtccgtcaagcaccaggtaagggtctggaatgggtgtctactatcaccggcccaggtgggagcacctactatgccgatagcgtgaaaggacgcttcaccatcagcagggacaattccaagaacacgctgtacctgcaaatgaacagcctgcgagccgaggatacggccgtgtattactgtgcacgcgacctgtacggcttcgacgtgtggggccagggaaccctggtcaccgtctcctcag

SEQ ID NO: 206 - protein, 107 aa, synthetic construct
  REGION 1..107: note = Synthetic: M6I-07-G03, VL
DIQMTQSPSSLSASVGDRVTITCRASQSIGTHVNWYQQKPGKAPKLLIYAASSLQSGVPSRFSGSGSGTDFTLTISSLQPEDFATYYCQQTYITPRTFGGGTKVEIK

SEQ ID NO: 207 - protein, 6 aa, synthetic construct
  REGION 1..6: note = Synthetic: CDRL1-AA
QSIGTH

SEQ ID NO: 208 - skipped

SEQ ID NO: 209 - protein, 9 aa, synthetic construct
  REGION 1..9: note = Synthetic: CDR:3-AA
QQTYITPRT

SEQ ID NO: 210 - DNA, 322 bp, synthetic construct
  misc_feature 1..322: note = Synthetic: M6I-07-G03, VL
  source 1..322: mol_type = other DNA
gatattcagatgacccagagcccatcctccctgtcagcttctgtaggagaccgcgttaccatcacctgtcgtgcctcccagagcattggcactcacgtgaactggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagacatatatcaccccgcgcacctttggaggcggtaccaaggtggagatcaaag

SEQ ID NO: 211 - protein, 120 aa, synthetic construct
  REGION 1..120: note = Synthetic: M6I-07-H01, VH
EVQLLESGGGLVQPGGSLRLSCAASGFNFANYAMNWVRQAPGKGLEWVSAVSGSGDYTHYADSVKGRFTISRDNSKNTLYLQMNSLRAEDTAVYYCARDFWWGLEGMDYWGQGTLVTVSS

SEQ ID NO: 212 - protein, 8 aa, synthetic construct
  REGION 1..8: note = Synthetic: CDRH1-AA
GFNFANYA

SEQ ID NO: 213 - protein, 8 aa, synthetic construct
  REGION 1..8: note = Synthetic: CDRH2-AA
VSGSGDYT

SEQ ID NO: 214 - protein, 13 aa, synthetic construct
  REGION 1..13: note = Synthetic: CDRH3-AA
ARDFWWGLEGMDY

SEQ ID NO: 215 - DNA, 361 bp, synthetic construct
  misc_feature 1..361: note = Synthetic: M6I-07-H01, VH
  source 1..361: mol_type = other DNA
gaggtgcagctcttagaaagcggaggtggcttggttcagcctggcggtagtctaagactgtcatgtgcagcttctggcttcaactttgctaactacgcgatgaattgggtccgtcaagcaccaggtaagggtctggaatgggtgtctgctgtcagcggcagcggtgattatacccactatgccgatagcgtgaaaggacgcttcaccatcagcagggacaattccaagaacacgctgtacctgcaaatgaacagcctgcgagccgaggatacggccgtgtattactgtgctagagatttctggtgggggctggagggaatggactattggggccagggaaccctggtcaccgtctcctcag

SEQ ID NO: 216 - protein, 107 aa, synthetic construct
  REGION 1..107: note = Synthetic: M6I-07-H01, VL
DIQMTQSPSSLSASVGDRVTITCRASQTIKNYVNWYQQKPGKAPKLLIYAASNFQSGVPSRFSGSGSGTDFTLTISSLQPEDFATYYCQQSYSIPYSFGGGTKVEIK

SEQ ID NO: 217 - protein, 6 aa, synthetic construct
  REGION 1..6: note = Synthetic: CDRL1-AA
QTIKNY

SEQ ID NO: 218 - skipped

SEQ ID NO: 219 - protein, 9 aa, synthetic construct
  REGION 1..9: note = Synthetic: CDR:3-AA
QQSYSIPYS

SEQ ID NO: 220 - DNA, 322 bp, synthetic construct
  misc_feature 1..322: note = Synthetic: M6I-07-H01, VL
  source 1..322: mol_type = other DNA
gatattcagatgacccagagcccatcctccctgtcagcttctgtaggagaccgcgttaccatcacctgtcgtgccagccagaccattaagaactacgtgaattggtatcagcagaagcctggtaaggcaccgaagctgctgatctatgcagcctccaactttcagagcggcgtcccgtcacgctttagtggctctggttctgggaccgacttcactctcaccattagcagtctgcaaccggaggacttcgcgacatactattgccagcagtcctacagcatcccgtattcttttggaggcggtaccaaggtggagatcaaag

SEQ ID NO: 221 - protein, 120 aa, synthetic construct; identical to SEQ ID NO: 131
  REGION 1..120: note = Synthetic: M6I-07-H04, VH

SEQ ID NO: 222 - protein, 8 aa, synthetic construct
  REGION 1..8: note = Synthetic: CDRH1-AA
GFTFSNYA

SEQ ID NO: 223 - protein, 7 aa, synthetic construct
  REGION 1..7: note = Synthetic: CDRH2-AA
ISSGGGG

SEQ ID NO: 224 - protein, 14 aa, synthetic construct
  REGION 1..14: note = Synthetic: CDRH3-AA
YCASPPRDYEGMDV

SEQ ID NO: 225 - DNA, 361 bp, synthetic construct; identical to SEQ ID NO: 135
  misc_feature 1..361: note = Synthetic: M6I-07-H04, VH
  source 1..361: mol_type = other DNA

SEQ ID NO: 226 - protein, 107 aa, synthetic construct; identical to SEQ ID NO: 156
  REGION 1..107: note = Synthetic: M6I-07-H04, VL

SEQ ID NO: 227 - protein, 6 aa, synthetic construct
  REGION 1..6: note = Synthetic: CDRL1-AA
QQISTY

SEQ ID NO: 228 - skipped

SEQ ID NO: 229 - protein, 9 aa, synthetic construct
  REGION 1..9: note = Synthetic: CDR:3-AA
QQTYITPRT

SEQ ID NO: 230 - DNA, 322 bp, synthetic construct; identical to SEQ ID NO: 160
  misc_feature 1..322: note = Synthetic: M6I-07-H04, VL
  source 1..322: mol_type = other DNA

SEQ ID NO: 231 - protein, 120 aa, synthetic construct
  REGION 1..120: note = Synthetic: M6I-07-H05, VH
EVQLLESGGGLVQPGGSLRLSCAASGFSLSDYDMTWVRQAPGKGLEWVSTIRASDESTYYADSVKGRFTISRDNSKNTLYLQMNSLRAEDTAVYYCASGSGALYDPFDVWGQGTLVTVSS

SEQ ID NO: 232 - protein, 8 aa, synthetic construct
  REGION 1..8: note = Synthetic: CDRH1-AA
GFSLSDYD

SEQ ID NO: 233 - protein, 8 aa, synthetic construct
  REGION 1..8: note = Synthetic: CDRH2-AA
IRASDEST

SEQ ID NO: 234 - protein, 13 aa, synthetic construct
  REGION 1..13: note = Synthetic: CDRH3-AA
ASGSGALYDPFDV

SEQ ID NO: 235 - DNA, 361 bp, synthetic construct
  misc_feature 1..361: note = Synthetic: M6I-07-H05, VH
  source 1..361: mol_type = other DNA
gaggtgcagctcttagaaagcggaggtggcttggttcagcctggcggtagtctaagactgtcctgtgcagcttctggcttttccctgagcgattatgacatgacctgggtccgtcaagcaccaggtaagggtctggaatgggtgtctactatccgcgcaagtgacgagtccacctactatgccgatagcgtgaaaggacgcttcaccatcagcagggacaattccaagaacacgctgtacctgcaaatgaacagcctgcgagccgaggatacggccgtgtattactgtgccagcggttccggagctctgtacgatcccttcgatgtgtggggccagggaaccctggtcaccgtctcctcag

SEQ ID NO: 236 - protein, 107 aa, synthetic construct
  REGION 1..107: note = Synthetic: M6I-07-H05, VL
DIQMTQSPSSLSASVGDRVTITCRASQTIKNYVNWYQQKPGKAPKLLIYAASNFQSGVPSRFSGSGSGTDFTLTISSLQPEDFATYYCQQSYSTPPTFGGGTKVEIK

SEQ ID NO: 237 - protein, 6 aa, synthetic construct
  REGION 1..6: note = Synthetic: CDRL1-AA
QTIKNY

SEQ ID NO: 238 - skipped

SEQ ID NO: 239 - protein, 9 aa, synthetic construct
  REGION 1..9: note = Synthetic: CDR:3-AA
QQSYSTPPT

SEQ ID NO: 240 - DNA, 322 bp, synthetic construct
  misc_feature 1..322: note = Synthetic: M6I-07-H05, VL
  source 1..322: mol_type = other DNA
gatattcagatgacccagagcccatcctccctgtcagcttctgtaggagaccgcgttaccatcacctgtcgtgccagccagaccattaagaactacgtgaattggtatcagcagaagcctggtaaggcaccgaagctgctgatctatgcagcctccaactttcagagcggcgtcccgtcacgctttagtggctctggttctgggaccgacttcactctcaccattagcagtctgcaaccggaggacttcgcgacatactattgccagcagtcctatagcaccccaccgacttttggaggcggtaccaaagtggagatcaaag

SEQ ID NO: 241 - protein, 120 aa, synthetic construct; identical to SEQ ID NO: 131
  REGION 1..120: note = Synthetic: M6I-07-H06, VH

SEQ ID NO: 242 - protein, 8 aa, synthetic construct
  REGION 1..8: note = Synthetic: CDRH1-AA
GFTFSNYA

SEQ ID NO: 243 - protein, 7 aa, synthetic construct
  REGION 1..7: note = Synthetic: CDRH2-AA
ISSGGGG

SEQ ID NO: 244 - protein, 14 aa, synthetic construct
  REGION 1..14: note = Synthetic: CDRH3-AA
YCASPPRDYEGMDV

SEQ ID NO: 245 - DNA, 361 bp, synthetic construct; identical to SEQ ID NO: 135
  misc_feature 1..361: note = Synthetic: M6I-07-H06, VH
  source 1..361: mol_type = other DNA

SEQ ID NO: 246 - protein, 107 aa, synthetic construct
  REGION 1..107: note = Synthetic: M6I-07-H06, VL
DIQMTQSPSSLSASVGDRVTITCRASQQISTYLNWYQQKPGKAPKLLIYATSNLHGGVPSRFSGSGSGTDFTLTISSLQPEDFATYYCQQTYITPRTFGGGTKVEIK

SEQ ID NO: 247 - protein, 6 aa, synthetic construct
  REGION 1..6: note = Synthetic: CDRL1-AA
QQISTY

SEQ ID NO: 248 - skipped

SEQ ID NO: 249 - protein, 9 aa, synthetic construct
  REGION 1..9: note = Synthetic: CDR:3-AA
QQTYITPRT

SEQ ID NO: 250 - DNA, 322 bp, synthetic construct
  misc_feature 1..322: note = Synthetic: M6I-07-H06, VL
  source 1..322: mol_type = other DNA
gatattcagatgacccagagcccatcctccctgtcagcttctgtaggagaccgcgttaccatcacctgtcgtgccagccaacagatttccacttacctgaactggtatcagcagaagcctggtaaggcaccgaagctgctgatctatgccaccagcaacctgcacggtggcgtcccgtcacgctttagtggctctggttctgggaccgacttcactctcaccattagcagtctgcaaccggaggacttcgcgacatactattgccagcagacatatatcaccccgcgcacctttggaggcggtaccaaggtggagatcaaag

SEQ ID NO: 251 - protein, 114 aa, synthetic construct
  REGION 1..114: note = Synthetic: M6I-08-A05, VH
QVQLQESGPGLVKPSQTLSLTCTVSGYSITSDYAWNWIRQPPGKGLEWIGYISYSGSTIYNPSLKSRISISVDTSKNQFSLKLNSVTAADTAVYYCATLGLDYWGQGTLVTVSS

SEQ ID NO: 252 - protein, 9 aa, synthetic construct
  REGION 1..9: note = Synthetic: CDRH1-AA
GYSITSDYA

SEQ ID NO: 253 - protein, 7 aa, synthetic construct
  REGION 1..7: note = Synthetic: CDRH2-AA
ISYSGST

SEQ ID NO: 254 - protein, 7 aa, synthetic construct
  REGION 1..7: note = Synthetic: CDRH3-AA
ATLGLDY

SEQ ID NO: 255 - DNA, 343 bp, synthetic construct
  misc_feature 1..343: note = Synthetic: M6I-08-A05, VH
  source 1..343: mol_type = other DNA
caggttcaacttcaggaaagtggtcctgggcttgtcaagccgtctcagacgctctcgctaacctgtactgtgtcagggtactccattacaagtgactatgcctggaattggattcggcagccacccggtaaaggactggagtggattgggtatatctcctatagcggcagcactatatacaatcccagcctgaagtccaggatctcaatcagcgtcgacacctctaaaaaccagttctcactgaagctcaactccgtaaccgctgccgatacagccgtgtactactgcgcaactttaggcctggactattggggacaaggcaccttggttacagtgtcctcag

SEQ ID NO: 256 - protein, 112 aa, synthetic construct
  REGION 1..112: note = Synthetic: M6I-08-A05, VL
DIVMTQSPDSLAVSLGERATINCKSSQSLLYSSNQKNYLAWYQQKPGQPPKLLIYWASTRGSGVPDRFSGSGSGTDFTLTISSLQAEDVAVYYCQQYHSYRTFGQGTKVEIK

SEQ ID NO: 257 - protein, 12 aa, synthetic construct
  REGION 1..12: note = Synthetic: CDRL1-AA
QSLLYSSNQKNY

SEQ ID NO: 258 - skipped

SEQ ID NO: 259 - protein, 8 aa, synthetic construct
  REGION 1..8: note = Synthetic: CDR:3-AA
QQYHSYRT

SEQ ID NO: 260 - DNA, 337 bp, synthetic construct
  misc_feature 1..337: note = Synthetic: M6I-08-A05, VL
  source 1..337: mol_type = other DNA
gatattgtgatgactcaaagccctgactctctagccgtgtccctcggtgagcgggcaacgataaactgcaagtcctctcagtccctgctctatagcagcaaccagaagaattacctggcttggtatcaacagaagcccggacagccgcccaaattgttaatctattgggcttcgacacgcggttcaggcgtaccagacagatttagcgggagtgggtctggcacagatttcacccttactatctccagtctgcaggccgaggacgtggccgtttactactgccagcaataccactcatataggaccttcggccagggaaccaaagtcgaaattaaag

SEQ ID NO: 261 - protein, 117 aa, synthetic construct
  REGION 1..117: note = Synthetic: M6I-08-B05, VH
EVKLVESGGGLVKPGGSLKLSCAASGFTFSTYAMSWVRQTPEKRLEWVATISDGGRYTYYPDSVKGRFTISRDNAKNNLYLQMSSLKSEDTAMYYCARRDGFFFDSWGQGTTLTVSS

SEQ ID NO: 262 - protein, 8 aa, synthetic construct
  REGION 1..8: note = Synthetic: CDRH1-AA
GFTFSTYA

SEQ ID NO: 263 - protein, 8 aa, synthetic construct
  REGION 1..8: note = Synthetic: CDRH2-AA
ISDGGRYT

SEQ ID NO: 264 - protein, 10 aa, synthetic construct
  REGION 1..10: note = Synthetic: CDRH3-AA
ARRDGFFFDS

SEQ ID NO: 265 - DNA, 352 bp, synthetic construct
  misc_feature 1..352: note = Synthetic: M6I-08-B05, VH
  source 1..352: mol_type = other DNA
gaagtgaagctggtggagtctgggggaggcttagtgaagcctggagggtccctgaaactctcctgtgcagcctctggattcactttcagtacctatgccatgtcttgggttcgccagactccggagaagaggctggagtgggtcgcaaccattagtgatggtggtcgttacacctactatccagacagtgtgaaggggcgattcaccatctccagagacaatgccaagaacaacctgtacctgcaaatgagcagtctaaagtctgaggacacagccatgtattactgtgcaagacgggacggtttcttctttgactcctggggccaaggcaccactctcacagtctcctcgg

SEQ ID NO: 266 - protein, 106 aa, synthetic construct
  REGION 1..106: note = Synthetic: M6I-08-B05, VL
DTTVTQSQKFMSTSVGDRVSITCKASQNVGTAVAWYQQKPGQSPKLLIYSASNRYTGVPDRFTGSGSGTDFTLTISSVQAEDLAVYYCKQSYNLYTFGGGTKLEMK

SEQ ID NO: 267 - protein, 6 aa, synthetic construct
  REGION 1..6: note = Synthetic: CDRL1-AA
QNVGTA

SEQ ID NO: 268 - skipped

SEQ ID NO: 269 - protein, 8 aa, synthetic construct
  REGION 1..8: note = Synthetic: CDR:3-AA
KQSYNLYT

SEQ ID NO: 270 - DNA, 319 bp, synthetic construct
  misc_feature 1..319: note = Synthetic: M6I-08-B05, VL
  source 1..319: mol_type = other DNA
gacacaactgtgacccagtctcaaaaattcatgtccacatcagtaggagacagggtcagcatcacctgcaaggccagtcagaatgtgggtactgctgtagcctggtatcaacagaaaccaggacaatctcctaaactactgatttactcggcatccaatcggtacactggagtccctgatcgcttcacaggcagtggatctgggacagatttcactctcaccatcagcagtgtgcaggctgaagacctggcagtttattactgcaagcaatcttataatctgtacacgttcggaggggggaccaagctggaaatgaaac

SEQ ID NO: 271 - protein, 112 aa, synthetic construct
  REGION 1..112: note = Synthetic: M6I-09-A08, VH
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCANWDYWGQGTTLTVSS

SEQ ID NO: 272 - protein, 8 aa, synthetic construct
  REGION 1..8: note = Synthetic: CDRH1-AA
GYTFTSYW

SEQ ID NO: 273 - protein, 8 aa, synthetic construct
  REGION 1..8: note = Synthetic: CDRH2-AA
INPSNGRT

SEQ ID NO: 274 - protein, 5 aa, synthetic construct
  REGION 1..5: note = Synthetic: CDRH3-AA
ANWDY

SEQ ID NO: 275 - DNA, 336 bp, synthetic construct
  misc_feature 1..336: note = Synthetic: M6I-09-A08, VH
  source 1..336: mol_type = other DNA
caggtgcaactgcagcagcctggggctgaactggtgaagcctggggcttcagtgaagctgtcctgcaaggcttctggctacaccttcaccagctactggatgcactgggtgaagcagaggcctggacaaggccttgagtggattggagagattaatcctagcaacggtcgtactaactacaatgagaagttcaagagcaaggccacactgactgtagacaaatcctccagcacagcctacatgcagctcagcagcctgacatctgaggactctgcggtctattactgtgcaaactgggactactggggccaaggcaccactctcacagtctcctcg

SEQ ID NO: 276 - protein, 107 aa, synthetic construct
  REGION 1..107: note = Synthetic: M6I-09-A08, VL
DVVMTQSQKFLSTSVGDRVGVTCKASQNVGTNVAWYQQKPGQSPKLLIYWASTRHTGVPDRFTGSGSGTDFTLTISNVQSEDLADYFCQQYSSYPLTFGGGTKLEMK

SEQ ID NO: 277 - protein, 6 aa, synthetic construct
  REGION 1..6: note = Synthetic: CDRL1-AA
QNVGTN

SEQ ID NO: 278 - skipped

SEQ ID NO: 279 - protein, 9 aa, synthetic construct
  REGION 1..9: note = Synthetic: CDR:3-AA
QQYSSYPLT

SEQ ID NO: 280 - DNA, 322 bp, synthetic construct
  misc_feature 1..322: note = Synthetic: M6I-09-A08, VL
  source 1..322: mol_type = other DNA
gatgttgtgatgacccagtctcaaaaattcttgtccacatcagtaggagacagggtcggcgtcacctgcaaggccagtcagaatgtgggtactaatgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctgacgttcggtggaggcaccaagctggaaatgaaac

SEQ ID NO: 281 - protein, 119 aa, synthetic construct
  REGION 1..119: note = Synthetic: M6I-09-B03, VH
QVQLKESGAELVKPGASVKLSCKASGYTFTNSYIYWVKQRPGQGLEWIGGIDPNNDDTNFNEEFKSKATLTVDKSSSTAYMQLSSLTSEDSAVYYCTRLLRYQAWFAYWGQGTLVTVSS

SEQ ID NO: 282 - protein, 8 aa, synthetic construct
  REGION 1..8: note = Synthetic: CDRH1-AA
GYTFTNSY

SEQ ID NO: 283 - protein, 8 aa, synthetic construct
  REGION 1..8: note = Synthetic: CDRH2-AA
IDPNNDDT

SEQ ID NO: 284 - protein, 12 aa, synthetic construct
  REGION 1..12: note = Synthetic: CDRH3-AA
TRLLRYQAWFAY

SEQ ID NO: 285 - DNA, 358 bp, synthetic construct
  misc_feature 1..358: note = Synthetic: M6I-09-B03, VH
  source 1..358: mol_type = other DNA
caggtgcaactgaaggagtctggggctgaactggtgaagcctggggcttcagtgaaattgtcctgcaaggcttctggctacaccttcaccaactcctatatctactgggtgaagcagaggcctggacaaggccttgagtggattggggggattgatcctaacaatgatgatactaacttcaatgaggagttcaagagcaaggccacactgactgtggacaaatcctccagcacagcctacatgcaactcagcagcctgacatctgaggactctgcggtctattactgtacaagattactacgctaccaggcctggtttgcttactggggccaggggactctggtcaccgtctcctcgg

SEQ ID NO: 286 - protein, 107 aa, synthetic construct
  REGION 1..107: note = Synthetic: M6I-09-B03, VL
DVVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 287 - protein, 6 aa, synthetic construct
  REGION 1..6: note = Synthetic: CDRL1-AA
QNVGTN

SEQ ID NO: 288 - skipped

SEQ ID NO: 289 - protein, 9 aa, synthetic construct
  REGION 1..9: note = Synthetic: CDR:3-AA
QQYNSYPYT

SEQ ID NO: 290 - DNA, 322 bp, synthetic construct
  misc_feature 1..322: note = Synthetic: M6I-09-B03, VL
  source 1..322: mol_type = other DNA
gatgttgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaatcaaac

SEQ ID NO: 291 - protein, 120 aa, synthetic construct
  REGION 1..120: note = Synthetic: M6I-09-D05, VH
QVQLQQPGAELVKPGASVKLSCKASGYTFTNYYIYWVKQRPGQGLEWIGEINPSNGGTHFNEKFESKATLTVDKSSSTTYMQLNSLTSEDSAVYYCVYDGYYRDRYFDVWGAGTTVTVSS

SEQ ID NO: 292 - protein, 8 aa, synthetic construct
  REGION 1..8: note = Synthetic: CDRH1-AA
GYTFTNYY

SEQ ID NO: 293 - protein, 8 aa, synthetic construct
  REGION 1..8: note = Synthetic: CDRH2-AA
INPSNGGT

SEQ ID NO: 294 - protein, 13 aa, synthetic construct
  REGION 1..13: note = Synthetic: CDRH3-AA
VYDGYYRDRYFDV

SEQ ID NO: 295 - DNA, 361 bp, synthetic construct
  misc_feature 1..361: note = Synthetic: M6I-09-D05, VH
  source 1..361: mol_type = other DNA
caggtccaactgcagcagcctggggctgaactggtgaagcctggggcttcagtgaaattgtcctgcaaggcttctggctacaccttcaccaactactatatatactgggtgaaacagaggcctggacaaggccttgagtggattggagagattaatcctagcaatggtggtactcacttcaatgagaagttcgagagcaaggccacactgactgtagacaagtcctccagcacaacatacatgcaactcaacagcctgacatctgaggactctgcggtctattactgtgtctatgatggttactatagggataggtatttcgatgtctggggcgcagggaccacggtcaccgtctcctcgg

SEQ ID NO: 296 - protein, 111 aa, synthetic construct; identical to SEQ ID NO: 106
  REGION 1..111: note = Synthetic: M6I-09-D05, VL

SEQ ID NO: 297 - protein, 10 aa, synthetic construct
  REGION 1..10: note = Synthetic: CDRL1-AA
ESVDSYGNSF

SEQ ID NO: 298 - skipped

SEQ ID NO: 299 - protein, 9 aa, synthetic construct
  REGION 1..9: note = Synthetic: CDR:3-AA
QQNNEDPWT

SEQ ID NO: 300 - DNA, 334 bp, synthetic construct; identical to SEQ ID NO: 110
  misc_feature 1..334: note = Synthetic: M6I-09-D05, VL
  source 1..334: mol_type = other DNA

SEQ ID NO: 301 - protein, 117 aa, synthetic construct
  REGION 1..117: note = Synthetic: M6I-09-F08, VH
EVKLVESGGGLVQPGGSRKLSCAASGFTFSTYAMSWVRQTPEKRLEWVATISDGGRYTYYPDSVKGRFTISRDNAKNNLYLQMSSLKSEDTAMYYCARRDGFFFDSWGQGTTLTVSS

SEQ ID NO: 302 - protein, 8 aa, synthetic construct
  REGION 1..8: note = Synthetic: CDRH1-AA
GFTFSTYA

SEQ ID NO: 303 - protein, 8 aa, synthetic construct
  REGION 1..8: note = Synthetic: CDRH2-AA
ISDGGRYT

SEQ ID NO: 304 - protein, 10 aa, synthetic construct
  REGION 1..10: note = Synthetic: CDRH3-AA
ARRDGFFFDS

SEQ ID NO: 305 - DNA, 352 bp, synthetic construct
  misc_feature 1..352: note = Synthetic: M6I-09-F08, VH
  source 1..352: mol_type = other DNA
gaggtgaagctggtggagtctgggggaggcttagtgcagcctggagggtcccggaaactctcctgtgcagcctctggattcactttcagtacctatgccatgtcttgggttcgccagactccggagaagaggctggagtgggtcgcaaccattagtgatggtggtcgttacacctactatccagacagtgtgaaggggcgattcaccatctccagagacaatgccaagaacaacctgtacctgcaaatgagcagtctaaagtctgaggacacagccatgtattactgtgcaagacgggacggtttcttctttgactcctggggccaaggcaccactctcaccgtctcctcgg

SEQ ID NO: 306 - protein, 108 aa, synthetic construct
  REGION 1..108: note = Synthetic: M6I-09-F08, VL
DIVMTQSQKFMSTSVGDRVSVTCKASQNVGTNVAWYQQKPGQSPKALIYSASYRYSGVPDRFTGSGSGTDFTLTISSMEAEDAAIYYCQQWSSNPPITFGGGTKLEMK

SEQ ID NO: 307 - protein, 6 aa, synthetic construct
  REGION 1..6: note = Synthetic: CDRL1-AA
QNVGTN

SEQ ID NO: 308 - skipped

SEQ ID NO: 309 - protein, 10 aa, synthetic construct
  REGION 1..10: note = Synthetic: CDR:3-AA
QQWSSNPPIT

SEQ ID NO: 310 - DNA, 325 bp, synthetic construct
  misc_feature 1..325: note = Synthetic: M6I-09-F08, VL
  source 1..325: mol_type = other DNA
gatattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcacaatcagcagcatggaggctgaagatgctgccatttattactgccagcagtggagtagtaacccacccatcacgttcggaggggggaccaagctggaaatgaaac

SEQ ID NO: 311 - protein, 112 aa, synthetic construct
  REGION 1..112: note = Synthetic: M6I-10-D03, VH
QVQLQQSGPELKKPGETVRISCKASGYTFTDYSIYWVKQAPGQGLKLMGWINTATGDSTYADDFQGRFAFSLETSASTAYLQINNLKNDDTATYFCARTTFWGQGTTLTVSS

SEQ ID NO: 312 - protein, 8 aa, synthetic construct
  REGION 1..8: note = Synthetic: CDRH1-AA
GYTFTDYS

SEQ ID NO: 313 - protein, 8 aa, synthetic construct
  REGION 1..8: note = Synthetic: CDRH2-AA
INTATGDS

SEQ ID NO: 314 - protein, 5 aa, synthetic construct
  REGION 1..5: note = Synthetic: CDRH3-AA
ARTTF

SEQ ID NO: 315 - DNA, 337 bp, synthetic construct
  misc_feature 1..337: note = Synthetic: M6I-10-D03, VH
  source 1..337: mol_type = other DNA
caggtccaactgcagcagtctggacctgaactgaagaagcctggagagacagtcaggatctcctgcaaggcttctggctataccttcacagactattcaatatactgggtgaagcaggctccaggacagggtttaaagttgatgggctggataaacactgcgactggtgattcaacatatgcggatgacttccagggacggtttgccttctctttggaaacctctgccagcactgcctatttgcagatcaacaacctcaaaaatgacgacacggctacatatttctgtgctaggacgaccttctggggccaaggcaccactctcacagtctcctcgg

SEQ ID NO: 316 - protein, 107 aa, synthetic construct
  REGION 1..107: note = Synthetic: M6I-10-D03, VL
DVVMTQSHKFMSTSVGDRVSVTCKASQNVGTNVAWYQQKPGQSPKALIYSASYRYSGVPDRFTGSGSGTDFTLTISNVQSEDLADYFCQQYNSYPLTFGGGTKLEMK

SEQ ID NO: 317 - protein, 6 aa, synthetic construct
  REGION 1..6: note = Synthetic: CDRL1-AA
QNVGTN

SEQ ID NO: 318 - skipped

SEQ ID NO: 319 - protein, 9 aa, synthetic construct
  REGION 1..9: note = Synthetic: CDR:3-AA
QQYNSYPLT

SEQ ID NO: 320 - DNA, 322 bp, synthetic construct
  misc_feature 1..322: note = Synthetic: M6I-10-D03, VL
  source 1..322: mol_type = other DNA
gatgttgtgatgacccagtctcacaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagactatttctgtcagcaatataacagctatcctctgacgttcggtggaggcaccaagctggaaatgaaac

SEQ ID NO: 321 - protein, 119 aa, synthetic construct; identical to SEQ ID NO: 101
  REGION 1..119: note = Synthetic: M6I-10-D10, VH

SEQ ID NO: 322 - protein, 8 aa, synthetic construct
  REGION 1..8: note = Synthetic: CDRH1-AA
GYTFTNSY

SEQ ID NO: 323 - protein, 8 aa, synthetic construct
  REGION 1..8: note = Synthetic: CDRH2-AA
IDPNNDDT

SEQ ID NO: 324 - protein, 12 aa, synthetic construct
  REGION 1..12: note = Synthetic: CDRH3-AA
TRLLRYQAWFAY

SEQ ID NO: 325 - DNA, 358 bp, synthetic construct; identical to SEQ ID NO: 105
  misc_feature 1..358: note = Synthetic: M6I-10-D10, VH
  source 1..358: mol_type = other DNA

SEQ ID NO: 326 - protein, 111 aa, synthetic construct; identical to SEQ ID NO: 106
  REGION 1..111: note = Synthetic: M6I-10-D10, VL

SEQ ID NO: 327 - protein, 10 aa, synthetic construct
  REGION 1..10: note = Synthetic: CDRL1-AA
ESVDSYGNSF

SEQ ID NO: 328 - skipped

SEQ ID NO: 329 - protein, 9 aa, synthetic construct
  REGION 1..9: note = Synthetic: CDR:3-AA
QQNNEDPWT

SEQ ID NO: 330 - DNA, 334 bp, synthetic construct
  misc_feature 1..334: note = Synthetic: M6I-10-D10, VL
  source 1..334: mol_type = other DNA
gacattgtgctgacccaatctccagcttctttggctgtgtctctagggcagagggccaccatatcstgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaataaaac

SEQ ID NO: 331 - protein, 118 aa, synthetic construct
  REGION 1..118: note = Synthetic: M6I-11-D08, VH
EVKLVESGGGLVKPGGSLKLSCAASGFTFSSFGMHWVRQAPEKGLEWVAYISSGGNYTYYSDSVKGRFTVSRDNAKNTLFLQMSSLRSEDTAMYYCARDDRYDDFDYWGQGTTLTVSS

SEQ ID NO: 332 - protein, 8 aa, synthetic construct
  REGION 1..8: note = Synthetic: CDRH1-AA
GFTFSSFG

SEQ ID NO: 333 - protein, 8 aa, synthetic construct
  REGION 1..8: note = Synthetic: CDRH2-AA
ISSGGNYT

SEQ ID NO: 334 - protein, 11 aa, synthetic construct
  REGION 1..11: note = Synthetic: CDRH3-AA
ARDDRYDDFDY

SEQ ID NO: 335 - DNA, 355 bp, synthetic construct
  misc_feature 1..355: note = Synthetic: M6I-11-D08, VH
  source 1..355: mol_type = other DNA
gaggtgaagctggtggagtctgggggaggcttagtgaagcctggagggtccctgaaactctcctgtgcagcctctggattcactttcagtagctttggaatgcactgggttcgtcaggctccagagaaggggctggagtgggtcgcatacattagtagtggtggtaattacacctactattcagacagtgtgaagggtcgattcaccgtatccagagacaatgccaagaacaccctgttcctgcaaatgagcagtctgaggtctgaggacacggccatgtattactgtgcaagagatgataggtacgacgactttgactactggggccaaggcaccactctcaccgtctcctcgg

SEQ ID NO: 336 - protein, 106 aa, synthetic construct
  REGION 1..106: note = Synthetic: M6I-11-D08, VL
DTTVTQSPAIMSASPGEKVTMTCSASSSVSYMYWYQQKPGSSPRLLIYDTSNLASGVPARFSGSGSGTSYSLTISSMEAEDAATYYCQQYSSYPYTFGGGTKLEIK

SEQ ID NO: 337 - protein, 5 aa, synthetic construct
  REGION 1..5: note = Synthetic: CDRL1-AA
SSVSY

SEQ ID NO: 338 - skipped

SEQ ID NO: 339 - protein, 9 aa, synthetic construct
  REGION 1..9: note = Synthetic: CDR:3-AA
QQYSSYPYT

SEQ ID NO: 340 - DNA, 319 bp, synthetic construct
  misc_feature 1..319: note = Synthetic: M6I-11-D08, VL
  source 1..319: mol_type = other DNA
gacacaactgtgacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgctcgcttcagtggcagtgggtctgggacctcttactctctcacaatcagcagcatggaggctgaagatgctgccacttattactgccagcaatatagcagctatccgtacacgttcggaggggggaccaagctggaaatcaaac

SEQ ID NO: 341 - protein, 118 aa, synthetic construct
  REGION 1..118: note = Synthetic: M6I-11-D10, VH
EVQLVESGGGLVQPGGSRKLSCAASGFAFSTFGMHWIRQAPEKGLEWVASISSGGNYTYYSDSVKGRFTVSRDNAKNTLFLQMSSLRSEDTAMYYCARDDRYDDFDYWGQGTTLTVSS

SEQ ID NO: 342 - protein, 8 aa, synthetic construct
  REGION 1..8: note = Synthetic: CDRH1-AA
GFAFSTFG

SEQ ID NO: 343 - protein, 8 aa, synthetic construct
  REGION 1..8: note = Synthetic: CDRH2-AA
ISSGGNYT

SEQ ID NO: 344 - protein, 11 aa, synthetic construct
  REGION 1..11: note = Synthetic: CDRH3-AA
ARDDRYDDFDY

SEQ ID NO: 345 - DNA, 355 bp, synthetic construct
  misc_feature 1..355: note = Synthetic: M6I-11-D10, VH
  source 1..355: mol_type = other DNA
gaggtgcagctagttgagtctgggggaggcttagtgcagcctggagggtcccggaaactctcctgtgcagcctctggattcgctttcagtacctttggaatgcactggattcgtcaggctccagagaaggggctggagtgggtcgcatccattagtagtggtggtaattacacctactattcagacagtgtgaagggtcgattcaccgtatccagagacaatgccaagaacaccctgttcctgcaaatgagcagtctgaggtctgaggacacggccatgtattactgtgcaagagatgataggtacgacgactttgactactggggccaaggcaccactctcaccgtctcctcgg

SEQ ID NO: 346 - protein, 106 aa, synthetic construct
  REGION 1..106: note = Synthetic: M6I-11-D10, VL
DTTVTQSPAIMSASPGEKVTMTCSASSSVSYMYWYQQKPGSSPRLLIYDTSNLASGVPARFSGSGSGTSYSLTISSMEAEDAATYYCQQWSSNPPTFGGGTKLEIK

SEQ ID NO: 347 - protein, 5 aa, synthetic construct
  REGION 1..5: note = Synthetic: CDRL1-AA
SSVSY

SEQ ID NO: 348 - skipped

SEQ ID NO: 349 - protein, 9 aa, synthetic construct
  REGION 1..9: note = Synthetic: CDR:3-AA
QQWSSNPPT

SEQ ID NO: 350 - DNA, 319 bp, synthetic construct
  misc_feature 1..319: note = Synthetic: M6I-11-D10, VL
  source 1..319: mol_type = other DNA
gacacaactgtgacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgctcgcttcagtggcagtgggtctgggacctcttactctctcacaatcagcagcatggaggctgaagatgctgccacttattactgccagcagtggagtagtaacccacccacgttcggaggggggaccaagctggaaatcaaac

SEQ ID NO: 351 - protein, 120 aa, synthetic construct
  REGION 1..120: note = Synthetic: M6I_02-G04, VH
EVKLVESGGGLVQPGGSLRLSCTTSGFTFTDYYMSWVRQPPGKALEWLGFIRNKANGYTTEYSTSVKGRFTISRDNSHSILYLQMNTLRAEDSATYYCARDYYGSLFAYWGQGTLVTVSA

SEQ ID NO: 352 - protein, 8 aa, synthetic construct
  REGION 1..8: note = Synthetic: CDRH1-AA
GFTFTDYY

SEQ ID NO: 353 - protein, 10 aa, synthetic construct
  REGION 1..10: note = Synthetic: CDRH2-AA
IRNKANGYTT

SEQ ID NO: 354 - protein, 11 aa, synthetic construct
  REGION 1..11: note = Synthetic: CDRH3-AA
ARDYYGSLFAY

SEQ ID NO: 355 - DNA, 361 bp, synthetic construct
  misc_feature 1..361: note = Synthetic: M6I_02-G04, VH
  source 1..361: mol_type = other DNA
gaagtgaagctggtggagtctggaggaggcttggtacagcctgggggttctctgagactctcctgtacaacttctgggttcaccttcactgattactacatgagctgggtccgccagcctccaggaaaggcacttgagtggttgggttttattagaaacaaagctaatggttacacaacagagtacagtacatctgtgaagggtcggttcaccatctccagagataattcccacagcatcctctatcttcaaatgaacaccctgagagctgaggacagtgccacttattactgtgcaagagattactacggtagtttgtttgcttactggggccaagggactctggtcactgtctctgcgg

SEQ ID NO: 356 - protein, 106 aa, synthetic construct
  REGION 1..106: note = Synthetic: M6I_02-G04, VL
DNVLTQSPALMSASPGEKVTMTCSASSSVSYMHWYQQKSGTSPKRWIYDTSKLASGVPARFSGSGSGTSYSLTISSMEAEDAATYYCQQWSSNPPTFGGGTKLEIK

SEQ ID NO: 357 - protein, 5 aa, synthetic construct
  REGION 1..5: note = Synthetic: CDRL1-AA
SSVSY

SEQ ID NO: 358 - skipped

SEQ ID NO: 359 - protein, 9 aa, synthetic construct
  REGION 1..9: note = Synthetic: CDR:3-AA
QQWSSNPPT

SEQ ID NO: 360 - DNA, 318 bp, synthetic construct
  misc_feature 1..318: note = Synthetic: M6I_02-G04, VL
  source 1..318: mol_type = other DNA
gacaatgttctcacccagtctccagcactcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccacgttcggtggaggcaccaagctggaaataaaa

SEQ ID NO: 361 - protein, 122 aa, synthetic construct
  REGION 1..122: note = Synthetic: Hum 1D7, VH
EVQLVESGGGLVKPGGSLRLSCVASGFTFSSFAMSWVRQAPGKGLEWVAIISSGGGYIYYADSMTGRFTISRDNAKNSLYLQMSSLRAEDTAVYYCARLGGDYDEFYAKDYWGQGTLVTVSS

SEQ ID NO: 362 - DNA, 366 bp, synthetic construct
  misc_feature 1..366: note = Synthetic: Hum 1D7, VH
  source 1..366: mol_type = other DNA
gaagtgcaactggtggagtcgggtgggggcttggtaaagcctgggggctctctcagactgagctgtgttgcatcaggattcacattttcttccttcgctatgtcttgggttcggcaggcacccggcaaaggactggaatgggtcgccattatcagttccggaggcggctatatctattacgccgacagcatgactgggcggttcaccatatcaagggacaatgctaagaacagtctctacttacagatgtcctccctacgcgcagaggatacggccgtctactactgcgcccgacttgggggtgattacgacgagttttatgccaaagattattggggtcagggaaccctggtgacagtgagcagc

SEQ ID NO: 363 - protein, 122 aa, synthetic construct
  REGION 1..122: note = Synthetic: Hum 1D7, VH
EVQLVESGGGLVKPGGSLRLSCAASGFTFSSFAMSWVRQAPGKGLEWVSIISSGGGYIYYADSMTGRFTISRDNAKNSLYLQMNSLRAEDTAVYYCARLGGDYDEFYAKDYWGQGTLVTVSS

SEQ ID NO: 364 - DNA, 366 bp, synthetic construct
  misc_feature 1..366: note = Synthetic: Hum 1D7, VH
  source 1..366: mol_type = other DNA
gaagtgcaactggtggaatctggtgggggacttgtaaaacctggcggttcactgaggctctcctgtgccgcctcaggcttcaccttttccagtttcgctatgagttgggtgcgtcaggcacccggcaaggggttagagtgggtgtctattatcagcagcggaggcggctatatatactatgctgattccatgacagggagattcacgatctcgcgggacaacgccaagaacagcttgtacctacagatgaatagcctgcgagccgaggacacagcagtctattactgcgcacgcctcggaggtgattacgatgagttttacgccaaagactattggggacaggggaccctggtcactgtttcttcc

SEQ ID NO: 365 - protein, 122 aa, synthetic construct
  REGION 1..122: note = Synthetic: Hum 1D7, VH
EVQLVESGGGLVQPGGSLRLSCAASGFTFSSFAMSWVRQAPGKGLEWVSIISSGGGYIYYADSMTGRFTISRDNSKNTLYLQMNSLRAEDTAVYYCAKLGGDYDEFYAKDYWGQGTMVTVSS

SEQ ID NO: 366 - DNA, 366 bp, synthetic construct
  misc_feature 1..366: note = Synthetic: Hum 1D7, VH
  source 1..366: mol_type = other DNA
gaggtacaacttgtggaatcaggaggggggctagtccagcctggaggaagtctccgattgtcttgtgctgcctccggattcactttttcttcatttgccatgtcctgggtcagacaggctcccggtaagggcttagagtgggtgagcataatcagcagcgggggcggctatatctattatgcagatagcatgacggggcggttcaccatttcccgcgacaacagtaagaataccctgtacctccagatgaactcgctgagggcagaagacacagcagtttactactgcgccaaactgggcggtgattatgacgagttctacgccaaagattactggggtcagggcacaatggtgactgtgtcctct

SEQ ID NO: 367 - protein, 106 aa, synthetic construct
  REGION 1..106: note = Synthetic: Hum 1D7, VL
DIQMTQSPSSLSASVGDRVTITCKASEDIYNRLAWYQQKPGKAPRLLIYGATSLETGVPSRFSGSGSGTDFTLTITSLQPEDVATYYCQQYWSTWTFGQGTKVEIK

SEQ ID NO: 368 - DNA, 318 bp, synthetic construct
  misc_feature 1..318: note = Synthetic: Hum 1D7, VL
  source 1..318: mol_type = other DNA
gacatccagatgactcagtctccttcaagtctgagcgcttcggtgggcgaccgcgtcacaattacatgcaaagccagtgaagacatctacaaccggctcgcctggtatcaacagaagcccggcaaagcaccaagactgctgatatatggagctacgtccttagagacaggtgtaccgtccaggtttagcgggagcgggtccggcactgatttcactttgaccattacctcacttcagcccgaagacgttgccacctactattgtcagcaatactggtctacctggacattcggccagggaaccaaggtggagatcaag

SEQ ID NO: 369 - protein, 106 aa, synthetic construct
  REGION 1..106: note = Synthetic: Hum 1D7, VL
DIQMTQSPSSLSASVGDRVTITCKASEDIYNRLAWYQQKPGKAPKLLIYGATSLETGVPSRFSGSGSGTDFTLTISSLQPEDVATYYCQQYWSTWTFGQGTKVEIK

SEQ ID NO: 370 - DNA, 318 bp, synthetic construct
  misc_feature 1..318: note = Synthetic: Hum 1D7, VL
  source 1..318: mol_type = other DNA
gacattcagatgacacagtctcctagctcgcttagtgcttccgtgggagatcgcgtgaccatcacatgtaaagcctcagaagacatctacaacagactggcctggtatcagcagaagccagggaaagcacccaagctcctgatatacggtgccacatctttagagacaggcgttccgtccaggttcagcgggagcggttctggaactgactttactttgaccatctccagtctgcaacccgaggatgtcgctacctactattgccaacagtattggtcaacttggacgttcggccagggcaccaaagtggaaattaag

SEQ ID NO: 371 - protein, 106 aa, synthetic construct
  REGION 1..106: note = Synthetic: Hum 1D7, VL
DIQMTQSPSSLSASVGDRVTITCKASEDIYNRLAWYQQKPGKAPKLLIYGATSLETGVPSRFSGSGSGTDFTLTISSLQPEDFATYYCQQYWSTWTFGQGTKVEIK

SEQ ID NO: 372 - DNA, 318 bp, synthetic construct
  misc_feature 1..318: note = Synthetic: Hum 1D7, VL
  source 1..318: mol_type = other DNA
gatatacagatgacacagtctccttcttccctgtcggcttcagtgggtgatcgggttactattacctgcaaggccagcgaggacatctataacaggctggcctggtatcagcagaaacccggaaaggctccgaagctgttaatctatggtgcaacatccctcgaaacaggcgtcccatccagattcagtggctcagggagcggaacagactttacccttacgatctccagtttgcagcccgaggactttgccacctactactgtcagcaatactggagcacttggaccttcggccaaggcactaaagtggaaattaaa

SEQ ID NO: 373 - protein, 59 aa, synthetic construct
  REGION 1..59: note = Synthetic: Epitope 1
NFSPLARRVDRVAIYEEFLRMTRNGTQLQNFTLDRSSVLVDGYSPNRNEPLTGNSDLPC

SEQ ID NO: 374 - protein, 33 aa, synthetic construct
  REGION 1..33: note = Synthetic: Epitope 2
NFTLDRSSVLVDGYSPNRNEPLTGNSDLPGSGC

SEQ ID NO: 375 - protein, 15 aa, synthetic construct
  REGION 1..15: note = Synthetic: Linker
GGGGSGGGGSGGGGS

(19) United States
(12) Patent Application Publication
(10) Pub. No.: US 2024/0262929 A1
(43) Pub. Date: Aug. 8, 2024

(54) ANTI-MUC16 ANTIBODIES

(71) Applicant: iBio, Inc., Bryan, TX (US)

(72) Inventors: Matthew P. Greving, San Carlos, CA (US); Alexander Tomoaki Taguchi, San Mateo, CA (US); Gao Liu, Brisbane, CA (US); Hari Hara S. Potula, Freemont, CA (US); Cody Allen Moore, San Diego, CA (US)

(21) Appl. No.: 18/432,682

(22) Filed: Feb. 5, 2024

Related U.S. Application Data
(60) Provisional application No. 63/443,527, filed on Feb. 6, 2023.

(51) Int. Cl. C07K 16/30; A61K 39/00

(52) U.S. Cl. CPC C07K 16/3092; A61K 2039/505; C07K 2317/24; C07K 2317/52; C07K 2317/565; C07K 2317/622

(57) ABSTRACT

Provided herein are anti-MUC16 antibodies or binding fragments thereof which bind the non-shed domain of MUC16. The anti-MUC16 antibodies or binding fragments thereof of the disclosure are useful for the treatment of cancer cells that shed the extracellular domain of MUC16.

### CROSS-REFERENCE TO RELATED APPLICATIONS

This application claims priority to U.S. Provisional Application Ser. No. 63/443,527, filed Feb. 6, 2023, and, the entire contents of which are incorporated herein by reference.

### TECHNICAL FIELD OF THE INVENTION

This document relates to antibodies, portions thereof, and methods for treating cancer, and particularly, to novel anti-MUC16 antibodies that are used to reduce or eliminate cancer cells that have on their surface the tumor-specific form of MUC16 (MUC16-C), and which have shed the N-terminal (MUC16-N) and tandem repeat domains (MUC16-TR).

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Feb. 5, 2024, is named “IBIO1039.xml” and is 465,851 bytes in size. The sequence listing contained in this .XML file is part of the specification and is hereby incorporated by reference herein in its entirety.

### BACKGROUND OF THE INVENTION

Without limiting the scope of the invention, its background is described in connection with cancer cells that shed MUC-16.

One such composition that targets MUC-16 expressing cancer cells is taught in U.S. Pat. No. 10,738,130, issued to Heber, et al., entitled “Bispecific antigen-binding molecules that bind MUC16 and CD3, and compositions thereof”. These inventors are said to teach full-length human IgG antibodies that bind to human and MUC16 and bispecific antibodies (bsAbs) that bind to both MUC16 and CD3 that activate T cells via the CD3 complex in the presence of MUC16-expressing tumors. The bispecific antigen-binding molecules are also said to include a first antigen-binding domain that specifically binds human and monkey CD3, and a second antigen-binding molecule that specifically binds human and monkey MUC16. In certain embodiments, the bispecific antigen-binding molecules are said to be capable of inhibiting the growth of tumors expressing MUC16. The MUC16-CD3 bispecific T cell engager is known as ubamatamab.

Another composition is taught by Spriggs, et al., in International Patent Publication No. WO2020227538A1, entitled “humanized antibodies to MUCIN-16 and methods of use thereof.” These applicants are said to teach compositions, methods, and uses of anti-Mucin-16 (MUC16) agents that specifically bind to an epitope of MUC16. These applicants are said to further teach uses and methods for managing, treating, or preventing disorders, such as cancer and diseases associated with positive MUC16 expression.

Finally, another composition is taught in U.S. Pat. No. 11,319,380 issued to Sabzevari and Shah, entitled, “MUC16 specific chimeric antigen receptors and uses thereof”. These inventors are said to teach chimeric antigen receptors (CARs) for cancer therapy, and more particularly, CARs containing an scFv from an anti-MUC16 monoclonal antibody. Provided are immune effector cells containing such CARs, and methods of treating proliferative disorders.

Despite these advances, a need remains for agents that specifically target the truncated portion of MUC16 that remains on the surface of cancer cells when the N-terminal end of MUC16 is shed.

### SUMMARY OF THE INVENTION

As embodied and broadly described herein, an aspect of the present disclosure relates to an anti-MUC16 antibody or binding fragment thereof, wherein the antibody comprises: a heavy chain variable domain (VH) complementarity determining region (CDR) 1 comprising the amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359. In one aspect, the antibody comprises: a VH having at least 96, 97, 98, 99, or 100% sequence identity with the amino acid sequence of any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and a VL having at least 96, 97, 98, 99, or 100% sequence identity with the amino acid sequence of any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371. In another aspect, the antibody is a monoclonal antibody. In another aspect, the antibody is a full-length humanized antibody, chimeric antibody, a fusion protein, or an antibody fragment. In another aspect, the CDRs are selected from following three VH CDRs: SEQ ID NOS: 2, 3, 4; 12, 13, 14; 22, 23, 24; 32, 33, 34; 42, 43, 44; 52, 53, 54; 62, 63, 64; 72, 73, 74; 82, 83, 84; 92, 93, 94; 102, 103, 104; 112, 113, 114; 122, 123, 124; 132, 133, 134; 142, 143, 144; 152, 153, 154; 162, 163, 164; 172, 173, 174; 182, 183, 184; 192, 193, 194; 202, 203, 204; 212, 213, 214; 222, 223, 224; 232, 233, 234; 242, 243, 244; 252, 253, 254; 262, 263, 264; 272, 273, 274; 282, 283, 284; 292, 293, 294; 302, 303, 304; 312, 313, 314; 322, 323, 324; 332, 333, 334; 342, 343, 344; and 352, 353, 354; and comprises the amino acid sequences of the following three VL CDRs SEQ ID NOS: 7, 8, 9; 17, 18, 19; 27, 28, 29; 37, 38, 39; 47, 48, 49; 57, 58, 59; 67, 68, 69; 77, 78, 79; 87, 88, 89; 97, 98, 99; 107, 108, 109; 117, 118, 119; 127, 128, 129; 137, 138, 139; 147, 148, 149; 157, 158, 159; 167, 168, 169; 177, 178, 179; 187, 188, 189; 197, 198, 199; 207, 208, 209; 217, 218, 219; 227, 228, 229; 237, 238, 239; 247, 248, 249; 257, 258, 259; 267, 268, 269; 277, 278, 279; 287, 288, 289; 297, 298, 299; 307, 308, 309; 317, 318, 319; 327, 328, 329; 337, 338, 339; 347, 348, 349; and 357, 358, 359. In another aspect, the antibody binding fragment is fused to an Fc domain of any one of the following: human IgG1, human IgG2, human IgG3, and human IgG4.

As embodied and broadly described herein, an aspect of the present disclosure relates to a method of treating a disease in a subject in need thereof, comprising administering to the subject a therapeutically effective amount of the antibody described hereinabove. In one aspect, the disease is an autoimmune disease. In another aspect, the disease is an inflammatory disease. In another aspect, the subject is human.

As embodied and broadly described herein, an aspect of the present disclosure relates to an anti-MUC16 antibody that comprises an scFv1 and an scFv2 binding site in tandem on each antibody arm and wherein said scFv1 and scFv2 are linked by a linker, optionally a flexible linker. In one aspect, the antibody has a total of four scFv binding sites in a single scFv-Fc formatted antibody. In another aspect, the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2). In another aspect, the VH1 region and the VH2 region each comprises the amino acid sequence of a heavy chain variable domain (VH) complementarity determining region (CDR) comprising the amino acid sequence of: a heavy chain variable domain (VH) complementarity determining region (CDR) 1 comprising the amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359. In another aspect, the antibody comprises the amino acid sequences: a VH comprising the amino acid sequence having at least 96, 97, 98, 99, or 100% sequence identity with any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and a VL comprising the amino acid sequence having at least 96, 97, 98, 99, or 100% sequence identity with any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.

As embodied and broadly described herein, an aspect of the present disclosure relates to a method of treating a disease in a subject in need thereof, comprising administering to the subject a therapeutically effective amount of the antibody described hereinabove. In one aspect, the disease is a cancer that expresses MUC16. In another aspect, the cancer is selected from ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16. In another aspect, the subject is human.

As embodied and broadly described herein, an aspect of the present disclosure relates to a nucleic acid that expresses an anti-MUC16 antibody or binding fragment thereof, wherein the antibody comprises: a nucleic acid having at least 95% sequence identity to a heavy chain variable domain that encodes a heavy chain variable domain (VH) comprising complementarity determining region (CDR) 1 of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and a nucleic acid having at least 95% sequence identity to a heavy chain variable domain that encodes a heavy chain variable domain (VH) comprising a CDR2 of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and a nucleic acid having at least 95% sequence identity to a heavy chain variable domain that encodes a heavy chain variable domain (VH) comprising a VH CDR3 comprising any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and a nucleic acid having at least 95% sequence identity to a light chain variable domain that encodes a light chain variable domain (VL) comprising a VL CDR1 comprising any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and a nucleic acid having at least 95% sequence identity to a light chain variable domain that encodes a light chain variable domain (VL) comprising a VL CDR2 comprising any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and a nucleic acid having at least 95% sequence identity to a light chain variable domain that encodes a light chain variable domain (VL) comprising a VL CDR3 comprising any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359. In another aspect, the VH is a nucleic acid having at least 96, 97, 98, 99, or 100% sequence identity with SEQ ID NO: 5; 15; 25; 35; 45; 55; 65; 75; 85; 95; 105; 115; 125; 135; 145; 155; 165; 175; 185; 195; 205; 215; 225; 235; 245; 255; 265; 275; 285; 295; 305; 315; 325; 335; 345; or 355, and the VL is a nucleic acid having at least 95, 96, 97, 98, 99, or 100% sequence identity with SEQ ID NO: 10; 20; 30; 40; 50; 60; 70; 80; 90; 100; 110; 120; 130; 140; 150; 160; 170; 180; 190; 200; 210; 220; 230; 240; 250; 260; 270; 280; 290; 300; 310; 320; 330; 340; 350, or a humanized version thereof.

As embodied and broadly described herein, an aspect of the present disclosure relates to a vector that comprises the nucleic acid of any one of the amino acids and nucleic acids described hereinabove.

As embodied and broadly described herein, an aspect of the present disclosure relates to a host cell that comprises the vector(s) described hereinabove.

### BRIEF DESCRIPTION OF THE DRAWINGS

For a more complete understanding of the features and advantages of the present invention, reference is now made to the detailed description of the invention along with the accompanying figures and in which:

FIG. 1A illustrates the methods for steering immunizations to a MUC16 Epitope that avoids tumor resistance.

FIG. 1B are graphs for the cell binding screen and a western blot that show the results from the immunization strategies by cell binding and western blot.

FIG. 2A shows the epitope designed for immunization of engineered epitope 1, which is a 58-mer of MUC16 non-shed domain, including the glycosylation sites. Engineered epitope 1 was expressed in mammalian cells and was used in glycosylated form.

FIG. 2B shows the epitope designed for immunization of engineered epitope 2, which is a 29-mer of MUC16 non-shed domain, showing the glycosylation site. Engineered epitope 2 was produced synthetically and was used in aglycosylated form.

FIG. 3 is a graph that shows an ELISA dose-response curve of clone 1D7 binding to both the engineered epitope 1 and engineered epitope 2 peptides.

FIG. 4 shows the location of the 29-mer and the non-shed 58-mer in relation to the entire length of MUC16, including the shed domain, the non-shed domain and the location of the aglycosylated non-shed 29-mer and 58-mer in relation to the intracellular and extracellular domains of MUC16.

FIG. 5A are graphs that show that the 1D7 anti-MUC16 clone binds a membrane-proximal aglycosylated epitope and does not bind the MUC16 shed domain.

FIG. 5B is a graph that shows that the 1D7 anti-MUC16 clone specifically binds MUC16_{high} vs. MUC16_{low} cells.

FIG. 5C is a western blot that shows the binding of the 1D7 anti-MUC16 clone to OVCAR3 ovarian cancer cells that have a high expression of MUC 16, and SKOV3 ovarian cancer cells that have a low expression of MUC 16, using actin as a control for protein loading.

FIG. 6 is a graph that shows an ELISA dose-response curve of clone 8G4 binding to both the engineered epitope 1 and engineered epitope 2 peptides.

FIG. 7A are graphs that show that the 8G4 anti-MUC16 clone binds a membrane-proximal aglycosylated epitope and does not bind the MUC16 shed domain.

FIG. 7B is a graph that shows that the 8G4 anti-MUC16 clone specifically binds MUC16_{high} vs. MUC16_{low} cells.

FIG. 7C is a western blot that shows the binding of the 8G4 anti-MUC16 clone to OVCAR3 ovarian cancer cells that have a high expression of MUC 16, and SKOV3 ovarian cancer cells that have a low expression of MUC 16, using actin as a control for protein loading.

FIG. 8 is a graph that shows an ELISA dose-response curve of clone 21G6 anti-MUC16 clone identified in a hybridoma ELISA screen using the engineered 58-mer antigen—engineered epitope design 1.

FIG. 9A is a graph that shows 21G6 Anti-MUC16 clone specifically binds OVCAR3 cells that are ovarian cancer cells expressing high levels of MUC16_{high}.

FIG. 9B are graphs that show the binding of 21G6 anti-MUC16 clone to SKOV3 MUC16_{low} cells and MUC16(−) HEK293 cells.

FIG. 10 are graphs that show that the 21G6 anti-MUC16 clone binds a membrane-proximal aglycosylated epitope and does not bind the MUC16 shed domain.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

The term “antibody” as used herein throughout is used in the broadest sense and includes a monoclonal antibody, polyclonal antibody, human antibody, humanized antibody, non-human antibody, chimeric antibody, a monovalent antibody, an antibody fragment, and a tandem scFv-Fc antibody.

Antibody fragments of the disclosure retain MUC-16 antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

The term “meso scale-molecule (MEM)” as used herein throughout includes engineered peptides and polypeptides between about 1 kDa and about 10 kDa. The term “MEM-nanoparticle” as used herein throughout includes MEMs which have been conjugated to a nanoparticle (e.g., ferritin nanoparticle).

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

All publications, patents, and patent applications mentioned in this specification are herein incorporated by reference to the same extent as if each individual publication, patent, or patent application was specifically and individually indicated to be incorporated by reference.

### Antibodies.

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)₂. Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab)₂. Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab)₂ fragments.

As used herein, the “Fv” fragment is the minimum antibody fragment that contains a complete antigen recognition and binding site. This region includes a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequenator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a ß-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.). The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA. IgD. IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies and is not to be construed as requiring the production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for MUC-16 is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab)₂. Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fᵥ framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against MUC16. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007. (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the antibody or binding fragments thereof. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in W0/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that the effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational, or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as 9, 10, 12, or 15 amino acids. The term includes polypeptides, which include modifications, such as deletions, additions, and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a protein, peptide, carbohydrate, small molecule, lipid, etc.) that when present in the proper conformation, provides a binding or reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to the isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, and sedimentation according to density.

As used herein, the term “treatment” refers to any of (i) the prevention or elimination of target cells, e.g., cancer cells that express MUC16 or its variants, as in a traditional vaccine, (ii) the reduction or elimination of symptoms, and (iii) the substantial or complete elimination of the target cell in question. Treatment may be effected prophylactically (prior to the presence or spread of a cancer cell) or therapeutically (following identification of the cancer cell).

The practice of the present invention employs, unless otherwise indicated, conventional methods of chemistry, biochemistry, molecular biology, immunology, and pharmacology, within the skill of the art. Such techniques are explained fully in the literature. Sec, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Provided herein are anti-MUC16 antibodies and binding fragments thereof that specifically bind to MUC16, and in particular, to the MUC16-C domain, which includes the intracellular domain, transmembrane domain, and a short extracellular domain from which the MUC16-TR (tandem repeats) and MUC16-N(N-terminal) domains has been cleaved. Thus, in some embodiments, the anti-MUC16 antibodies and binding fragments thereof specifically bind to the retained extracellular domain of MUC16. In some embodiments, the anti-MUC16 antibodies and binding fragments thereof include an anti-MUC16 antibody binding fragment that specifically binds to MUC16-C with or without post-translational modification, e.g., glycosylation. In some embodiments, the anti-MUC16 antibodies and binding fragments thereof are a full-length anti-MUC16 antibody, an antigen-binding fragment thereof, or a chimera with another antibody or protein. In some embodiments, the antibodies and binding fragments thereof specifically bind to a MUC16-C expressing cell (e.g., a MUC 16-expressing cancer cell).

The anti-MUC 16 antibodies or antigen-binding fragments thereof, can include, e.g., monoclonal antibodies, polyclonal antibodies, recombinantly produced antibodies, monospecific antibodies, multispecific antibodies, bispecific antibodies, chimeric antigen receptors (CAR), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, single domain antibodies, monovalent antibodies, single chain antibodies or single-chain variable fragments (scFv), camelid antibodies, affybodies, disulfide-linked Fv (dsFv), Fc fusion proteins, immunoconjugates, or fragments thereof. Such antibodies and antigen-binding fragments can be made by methods known in the art.

In some embodiments, the anti-MUC16 antibodies or antigen-binding fragments thereof is a full-length antibody (e.g., full-length IgG) or antigen-binding fragment thereof, which specifically binds to MUC 16.

In some embodiments, reference to antibodies and binding fragments thereof that specifically bind to MUC16 refers to antibodies or antigen-binding fragments thereof that bind to MUC16 with an affinity that is at least about 10 times (including for example at least about any of 10, 102, 103, 104, 105, 106, 107, 108, 109, or more times) its binding affinity for a non-target antigen, that is, an antigen that is not MUC16. Binding affinity can be determined by any methods known in the art, such as, e.g., ELISA, fluorescence-activated cell sorting (FACS) analysis, or radioimmunoprecipitation assay (RIA). Kd can be determined by methods known in the art, such as surface plasmon resonance (SPR) assay utilizing (Biacore), or kinetic exclusion assay (KinExA)(Sapidyne instruments).

Although anti-MUC16 antibodies or antigen-binding fragments thereof containing human sequences (e.g., human heavy and light chain variable domain sequences comprising human CDR sequences) are extensively discussed herein, non-human anti-MUC16 antibodies or antigen-binding fragments thereof are also taught. In some embodiments, non-human anti-MUC16 antibodies and binding fragments thereof comprise human CDR sequences from the anti-MUC16 antibodies and binding fragments thereof described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, buffalo), deer, sheep, goat, chicken, cat, dog, ferret, primate (e.g., rhesus monkey), etc. In some embodiments, a non-human anti-MUC16 antibodies or antigen-binding fragments thereof includes an anti-MUC16 antibodies or antigen-binding fragments thereof generated by grafting or veneering one or more CDR sequences as described herein onto a human framework sequence.

As used herein, the term “chimeric antigen receptor (CAR)” refers to an artificially constructed hybrid single-chain protein or single-chain polypeptide containing a single-chain variable fragment (scFv) as a part of the extracellular antigen-binding domain, linked directly or indirectly to a transmembrane domain (e.g., an immune cell co-stimulatory signaling molecule transmembrane domain), which is in turn linked directly or indirectly to an intracellular immune cell (e.g., T cell or NK cell) signaling domain. The intracellular signaling domain (ISD) comprises a primary signaling sequence, or primary immune cell signaling sequence, from an antigen-dependent, TCR-associated T cell activation molecule, e.g., a portion of the intracellular domain of CD3zeta, TOIz, FcRy, FcRP, CD3y, CD35, CD3e, CD5, CD22, CD79a, CD79b, or CD66d). The ISD can further comprise a co-stimulatory signaling sequence, e.g., a portion of the intracellular domain of an antigen-independent, co-stimulatory molecule such as CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, or the like. Characteristics of CARs include their ability to redirect immune cell (e.g., T cell or NK cell) specificity and reactivity toward a selected target in either MHC-restricted (in cases of TCR-mimic antibodies) or non-MHC-restricted (in cases of antibodies against cell surface proteins) manners, exploiting the antigen binding properties of monoclonal antibodies. The non-MHC-restricted antigen recognition gives immune cells (e.g., T cells or NK cells) expressing CARs the ability to recognize antigens independent of antigen processing, thus bypassing a major mechanism of tumor escape.

In some embodiments, the anti-MUC 16 antibodies or antigen-binding fragments are a chimeric co-stimulatory receptor comprising an anti-MUC 16 antigen-binding fragment that specifically binds to MUC16 and a co-stimulatory signaling domain. In some embodiments, the anti-MUC 16 chimeric co-stimulatory receptor is capable of stimulating an immune cell adjacent or in contact with a target cell, the target cell expressing MUC16, and in particular, MUC16-C. In some embodiments, the anti-MUC16 chimeric co-stimulatory receptor lacks a functional primary immune cell signaling sequence. In some embodiments, the anti-MUC16 chimeric co-stimulatory receptor lacks any primary immune cell signaling sequence. In some embodiments, the anti-MUC16 chimeric co-stimulatory receptor comprises a single polypeptide chain comprising the anti-MUC16 antigen-binding fragments, a transmembrane domain, and the co-stimulatory signaling domain. In some embodiments, the anti-MUC16 chimeric co-stimulatory receptor comprises a first polypeptide chain and a second polypeptide chain, wherein the first and second polypeptide chains together form the anti-MUC16 antibody moiety, a transmembrane module, and co-stimulatory signaling module comprising the co-stimulatory signaling domain. In some embodiments, the first and second polypeptide chains are separate polypeptide chains, and the anti-MUC16 chimeric co-stimulatory receptor is a multimer, such as a dimer, trimer, tetramer, pentamer, etc. In some embodiments, the first and second polypeptide chains can be covalently linked, such as by a peptide linkage, or by another chemical linkage, such as a disulfide or other chemical linkage. In some embodiments, the first polypeptide chain and the second polypeptide chain are linked by at least one disulfide bond. In some embodiments, the anti-MUC16 antibody fragment is a Fab, a Fab′, a (Fab′)2, an Fv, or a single chain Fv (scFv).

Examples of co-stimulatory immune cell signaling domains for use in the anti-MUC16 chimeric co-stimulatory receptors include, e.g., cytoplasmic sequences of co-receptors of the T cell receptor (TCR), which can act in concert with a chimeric receptor to initiate signal transduction of the T cell following chimeric receptor engagement, as well as any derivative or variant of these sequences and any synthetic sequence that has the same functionality.

Signals generated through the TCR alone are insufficient for full activation of the T cell, thus requiring a secondary or co-stimulatory signal. T cell activation is said to be mediated by two distinct classes of intracellular signaling sequence: those that initiate antigen-dependent primary activation through the TCR (referred to as “primary immune cell signaling sequences”) and those that act in an antigen-independent manner to provide a secondary or co-stimulatory signal (referred to as “co-stimulatory immune cell signaling sequences”). Primary immune cell signaling sequences that act in a stimulatory manner may contain signaling motifs which are known as immunoreceptor tyrosine-based activation motifs or ITAMs. Examples of ITAM-containing primary immune cell signaling sequences include those derived from TCR FcRy, FcRp, CD3y, CD35, CD3e, CD5, CD22, CD79a, CD79b, and CD66d. A “functional” primary immune cell signaling sequence is a sequence that is capable of transducing an immune cell activation signal when operably coupled to an appropriate receptor. A “non-functional” primary immune cell signaling sequence may include fragments or variants of primary immune cell signaling sequences that are unable to transduce an immune cell activation signal. The anti-MUC16 chimeric co-stimulatory receptors described herein lack a functional primary immune cell signaling sequence, such as a functional signaling sequence comprising an ITAM. In some embodiments, the anti-MUC16 chimeric co-stimulatory receptors lack any primary immune cell signaling sequence. The co-stimulatory immune cell signaling sequence can be a portion of the intracellular domain of a co-stimulatory molecule including, for example, CD27, CD28, 4-IBB (CD137), 0X40, CD30, CD40, PD-1, ICOS, lymphocyte function-associated antigen-1 (LFA-1), CD2, CD7, LIGHT, NKG2C, B7-H3, a ligand that specifically binds with CD83, and the like.

In certain embodiments, the anti-MUC16 antibodies or antigen-binding fragments thereof described herein are capable of inhibiting or reducing metastasis, inhibiting tumor growth, and/or inducing tumor regression in mouse model systems. For example, tumor cell lines can be introduced into athymic nude mice, and the athymic mice can be administered the anti-MUC16 antibodies or antigen-binding fragments thereof of the present invention one or more times, and tumor progression of the injected tumor cells is monitored over a period of weeks and/or months. In some cases, administration of the anti-MUC16 antibodies or antigen-binding fragments thereof to the athymic nude mice can occur prior to the introduction of the tumor cell lines. In a certain embodiment, ovarian cancer cells expressing MUC16-C are used in the mouse xenograft model.

In some embodiments, the anti-MUC16 antibodies or antigen-binding fragment thereof described herein inhibits tumor growth or induces tumor regression in a mouse model by at least about 5%, 10%, 15%, 20%, 25%, 30%, 35%, 40%, 45%, 50%, 55%, 60%, 65%, 70%, 75%, 80%, 85%, 90%, 95%, 98%, 99%, or 100% as assessed by methods described herein or known to one of skill in the art, as compared to placebo-treated mice. In some embodiments, the anti-MUC16 antibodies or antigen-binding fragment thereof inhibits tumor growth or induces tumor regression in a mouse model by at least about 25% or 35%, optionally to about 75%, as assessed by methods described herein or known to one of skill in the art, as compared to mock-treated mice. In some embodiments, the anti-MUC16 antibodies or an antigen-binding fragment thereof described herein inhibit tumor growth or induce tumor regression in a mouse model by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, as compared placebo-treated mice.

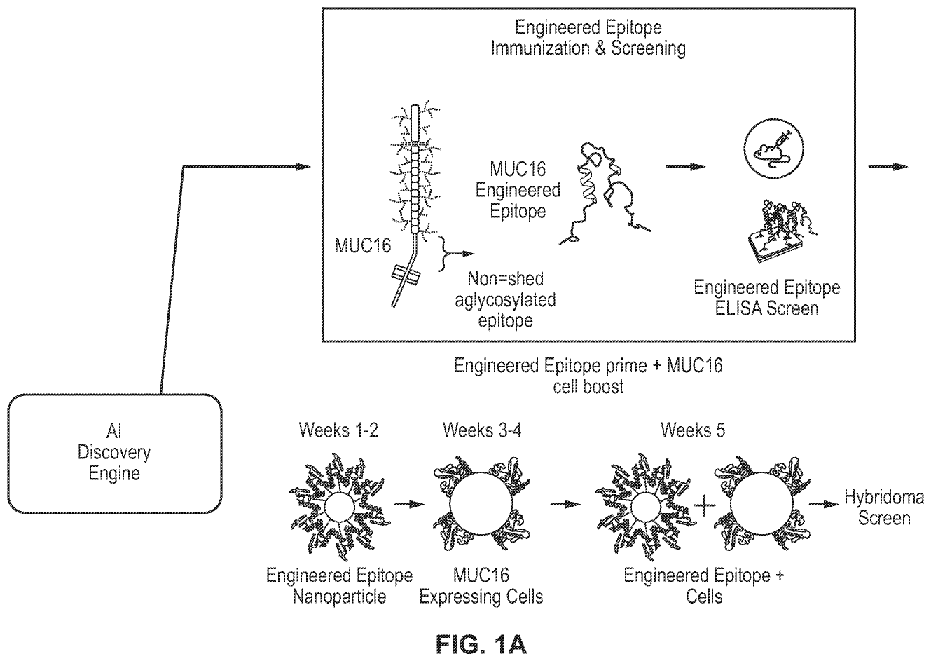

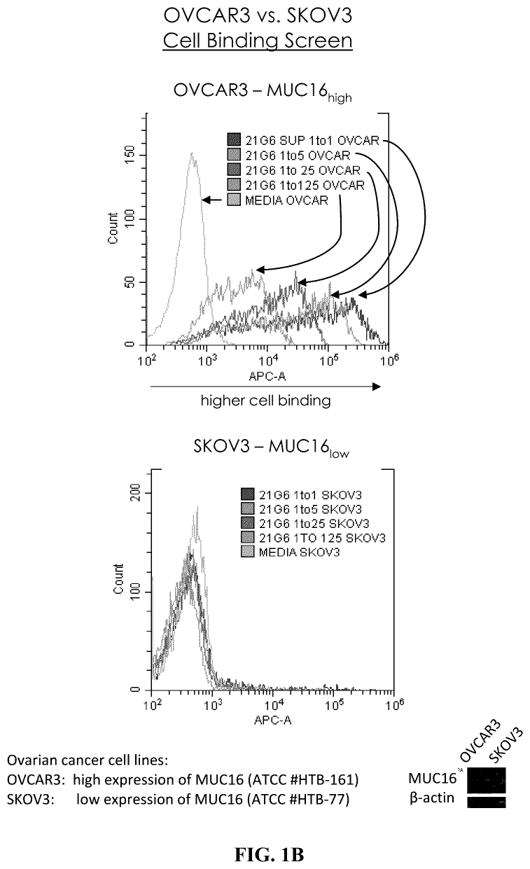

FIG. 1A illustrates the methods for steering immunizations to a MUC16 Epitope that avoids tumor resistance.

Immunization Method: Balb/c mice were injected subcutaneously at multiple sites using an alternating schedule of engineered epitopes conjugated to a KLH or Ferritin nanoparticle and OVCAR-3 cells that express MUC16. The immunization schedule was performed as follows: Day 1: Non-shed 29-mer engineered epitope, Day 5: OVCAR-3 cells, Day 12: OVCAR-3 cells, Day 15: OVCAR-3 cells, Day 19: Non-shed 58-mer glycosylated engineered epitope, Day 22: Non-shed 58-mer glycosylated engineered epitope, Day 27: OVCAR-3 cells, Day 30: Non-shed 58-mer glycosylated engineered epitope. Day 33: OVCAR-3 cells. Splenocytes were collected on Day 36 for hybridoma fusion and subsequent screening.

FIG. 1B are graphs for the cell binding screen that show the results from the immunization strategies by cell binding. The reference western blot shows the relative MUC16 expression on OVCAR-3 and SKOV-3 cells to corroborate the differential cell binding signal.

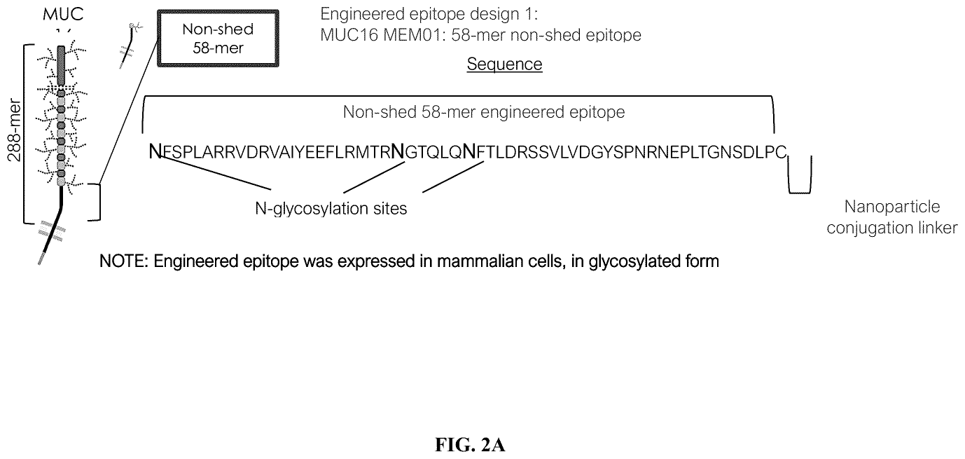

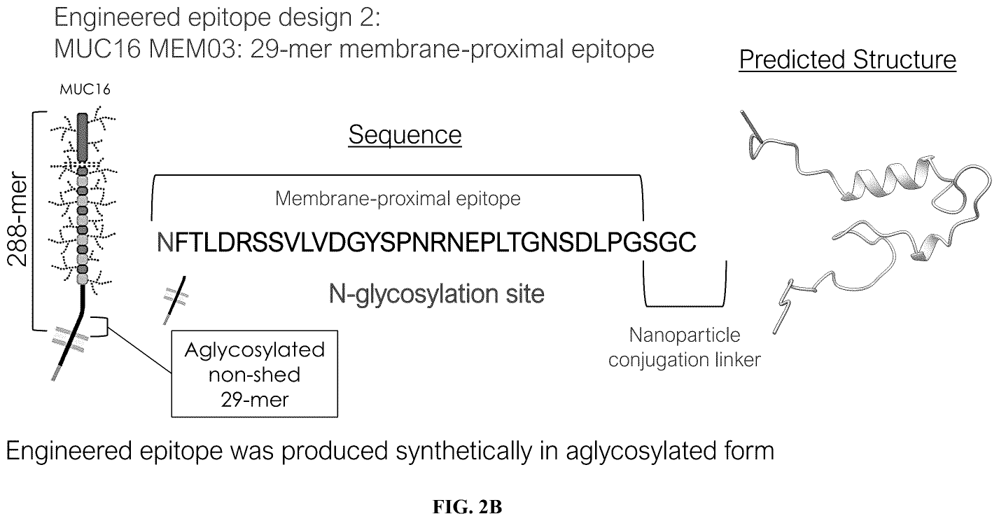

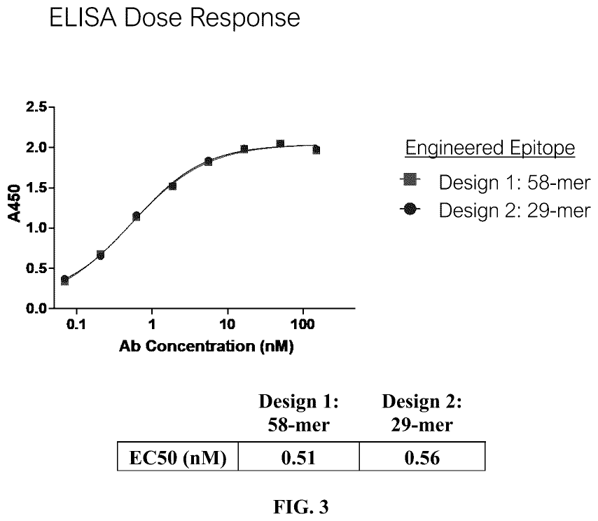

FIG. 2A shows the epitope designed for immunization of engineered epitope 1, which is a 58-mer of MUC16 non-shed domain, including the glycosylation sites. Engineered epitope 1 was expressed in mammalian cells and was used in glycosylated form.

NFSPLARRVDRVAIYEEFLRMTRNGTQLQNFTLDRSSVLVDGYSPNRNEPLTGNSDL PC (SEQ ID NO:373), which was conjugated to a nanoparticle for immunization, in which the bold represents N-glycosylation sites. The engineered epitope was expressed in mammalian cells in glycosylated form.

FIG. 2B shows the epitope designed for immunization of engineered epitope 2, which is a 29-mer of MUC16 non-shed domain, showing the glycosylation site. Engineered epitope 2 was produced synthetically and was used in aglycosylated form.

NFTLDRSSVLVDGYSPNRNEPLTGNSDLPGSGC (SEQ ID NO:374), which was conjugated to a nanoparticle for immunization and in which the bold represents an N-glycosylation sites. The engineered epitope was produced synthetically and is aglycosylated.

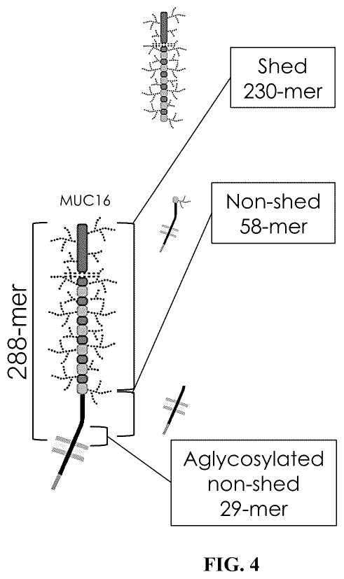

FIG. 4 shows the location of the 29-mer and the non-shed 58-mer in relation to the entire length of MUC16, including the shed domain, the non-shed domain and the location of the aglycosylated non-shed 29-mer and 58-mer in relation to the intracellular and extracellular domains of MUC16.

In some embodiments, the anti-MUC16 antibodies or antigen-binding fragments thereof described herein specifically recognize the peptide epitope of SEQ ID NOS:31 or 32. In some embodiments, the anti-MUC16 antibodies or antigen-binding fragments thereof described herein specifically recognize the retained extracellular domain of human MUC16. In some embodiments, the anti-MUC16 antibodies or antigen-binding fragments thereof described herein specifically bind to the MUC16 ectodomain (MUC16-C). In some embodiments, the anti-MUC16 antibodies or antigen-binding fragments thereof described herein specifically bind to a cell expressing human MUC16, and more particularly, cells that cleave the extracellular domain of MUC16 and express MUC16-C on the cell surface. In some embodiments, the anti-MUC16 antibodies or antigen-binding fragments specifically bind to a cell expressing a recombinant MUC16 polypeptide.

In some embodiments, the anti-MUC16 antibodies or antigen-binding fragments cross-reacts with MUC16 polypeptide from a species other than human. In some embodiments, the anti-MUC16 antibodies or antigen-binding fragments are completely specific for human MUC16 and do not exhibit species or other types of non-human cross-reactivity.

In some embodiments, the anti-MUC16 antibodies or antigen-binding fragments specifically recognize MUC16 expressed on the cell surface of a cancer cell (such as a solid tumor). In some embodiments, the anti-MUC16 antibodies or antigen-binding fragments specifically recognizes MUC16 expressed on the cell surface of ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16. In some embodiments, the anti-MUC16 antibodies and binding fragments thereof specifically recognize MUC16 expressed on the cell surface of a cancer cell line, e.g., ovarian cancer cell lines, such as OVCAR3, OVCA-432, OVCA-433, and CAOV3.

The anti-MUC16 antibodies or antigen-binding fragments in some embodiments is a full-length anti-MUC16 antibodies. In some embodiments, the full-length anti-MUC16 antibody is an IgA, IgD, IgE, IgG, or IgM. In some embodiments, the full-length anti-MUC16 antibody comprises IgG constant domains, such as constant domains of any of IgG1, IgG2, IgG3, and IgG4 including variants thereof. In some embodiments, the full-length anti-MUC16 antibody comprises a lambda light chain constant region. In some embodiments, the full-length anti-MUC16 antibody comprises a kappa light chain constant region. In some embodiments, the full-length anti-MUC16 antibody is a full-length human anti-MUC16 antibody. In some embodiments, the full-length anti-MUC16 antibody comprises an Fc sequence of a mouse immunoglobulin. In some embodiments, the full-length anti-MUC16 antibody comprises an Fc sequence that has been altered or otherwise changed so that it has enhanced antibody-dependent cellular cytotoxicity (ADCC) or complement-dependent cytotoxicity (CDC) effector function.

The skilled artisan will recognize that antibodies which exhibit little or no binding to a target antigen can be described as having a low affinity, and a high equilibrium dissociation constant (KD) for the target antigen. The skilled artisan will also recognize that antibodies which exhibit little or no binding to a collective assembly of target antigenic epitopes can be described as having a low avidity, and a high equilibrium dissociation constant (KD) for the collective assembly of target antigenic epitopes.

In some embodiments, provided herein are anti-MUC16 antibodies having a binding affinity (KD) to MUC16-C of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 PM to about 5 pM.

In some embodiments, anti-MUC16 antibodies have a binding avidity (KD) to MUC16-C of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, anti-MUC16 antibodies have a half maximal effective concentration (EC50) to MUC16-C of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

The skilled artisan will recognize that binding specificity may be determined through a series of competition binding studies, in which a desired antibody demonstrates its ability to prevent binding of a known reference antibody to its target epitope at varying concentrations. In some embodiments, the reference antibody is Ubamatamab. In some embodiments, a reference antibody, binds the epitope known as MUC-16N or MUC16-TR. The skilled artisan will also recognize that isotype-matched antibodies may be used as control antibodies in cell binding assays, ADCC, or CDC assays. In some embodiments, the anti-MUC16-C binding antibodies of the disclosure do not bind to MUC16-N or MUC16-TR.

In some embodiments, the anti-MUC16-C antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of a human antibody is a human IgG1, human IgG2, human IgG3, or human IgG4.

### Exemplary Anti-MUC16 Antibodies—CDR Sequences.

Provided herein are sequences for exemplary anti-MUC16-C antibody antibodies of the disclosure. Included are complementarity determining region (CDR) sequences and the variable heavy and light domain sequences (VH, VL) that constitute the MUC16-C antibody antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure.

TABLE 1
Anti-MUC16-C domain antibody CDRs

| Antibody ID | CDR-H1 | CDR-H2 | CDR-H3 | CDR-L1 | CDR-L2 | CDR-L3 |
|---|---|---|---|---|---|---|
| 1D7 | GFTFSSFA | ISSGGGYI | ARLGGDY | EDIYNR | GAT | QQYWST |
| | | | DEFYAKD | | | WT |
| | | | Y | | | |
| SEQ ID NO: | 2 | 3 | 4 | 7 | 8 | 9 |
| 8G4 | GYTFTNF | INTNTGKP | ARRWDRS | TGAVTTS | GTN | ALWYSN |
| | G | | AWFAY | NY | | HWV |
| SEQ ID NO: | 12 | 13 | 14 | 17 | 18 | 19 |
| 21G6 | GYSITSDY | ISYSGST | ATLGLDY | QSLLYSSN | WAS | QQYHSY |
| | A | | | QKNY | | RT |
| SEQ ID NO: | 22 | 23 | 24 | 27 | 28 | 29 |
| M6I-02-A03 | GFTFSGF | INSDGSAV | MRGTGW | QSLLNSRT | WAS | KQSYNL |
| | W | | YFDV | RKNY | | YT |
| SEQ ID NO: | 32 | 33 | 34 | 37 | 38 | 39 |
| M61-02-C02 | GFTFTDY | IRDKANG | ARDLFGL | SSVSY | DTS | HQWTTY |
| | Y | YTT | YYFDY | | | PYT |
| SEQ ID NO: | 42 | 43 | 44 | 47 | 48 | 49 |
| M61-02-C06 | GFTFSNY | ISSRGYT | AMFYCAR | QSLLDSD | LVS | WQGTHF |
| | A | | ELRLRYF | GKTY | | PRT |
| | | | DV | | | |
| SEQ ID NO: | 52 | 53 | 54 | 57 | 58 | 59 |
| M6I-02-D03 | GYTFTTA | INTHSGVP | ARWQAFD | QNVGTN | SAS | QQYNSY |
| | G | | Y | | | PLT |
| SEQ ID NO: | 62 | 63 | 64 | 67 | 68 | 69 |
| M6I-02-D10 | GFAFNNF | ISRGNTI | GRSYGNY | QSLLDSD | LVS | WQGTHF |
| | G | | VNY | GKTY | | PYT |
| SEQ ID NO: | 72 | 73 | 74 | 77 | 78 | 79 |
| M6I-02-E01 | GFTFTDY | IRNKPNG | ARDNYGL | SSISY | DTS | HQRSSYP |
| | Y | YTT | YFDY | | | WT |
| SEQ ID NO: | 82 | 83 | 84 | 87 | 88 | 89 |
| M61-04-A02 | GYTFTEY | IVPNNGG | ARRDYYG | QSLLNSRT | WAS | KQSYNL |
| | T | T | SSHFDY | RKNY | | YT |
| SEQ ID NO: | 92 | 93 | 94 | 97 | 98 | 99 |
| M61-04-F11 | GYTFTNS | IDPNNDD | TRLLRYQ | ESVDSYG | LAS | QQNNED |
| | Y | T | AWFAY | NSF | | PWT |
| SEQ ID NO: | 102 | 103 | 104 | 197 | 108 | 109 |
| M61-04-H05 | GYTFTEY | IVPNNGG | ARRDYYG | QSLVHSN | KVS | SQSTHVP |
| | T | T | SSHFDY | GNTY | | WT |
| SEQ ID NO: | 112 | 113 | 114 | 117 | 118 | 119 |
| M61-07-A03 | GFTLSSY | ISPAGGGT | ASPLYPY | QDISIY | HAS | QQNYGT |
| | W | | GLDI | | | PYT |
| SEQ ID NO: | 122 | 123 | 124 | 127 | 128 | 129 |
| M61-07-A05 | GFTFSNY | ISSGGGG | YCASPPR | ENIGDY | AAS | QQNYDF |
| | A | | DYEGMD | | | PLT |
| | | | V | | | |
| SEQ ID NO: | 132 | 133 | 134 | 137 | 138 | 139 |
| M61-07-B05 | GFTFSNY | ISSGGGG | YCASPPR | QDISIY | TAS | QQSYSTL |
| | A | | DYEGMD | | | T |
| | | | V | | | |
| SEQ ID NO: | 142 | 143 | 144 | 147 | 148 | 149 |
| M6I-07-C06 | GFTFSNY | ISSGGGG | YCASPPR | QQISTY | ATS | QQTYITP |
| | A | | DYEGMDV | | | RT |
| SEQ ID NO: | 152 | 153 | 154 | 157 | 158 | 159 |
| M6I-07-D05 | GFPFSHY | ISGSGHTT | ARARFPP | HSIGAY | AAS | QQSYSTP |
| | A | | YYLDI | | | K |
| SEQ ID NO: | 162 | 163 | 164 | 167 | 168 | 169 |
| M6I-07-E01 | GFTFSNY | ISSGGGG | YCASPPR | QQISTY | ATS | QQTYITP |
| | A | | DYEGMD | | | RT |
| | | | V | | | |
| SEQ ID NO: | 172 | 173 | 174 | 177 | 178 | 179 |
| M6I-07-F05 | GFTFSNY | ISSGGGG | YCASPPR | QQISTY | ATS | QQTYITP |
| | A | | DYEGMD | | | RT |
| | | | V | | | |
| SEQ ID NO: | 182 | 183 | 184 | 187 | 188 | 189 |
| M6I-07-F06 | GFTFSNY | ISGRGGHT | ASPPIRDA | QSVNRL | AAS | QQSYSIP |
| | A | | GMDV | | | HT |
| SEQ ID NO: | 192 | 193 | 194 | 197 | 198 | 199 |
| M6I-07-G03 | GFTFSNY | ITGPGGST | ARDLYGF | QSIGTH | AAS | QQTYITP |
| | V | | DV | | | RT |
| SEQ ID NO: | 202 | 203 | 204 | 207 | 208 | 209 |
| M6I-07-H01 | GFNFANY | VSGSGDY | ARDFWW | QTIKNY | AAS | QQSYSIP |
| | A | T | GLEGMDY | | | YS |
| SEQ ID NO: | 212 | 213 | 214 | 217 | 218 | 219 |
| M61-07-H04 | GFTFSNY | ISSGGGG | YCASPPR | QQISTY | ATS | QQTYITP |
| | A | | DYEGMD | | | RT |
| | | | V | | | |
| SEQ ID NO: | 222 | 223 | 224 | 227 | 228 | 229 |
| M61-07-H05 | GFSLSDY | IRASDEST | ASGSGAL | QTIKNY | AAS | QQSYSTP |
| | D | | YDPFDV | | | PT |
| SEQ ID NO: | 232 | 233 | 234 | 237 | 238 | 239 |
| M61-07-H06 | GFTFSNY | ISSGGGG | YCASPPR | QQISTY | ATS | QQTYITP |
| | A | | DYEGMD | | | RT |
| | | | V | | | |
| SEQ ID NO: | 242 | 243 | 244 | 247 | 248 | 249 |
| M61-08-A05 | GYSITSDY | ISYSGST | ATLGLDY | QSLLYSSN | WAS | QQYHSY |
| | A | | | QKNY | | RT |
| SEQ ID NO: | 252 | 253 | 254 | 257 | 258 | 259 |
| M6I-08-B05 | GFTFSTYA | ISDGGRYT | ARRDGFF | QNVGTA | SAS | KQSYNL |
| | | | FDS | | | YT |
| SEQ ID NO: | 262 | 263 | 264 | 267 | 268 | 269 |
| M61-09-A08 | GYTFTSY | INPSNGRT | ANWDY | QNVGTN | WAS | QQYSSYP |
| | W | | | | | LT |
| SEQ ID NO: | 272 | 273 | 274 | 277 | 278 | 279 |
| M61-09-B03 | GYTFTNS | IDPNNDD | TRLLRYQ | QNVGTN | SAS | QQYNSY |
| | Y | T | AWFAY | | | PYT |
| SEQ ID NO: | 282 | 283 | 284 | 287 | 288 | 289 |
| M6I-09-D05 | GYTFTNY | INPSNGGT | VYDGYYR | ESVDSYG | LAS | QQNNED |
| | Y | | DRYFDV | NSF | | PWT |
| SEQ ID NO: | 292 | 293 | 294 | 297 | 298 | 299 |
| M6I-09-F08 | GFTFSTYA | ISDGGRYT | ARRDGFF | QNVGTN | SAS | QQWSSN |
| | | | FDS | | | PPIT |
| SEQ ID NO: | 302 | 303 | 304 | 307 | 308 | 309 |
| M6I-10-D03 | GYTFTDY | INTATGDS | ARTTF | QNVGTN | SAS | QQYNSY |
| | S | | | | | PLT |
| SEQ ID NO: | 312 | 313 | 314 | 317 | 318 | 319 |
| M6I-10-D10 | GYTFTNS | IDPNNDD | TRLLRYQ | ESVDSYG | LAS | QQNNED |
| | Y | T | AWFAY | NSF | | PWT |
| SEQ ID NO: | 322 | 323 | 324 | 327 | 328 | 329 |
| M61-11-D08 | GFTFSSFG | ISSGGNYT | ARDDRYD | SSVSY | DTS | QQYSSYP |
| | | | DFDY | | | YT |
| SEQ ID NO: | 332 | 333 | 334 | 337 | 338 | 339 |
| M6I-11-D10 | GFAFSTFG | ISSGGNYT | ARDDRYD | SSVSY | DTS | QQWSSN |
| | | | DFDY | | | PPT |
| SEQ ID NO: | 342 | 343 | 344 | 347 | 348 | 349 |
| M6I_02-G04 | GFTFTDY | IRNKANG | ARDYYGS | SSVSY | DTS | QQWSSN |
| | Y | YTT | LFAY | | | PPT |
| SEQ ID NO: | 352 | 353 | 354 | 357 | 358 | 359 |

TABLE 2
Clones VH, VL Amino Acid

| | | | SEQ |
|---|---|---|---|
| Clone | | | ID |
| ID | Chain | Amino Acid Sequence | NO: |
| 1D7 | VH | EVMLVESGGGLVKPGGSLKLSCVAS | 1 |
| | | GFTFSSFAMSWVRQTPEKRLEWVAI | |
| | | ISSGGGYIYYADSMTGRFTISRDNA | |
| | | KNTLYLQMSSLRSEDTAMYYCARLG | |
| | | GDYDEFYAKDYWGQGTSVTVSS | |
| 1D7 | VL | DIQMTQSSSSFSVSLGDRVTITCKA | 6 |
| | | SEDIYNRLAWYQQKPGNAPRLLISG | |
| | | ATSLETGVPSRFSGSGSGKDYTLSI | |
| | | TSLQTEDVATYYCQQYWSTWTFGGG | |
| | | TKLEIK | |
| 8G4 | VH | QIQLVQSGPELKMPGETVKISCKAS | 11 |
| | | GYTFTNFGMNWVKQAPGKALKWMGW | |
| | | INTNTGKPTYFEEFKGRFAFSLETS | |
| | | ASTAYLQINNLKNEDTATYFCARRW | |
| | | DRSAWFAYWGQGTLVTVSA | |
| 8G4 | VL | QAVVTQESALTTSPGETVTLTCRSS | 16 |
| | | TGAVTTSNYANWVQEKPDHLFTGLI | |
| | | GGTNDRSPGVPARFSGSLIGDKAAL | |
| | | TITGAQTEDEAIYFCALWYSNHWVF | |
| | | GGGTKLTVL | |
| 21G6 | VH | EVQLEESGPGLVKPSQSLSLTCTVT | 21 |
| | | GYSITSDYAWNWIRQFPGNKLEWMG | |
| | | YISYSGSTIYNPSLKSRISITRDTS | |
| | | KNQFFLHLNSVTTEDTATYYCATLG | |
| | | LDYWGQGTTLTVSS | |
| 21G6 | VL | DFVMTQSPSSLAVSVGEKVTMSCKS | 26 |
| | | SQSLLYSSNQKNYLAWYQQKPGQSP | |
| | | KLLIYWASTRGSGVPDRFTGSGSGT | |
| | | DFTLTISSVKAEDLAVYYCQQYHSY | |
| | | RTFGGGTKLEIK | |
| M6I-02-A03 | VH | EVQLLETGGGLVRPGGSRGLSCEGS | 31 |
| | | GFTFSGFWMSWVRQTPGKTLEWIGD | |
| | | INSDGSAVNYAPSIKDRFTIFRDND | |
| | | KSTLYLQMSNVRSEDTATYFCMRGT | |
| | | GWYFDVWGAGTTVTVSS | |
| | VL | DIVMTQSPSSLAVSAGEKVTMSCKS | 36 |
| | | SQSLLNSRTRKNYLAWYQQKPGQSP | |
| | | KLLIYWASTRESGVPDRFTGSGSGT | |
| | | DFTLTISSVQAEDLAVYYCKQSYNL | |
| | | YTFGGGTKLEIK | |
| M6I-02-C02 | VH | DVMLVESGGGLVQPGASLRLSCATL | 41 |
| | | GFTFTDYYMSWVRQPPGKALEWLGF | |
| | | IRDKANGYTTEYSASVKGRFTISRD | |
| | | NSQSILYLQMNTLRAEDSATYYCAR | |
| | | DLFGLYYFDYWGQGTTLTVSS | |
| | VL | DIVLTQSPAIMSASPGEKVTMTCSA | 46 |
| | | SSSVSYMHWYQQKSGTSPKRWIYDT | |
| | | SKLASGVPARFSGSGSGTSYSLTIS | |
| | | SMEAEDAATYFCHQWTTYPYTFGGG | |
| | | TKLEIK | |
| M6I-02-C06 | VH | DVQLVESGGGLVKPGGSLKLSCAAS | 51 |
| | | GFTFSNYAMSWVRQTPEKRLEWVAS | |
| | | ISSRGYTYYADSVKADSVKGRFTIS | |
| | | RDNARNILYLQMSSLRSEDTAMFYC | |
| | | ARELRLRYFDVWGAGTTVTVSS | |
| | VL | DVVLTQTPLTLSVTIGQPASISCKS | 56 |
| | | SQSLLDSDGKTYLNWLLQRPGQSPK | |
| | | RLIHLVSKLDSGVPDRFTGSGSGTD | |
| | | FTLKISRVEAEDLGVYYCWQGTHFP | |
| | | RTFGGGTKLEIK | |
| M6I-02-D03 | VH | QIQLVQSGPELKKPGETVRISCKAS | 61 |
| | | GYTFTTAGMQWVQKMPGKGLKWIGW | |
| | | INTHSGVPKYAEDFKGRFAFSLETS | |
| | | ASTAYLQISNLKNEDTATYFCARWQ | |
| | | AFDYWGQGTTVTVSS | |
| | VL | DIVMTQSQKFMSTSVGDRVSVTCKA | 66 |
| | | SQNVGTNVAWYQQKPGQSPKALIYS | |
| | | ASYRYSGVPDRFTGSGSGTDFTLTI | |
| | | SNVQSEDLADYFCQQYNSYPLTFGG | |
| | | GTKLEIK | |
| M6I-02-D10 | VH | DVKLVESGGGLVQPGGSRKLSCAAS | 71 |
| | | GFAFNNFGIYWVRQAPEKGLEWVAY | |
| | | ISRGNTIYYADTVKGRFTISRDNPK | |
| | | NTLFLQMTSLRSEDTAMYYCGRSYG | |
| | | NYVNYWGLGTTLTVSS | |
| | VL | DIVMTQTPLTLSVTIGQPASISCKS | 76 |
| | | SQSLLDSDGKTYLNWLLQRPGQSPK | |
| | | RLIYLVSKLDSGVPDRFTGSGSGTD | |
| | | FTLKISRVEAEDLGVYYCWQGTHFP | |
| | | YTFGGGTKLEIK | |
| M6I-02-E01 | VH | EVMLVESGGGLVQPGGSLRLSCATS | 81 |
| | | GFTFTDYYMSWVRQPPGKALEWLGF | |
| | | IRNKPNGYTTEYSASVKGRFTISRD | |
| | | NSQSILYLQMNTLRAEDSATYYCAR | |
| | | DNYGLYFDYWGQGTTLTVSS | |
| | VL | DIVLTQSPAIMSASPGEKVTMTCSA | 86 |
| | | SSSISYMHWYQQKPGTSPKRWIYDT | |
| | | SKLASGVPARFSGSGSGTSYSLTIS | |
| | | SMEAEDAATYYCHQRSSYPWTFGGG | |
| | | TKLEIK | |
| M6I-04-A02 | VH | EVRLQQSGPELVKPGASVKISCKTS | 91 |
| | | GYTFTEYTMHWVKQSHGKSLEWIGG | |
| | | IVPNNGGTNYNQKFKVKATLTVDKS | |
| | | SSTAYMELRSLTSEDSAVYYCARRD | |
| | | YYGSSHFDYWGQGTTLTVSS | |
| | VL | DILMTQSPSSLAVSVGEKVTMSCKS | 96 |
| | | SQSLLNSRTRKNYLAWYQQKPGQSP | |
| | | KLLIYWASTRESGVPDRFTGSGSGT | |
| | | DFTLTISNVQAEDLAVYYCKQSYNL | |
| | | YTFGGGTKLEIK | |
| M6I-04-F11 | VH | QVQLQQPGAELVKPGASVKLSCKAS | 101 |
| | | GYTFTNSYIYWVKQRPGQGLEWIGG | |
| | | IDPNNDDTNFNEEFKSKATLTVDKS | |
| | | SSTAYMQLSSLTSEDSAVYYCTRLL | |
| | | RYQAWFAYWGQGTLVTVSS | |
| | VL | DIVLTQSPASLAVSLGQRATISCRA | 106 |
| | | SESVDSYGNSFMHWYQQKPGQPPKL | |
| | | LIYLASNLESGVPARFSGSGSRTDF | |
| | | TLTIDPVEADDAATYYCQQNNEDPW | |
| | | TFGGGTKLEIK | |
| M6I-04-H05 | VH | EVRLQQSGPELVKPGASVKISCKTS | 111 |
| | | GYTFTEYTMHWVKQSHGKSLEWIGG | |
| | | IVPNNGGTNYNQKFKVKATLTVDKS | |
| | | SSTAYMELRSLTSEDSAVYYCARRD | |
| | | YYGSSHFDYWGQGTTLTVSS | |
| | VL | DVVMTQTPLSLPVSLGDQASISCRS | 116 |
| | | SQSLVHSNGNTYLHWYLQKPGQSPK | |
| | | LLIYKVSNRFSGVPDRFSGSGSGTD | |
| | | FTLKISRVEAEDLGVYFCSQSTHVP | |
| | | WTFGGGTKLEIK | |
| M6I-07-A03 | VH | EVQLLESGGGLVQPGGSLRLSCAAS | 121 |
| | | GFTLSSYWMHWVRQAPGKGLEWVSA | |
| | | ISPAGGGTYYADSVKGRFTISRDNS | |
| | | KNTLYLQMNSLRAEDTAVYYCASPL | |
| | | YPYGLDIWGQGTLVTVSS | |
| | VL | DIQMTQSPSSLSASVGDRVTITCRA | 126 |
| | | SQDISIYLNWYQQKPGKAPKLLIYH | |
| | | ASNLQSGVPSRFSGSGSGTDFTLTI | |
| | | SSLQPEDFATYYCQQNYGTPYTFGG | |
| | | GTKVEIK | |
| M6I-07-A05 | VH | EVQLLESGGGLVQPGGSLRLSCAAS | 131 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 136 |
| | | SENIGDYLSWYQQKPGKAPKLLIYA | |
| | | ASSLQSGVPSRFSGSGSGTDFTLTI | |
| | | SSLQPEDFATYYCQQNYDFPLTFGG | |
| | | GTKVEIK | |
| M6I-07-B05 | VH | EVQLLESGGGLVQPGGSLRLSCAAS | 141 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 146 |
| | | SQDISIYLNWYQQKPGKAPKLLIYT | |
| | | ASNLQSGVPSRFSGSGSGTDFTLTI | |
| | | SSLQPEDFATYYCQQSYSTLTFGGG | |
| | | TKVEIK | |
| M6I-07-C06 | VH | EVQLLESGGGLVQPGGSLRLSCAAS | 151 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 156 |
| | | SQQISTYLNWYQQKPGKAPKLLIYA | |
| | | TSRLEDGVPSRFSGSGSGTDFTLTI | |
| | | SSLQPEDFATYYCQQTYITPRTFGG | |
| | | GTKVEIK | |
| M6I-07-D05 | VH | EVQLLESGGGLVQPGGSLRLSCAAS | 161 |
| | | GFPFSHYAMSWVRQAPGKGLEWVSG | |
| | | ISGSGHTTDYADSVKGRFTISRDNS | |
| | | KNTLYLQMNSLRAEDTAVYYCARAR | |
| | | FPPYYLDIWGQGTLVTVSS | |
| | VL | DIQMTQSPSSLSASVGDRVTITCRA | 166 |
| | | SHSIGAYLNWYQQKPGKAPKLLIYA | |
| | | ASSLQSGVPSRFSGSGSGTDFTLTI | |
| | | SSLQPEDFATYYCQQSYSTPKFGGG | |
| | | TKVEIK | |
| M6I-07-E01 | VH | EVQLLESGGGLVQPGGSLRLSCAAS | 171 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 176 |
| | | SQQISTYLNWYQQKPGKAPKLLIYA | |
| | | TSRLEDGVPSRFSGSGSGTDFTLTI | |
| | | SSLQPEDFATYYCQQTYITPRTFGG | |
| | | GTKVEIK | |
| M6I-07-F05 | VH | EVQLLESGGGLVQPGGSLRLSCAAS | 181 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 186 |
| | | SQQISTYLNWYQQKPGKAPKLLIYA | |
| | | TSRLEDGVPSRFSGSGSGTDFTLTI | |
| | | SSLQPEDFATYYCQQTYITPRTFGG | |
| | | GTKVEIK | |
| M6I-07-F06 | VH | EVQLLESGGGLVQPGGSLRLSCAAS | 191 |
| | | GFTFSNYAMGWVRQAPGKGLEWVSV | |
| | | ISGRGGHTYYADSVKGRFTISRDNS | |
| | | KNTLYLQMNSLRAEDTAVYYCASPP | |
| | | IRDAGMDVWGQGTLVTVSS | |
| | VL | DIQMTQSPSSLSASVGDRVTITCRA | 196 |
| | | SQSVNRLLNWYQQKPGKAPKLLIYA | |
| | | ASSLQSGVPSRFSGSGSGTDFTLTI | |
| | | SSLQPEDFATYYCQQSYSIPHTFGG | |
| | | GTKVEIK | |
| M6I-07-G03 | VH | EVQLLESGGGLVQPGGSLRLSCAAS | 201 |
| | | GFTFSNYVMGWVRQAPGKGLEWVST | |
| | | ITGPGGSTYYADSVKGRFTISRDNS | |
| | | KNTLYLQMNSLRAEDTAVYYCARDL | |
| | | YGFDVWGQGTLVTVSS | |
| | VL | DIQMTQSPSSLSASVGDRVTITCRA | 206 |
| | | SQSIGTHVNWYQQKPGKAPKLLIYA | |
| | | ASSLQSGVPSRFSGSGSGTDFTLTI | |
| | | SSLQPEDFATYYCQQTYITPRTFGG | |
| | | GTKVEIK | |
| M6I-07-H01 | VH | EVQLLESGGGLVQPGGSLRLSCAAS | 211 |
| | | GENFANYAMNWVRQAPGKGLEWVSA | |
| | | VSGSGDYTHYADSVKGRFTISRDNS | |
| | | KNTLYLQMNSLRAEDTAVYYCARDF | |
| | | WWGLEGMDYWGQGTLVTVSS | |
| | VL | DIQMTQSPSSLSASVGDRVTITCRA | 216 |
| | | SQTIKNYVNWYQQKPGKAPKLLIYA | |
| | | ASNFQSGVPSRFSGSGSGTDFTLTI | |
| | | SSLQPEDFATYYCQQSYSIPYSFGG | |
| | | GTKVEIK | |
| M6I-07-H04 | VH | EVQLLESGGGLVQPGGSLRLSCAAS | 221 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 226 |
| | | SQQISTYLNWYQQKPGKAPKLLIYA | |
| | | TSRLEDGVPSRFSGSGSGTDFTLTI | |
| | | SSLQPEDFATYYCQQTYITPRTFGG | |
| | | GTKVEIK | |
| M6I-07-H05 | VH | EVQLLESGGGLVQPGGSLRLSCAAS | |
| | | GFSLSDYDMTWVRQAPGKGLEWVST | |
| | | IRASDESTYYADSVKGRFTISRDNS | |
| | | KNTLYLQMNSLRAEDTAVYYCASGS | |
| | | GALYDPFDVWGQGTLVTVSS | 231 |
| | VL | DIQMTQSPSSLSASVGDRVTITCRA | 236 |
| | | SQTIKNYVNWYQQKPGKAPKLLIYA | |
| | | ASNFQSGVPSRFSGSGSGTDFTLTI | |
| | | SSLQPEDFATYYCQQSYSTPPTFGG | |
| | | GTKVEIK | |
| M6I-07-H06 | VH | EVQLLESGGGLVQPGGSLRLSCAAS | 241 |
| | | GFTFSNYAMGWVRQAPGKGLEWVSV | |
| | | ISSGGGGGTSYADSVKGRFTISRDN | |
| | | SKNTLYLQMNSLRAEDTAVYYCASP | |
| | | PRDYEGMDVWGQGTLVTVSS | |
| | VL | DIQMTQSPSSLSASVGDRVTITCRA | 246 |
| | | SQQISTYLNWYQQKPGKAPKLLIYA | |
| | | TSNLHGGVPSRFSGSGSGTDFTLTI | |
| | | SSLQPEDFATYYCQQTYITPRTFGG | |
| | | GTKVEIK | |
| M6I-08-A05 | VH | QVQLQESGPGLVKPSQTLSLTCTVS | 251 |
| | | GYSITSDYAWNWIRQPPGKGLEWIG | |
| | | YISYSGSTIYNPSLKSRISISVDTS | |
| | | KNQFSLKLNSVTAADTAVYYCATLG | |
| | | LDYWGQGTLVTVSS | |
| | VL | DIVMTQSPDSLAVSLGERATINCKS | 256 |
| | | SQSLLYSSNQKNYLAWYQQKPGQPP | |
| | | KLLIYWASTRGSGVPDRFSGSGSGT | |
| | | DFTLTISSLQAEDVAVYYCQQYHSY | |
| | | RTFGQGTKVEIK | |
| M6I-08-B05 | VH | EVKLVESGGGLVKPGGSLKLSCAAS | 261 |
| | | GFTFSTYAMSWVRQTPEKRLEWVAT | |
| | | ISDGGRYTYYPDSVKGRFTISRDNA | |
| | | KNNLYLQMSSLKSEDTAMYYCARRD | |
| | | GFFFDSWGQGTTLTVSS | |
| | VL | DTTVTQSQKFMSTSVGDRVSITCKA | 266 |
| | | SQNVGTAVAWYQQKPGQSPKLLIYS | |
| | | ASNRYTGVPDRFTGSGSGTDFTLTI | |
| | | SSVQAEDLAVYYCKQSYNLYTFGGG | |
| | | TKLEMK | |
| M6I-09-A08 | VH | QVQLQQPGAELVKPGASVKLSCKAS | 271 |
| | | GYTFTSYWMHWVKQRPGQGLEWIGE | |
| | | INPSNGRTNYNEKFKSKATLTVDKS | |
| | | SSTAYMQLSSLTSEDSAVYYCANWD | |
| | | YWGQGTTLTVSS | |
| | VL | DVVMTQSQKFLSTSVGDRVGVTCKA | 276 |
| | | SQNVGTNVAWYQQKPGQSPKLLIYW | |
| | | ASTRHTGVPDRFTGSGSGTDFTLTI | |
| | | SNVQSEDLADYFCQQYSSYPLTFGG | |
| | | GTKLEMK | |
| M6I-09-B03 | VH | QVQLKESGAELVKPGASVKLSCKAS | 281 |
| | | GYTFTNSYIYWVKQRPGQGLEWIGG | |
| | | IDPNNDDTNFNEEFKSKATLTVDKS | |
| | | SSTAYMQLSSLTSEDSAVYYCTRLL | |
| | | RYQAWFAYWGQGTLVTVSS | |
| | VL | DVVMTQSQKFMSTSVGDRVSVTCKA | 286 |
| | | SQNVGTNVAWYQQKPGQSPKALIYS | |
| | | ASYRYSGVPDRFTGSGSGTDFTLTI | |
| | | SNVQSEDLAEYFCQQYNSYPYTFGG | |
| | | GTKLEIK | |
| M6I-09-D05 | VH | QVQLQQPGAELVKPGASVKLSCKAS | 291 |
| | | GYTFTNYYIYWVKQRPGQGLEWIGE | |
| | | INPSNGGTHFNEKFESKATLTVDKS | |
| | | SSTTYMQLNSLTSEDSAVYYCVYDG | |
| | | YYRDRYFDVWGAGTTVTVSS | |
| | VL | DIVLTQSPASLAVSLGQRATISCRA | 296 |
| | | SESVDSYGNSFMHWYQQKPGQPPKL | |
| | | LIYLASNLESGVPARFSGSGSRTDF | |
| | | TLTIDPVEADDAATYYCQQNNEDPW | |
| | | TFGGGTKLEIK | |
| M6I-09-F08 | VH | EVKLVESGGGLVQPGGSRKLSCAAS | 301 |
| | | GFTFSTYAMSWVRQTPEKRLEWVAT | |
| | | ISDGGRYTYYPDSVKGRFTISRDNA | |
| | | KNNLYLQMSSLKSEDTAMYYCARRD | |
| | | GFFFDSWGQGTTLTVSS | |
| | VL | DIVMTQSQKFMSTSVGDRVSVTCKA | 306 |
| | | SQNVGTNVAWYQQKPGQSPKALIYS | |
| | | ASYRYSGVPDRFTGSGSGTDFTLTI | |
| | | SSMEAEDAAIYYCQQWSSNPPITFG | |
| | | GGTKLEMK | |
| M6I-10-D03 | VH | QVQLQQSGPELKKPGETVRISCKAS | 311 |
| | | GYTFTDYSIYWVKQAPGQGLKLMGW | |
| | | INTATGDSTYADDFQGRFAFSLETS | |
| | | ASTAYLQINNLKNDDTATYFCARTT | |
| | | FWGQGTTLTVSS | |
| | VL | DVVMTQSHKFMSTSVGDRVSVTCKA | 316 |
| | | SQNVGTNVAWYQQKPGQSPKALIYS | |
| | | ASYRYSGVPDRFTGSGSGTDFTLTI | |
| | | SNVQSEDLADYFCQQYNSYPLTFGG | |
| | | GTKLEMK | |
| M6I-10-D10 | VH | QVQLQQPGAELVKPGASVKLSCKAS | 321 |
| | | GYTFTNSYIYWVKQRPGQGLEWIGG | |
| | | IDPNNDDTNFNEEFKSKATLTVDKS | |
| | | SSTAYMQLSSLTSEDSAVYYCTRLL | |
| | | RYQAWFAYWGQGTLVTVSS | |
| | VL | DIVLTQSPASLAVSLGQRATISCRA | 326 |
| | | SESVDSYGNSFMHWYQQKPGQPPKL | |
| | | LIYLASNLESGVPARFSGSGSRTDF | |
| | | TLTIDPVEADDAATYYCQQNNEDPW | |
| | | TFGGGTKLEIK | |
| M6I-11-D08 | VH | EVKLVESGGGLVKPGGSLKLSCAAS | 331 |
| | | GFTFSSFGMHWVRQAPEKGLEWVAY | |
| | | ISSGGNYTYYSDSVKGRFTVSRDNA | |
| | | KNTLFLQMSSLRSEDTAMYYCARDD | |
| | | RYDDFDYWGQGTTLTVSS | |
| | VL | DTTVTQSPAIMSASPGEKVTMTCSA | 336 |
| | | SSSVSYMYWYQQKPGSSPRLLIYDT | |
| | | SNLASGVPARFSGSGSGTSYSLTIS | |
| | | SMEAEDAATYYCQQYSSYPYTFGGG | |
| | | TKLEIK | |
| M6I-11-D10 | VH | EVQLVESGGGLVQPGGSRKLSCAAS | 341 |
| | | GFAFSTFGMHWIRQAPEKGLEWVAS | |
| | | ISSGGNYTYYSDSVKGRFTVSRDNA | |
| | | KNTLFLQMSSLRSEDTAMYYCARDD | |
| | | RYDDFDYWGQGTTLTVSS | |
| | VL | DTTVTQSPAIMSASPGEKVTMTCSA | 346 |
| | | SSSVSYMYWYQQKPGSSPRLLIYDT | |
| | | SNLASGVPARFSGSGSGTSYSLTIS | |
| | | SMEAEDAATYYCQQWSSNPPTFGGG | |
| | | TKLEIK | |
| M6I02-G04 | VH | EVKLVESGGGLVQPGGSLRLSCTTS | 351 |
| | | GFTFTDYYMSWVRQPPGKALEWLGF | |
| | | IRNKANGYTTEYSTSVKGRFTISRD | |
| | | NSHSILYLQMNTLRAEDSATYYCAR | |
| | | DYYGSLFAYWGQGTLVTVSA | |
| | VL | DNVLTQSPALMSASPGEKVTMTCSA | 356 |
| | | SSSVSYMHWYQQKSGTSPKRWIYDT | |
| | | SKLASGVPARFSGSGSGTSYSLTIS | |
| | | SMEAEDAATYYCQQWSSNPPTFGGG | |
| | | TKLEIK | |
| Hum1D7 | VH | EVQLVESGGGLVKPGGSLRLSCVAS | 361 |
| | | GFTFSSFAMSWVRQAPGKGLEWVAI | |
| | | ISSGGGYIYYADSMTGRFTISRDNA | |
| | | KNSLYLQMSSLRAEDTAVYYCARLG | |
| | | GDYDEFYAKDYWGQGTLVTVSS | |
| | VH | EVQLVESGGGLVKPGGSLRLSCAAS | 363 |
| | | GFTFSSFAMSWVRQAPGKGLEWVSI | |
| | | ISSGGGYIYYADSMTGRFTISRDNA | |
| | | KNSLYLQMNSLRAEDTAVYYCARLG | |
| | | GDYDEFYAKDYWGQGTLVTVSS | |
| Hum1D7 | VH | EVQLVESGGGLVQPGGSLRLSCAAS | 365 |
| | | GFTFSSFAMSWVRQAPGKGLEWVSI | |
| | | ISSGGGYIYYADSMTGRFTISRDNS | |
| | | KNTLYLQMNSLRAEDTAVYYCAKLG | |
| | | GDYDEFYAKDYWGQGTMVTVSS | |
| | VL | DIQMTQSPSSLSASVGDRVTITCKA | 367 |
| | | SEDIYNRLAWYQQKPGKAPRLLIYG | |
| | | ATSLETGVPSRFSGSGSGTDFTLTI | |
| | | TSLQPEDVATYYCQQYWSTWTFGQG | |
| | | TKVEIK | |
| Hum1D7 | VL | DIQMTQSPSSLSASVGDRVTITCKA | 369 |
| | | SEDIYNRLAWYQQKPGKAPKLLIYG | |
| | | ATSLETGVPSRFSGSGSGTDFTLTI | |
| | | SSLQPEDVATYYCQQYWSTWTFGQG | |
| | | TKVEIK | |
| | VL | DIQMTQSPSSLSASVGDRVTITCKA | 371 |
| | | SEDIYNRLAWYQQKPGKAPKLLIYG | |
| | | ATSLETGVPSRFSGSGSGTDFTLTI | |
| | | SSLQPEDFATYYCQQYWSTWTFGQG | |
| | | TKVEIK | |

TABLE 3
Clones VH, VL Nucleic Acid

| | | | SEQ |
|---|---|---|---|
| Clone | | | ID |
| ID | Chain | Nucleotide sequence | NO: |
| 1D7 | VH | gaagttatgttagtcgaaagtggag | 5 |
| | | ggggctagtgaagcctgggggtagt | |
| | | ctgaaactctcctgcgtagcttcag | |
| | | gattcaccttctcgtcatttgcaat | |
| | | gagctgggtcagacagacccccgag | |
| | | aagcggcttgaatgggggctattat | |
| | | aagctccggggggggtacatctatt | |
| | | atgccgactccatgaccggacgctt | |
| | | tacaatctctagggacaatgcaaag | |
| | | aacacgttgtacctgcaaatgtcca | |
| | | gcctccgaagcgaggatacagccat | |
| | | gtactattgtgcccgtctgggcggt | |
| | | gactacgatgagttctacgccaaag | |
| | | attattggggccagggcacttcagt | |
| | | gactgtgtcttc | |
| 1D7 | VL | acatacagatgacccaatctagcag | 10 |
| | | ttcgttcagcgtgtcacttggtgat | |
| | | cgcgtgactatcacatgtaaggcta | |
| | | gtgaggacatttataaccgactggc | |
| | | ctggtatcagcagaagcccggcaat | |
| | | gcaccacggctgttaatctctggag | |
| | | ctacttccttggaaacaggagtccc | |
| | | ttccaggttctccgggagcggcagc | |
| | | gggaaggactacaccctgtctatca | |
| | | catcccttcagacagaggatgttgc | |
| | | cacctactattgccagcaatactgg | |
| | | tcaacttggaccttcggaggcggta | |
| | | cgaaactcgaaattaa | |
| 8G4 | VH | cagatccagttggtgcagtctggac | 15 |
| | | ctgagctgaagatgcctggagagac | |
| | | agtcaagatctcctgcaaggcttct | |
| | | ggatataccttcacaaactttggaa | |
| | | tgaactgggtgaagcaggctccagg | |
| | | aaaggctttaaagtggatgggctgg | |
| | | ataaacaccaacactggaaagccaa | |
| | | catattttgaagagttcaagggacg | |
| | | gtttgccttctctttggaaacctct | |
| | | gccagcactgcctatttgcagatca | |
| | | ataatctcaaaaatgaggacacggc | |
| | | tacatatttctgtgcaagacgctgg | |
| | | gaccggtccgcctggtttgcttact | |
| | | ggggccaagggactctggtcactgt | |
| | | ctctgca | |
| 8G4 | VL | caggctgttgtgactcaggaatctg | 20 |
| | | cactcaccacatcacctggtgaaac | |
| | | agtcacactcacttgtcgctcaagt | |
| | | actggggctgttacaactagtaact | |
| | | atgccaactgggtccaagaaaaacc | |
| | | agatcatttattcactggtctaata | |
| | | ggtggtaccaacgaccgatctccag | |
| | | gtgttcctgccagattctcaggctc | |
| | | cctgattggagacaaggctgccctc | |
| | | accatcacaggggcacagactgagg | |
| | | atgaggcaatatatttctgtgctct | |
| | | ctggtacagcaaccattgggtgttc | |
| | | ggtggaggaaccaaactgactgtcc | |
| | | ta | |
| 21G6 | VH | gaggtgcagctggaggagtcgggac | 25 |
| | | ctggcctggtgaaaccttctcagtc | |
| | | tctgtccctcacctgcactgtcact | |
| | | ggctactcaatcaccagtgattatg | |
| | | cctggaactggatccggcagtttcc | |
| | | aggaaacaaactggagtggatgggc | |
| | | tacataagctacagtggtagcacta | |
| | | tctacaacccatctctcaaaagtcg | |
| | | aatctctatcactcgagacacatcc | |
| | | aagaaccagttcttcctgcacttga | |
| | | attctgtgactactgaggacacagc | |
| | | cacatattactgtgcaactctggga | |
| | | cttgactactggggccaaggcacca | |
| | | ctctcacagtctcctca21g6 | |
| 21G6 | VL | gattttgtgatgacgcagtctccat | 30 |
| | | cctccctagctgtgtcagttggaga | |
| | | gaaggttactatgagctgcaagtcc | |
| | | agtcagagccttttatatagtagca | |
| | | atcaaaagaactacttggcctggta | |
| | | ccagcagaaaccagggcagtctcct | |
| | | aaactgctgatttactgggcatcca | |
| | | ctaggggatctggggtccctgatcg | |
| | | cttcacaggcagtggatctgggaca | |
| | | gatttcactctcaccatcagcagtg | |
| | | tgaaggctgaagacctggcagttta | |
| | | ttactgtcagcaatatcatagctat | |
| | | cggacgttcggtggaggcaccaagc | |
| | | tggaaatcaag | |
| M6I-02-A03 | VH | gaagtgcagctgttggagactggag | |
| | | gaggcttggtgcgacctggggggtc | |
| | | acggggactctcttgtgaaggctca | |
| | | gggtttacttttagtggcttctgga | |
| | | tgagctgggttcgacagacacctgg | |
| | | gaagaccctggagtggattggagac | |
| | | attaattctgatggcagtgcagtaa | |
| | | actacgcaccatccataaaggatcg | |
| | | attcactatcttcagagacaatgac | |
| | | aagagcaccctgtacctgcagatga | |
| | | gcaatgtgcgatctgaggacacagc | |
| | | cacgtatttctgtatgagggggact | |
| | | gggtggtacttcgatgtctggggcg | |
| | | cagggaccacggtcaccgtctcctc | |
| | | gg | 35 |
| | VL | gacattgtgatgacacagtctccat | 40 |
| | | cctccctggctgtgtcagcaggaga | |
| | | gaaggtcactatgagctgcaaatcc | |
| | | agtcagagtctgctcaacagtagaa | |
| | | cccgaaagaactacttggcttggta | |
| | | ccagcagaaaccagggcagtctcct | |
| | | aaactgctgatctactgggcatcca | |
| | | ctagggaatctggggtccctgatcg | |
| | | cttcacaggcagtggatctgggaca | |
| | | gatttcactctcaccatcagcagtg | |
| | | tgcaggctgaagacctggcagttta | |
| | | ttactgcaagcaatcttataatctg | |
| | | tacacgttcggaggggggaccaagc | |
| | | tggaaatcaaac | |
| M6I-02-C02 | VH | gacgtgatgctggtggagtctggag | 45 |
| | | gaggcttggtacagcctggggcttc | |
| | | tctgagactctcctgtgcaactttg | |
| | | gggttcaccttcactgactactaca | |
| | | tgagctgggtccgccagcctccagg | |
| | | aaaggcacttgagtggttgggtttt | |
| | | attagagacaaagctaatggttaca | |
| | | caacagagtacagtgcatctgtgaa | |
| | | gggtcggttcaccatctccagagat | |
| | | aattcccaaagcatcctctatcttc | |
| | | aaatgaacaccctgagagctgagga | |
| | | cagtgccacttattactgtgcaaga | |
| | | gatctctttggcctgtactactttg | |
| | | actactggggccagggcaccactct | |
| | | cacagtctcctcgg | |
| | VL | gacattgttctcacccagtctccag | 50 |
| | | caatcatgtctgcatctccagggga | |
| | | gaaggtcaccatgacctgcagtgcc | |
| | | agctcaagtgtaagttacatgcact | |
| | | ggtaccagcagaagtcaggcacctc | |
| | | ccccaaaagatggatttatgacaca | |
| | | tccaaactggcttctggagtccctg | |
| | | ctcgcttcagtggcagtgggtctgg | |
| | | gacctcttattctctcacaatcagc | |
| | | agcatggaggctgaagatgctgcca | |
| | | cttatttctgccatcagtggactac | |
| | | ttacccatacacgttcggagggggg | |
| | | accaagctggaaataaaac | |
| M6I-02-C06 | VH | gacgtgcagctggtggagtctgggg | 55 |
| | | gaggcttagtgaagcctggagggtc | |
| | | cctgaaactctcctgtgcagcctct | |
| | | ggattcactttcagtaactatgcca | |
| | | tgtcttgggttcgccagactccaga | |
| | | gaagaggctggagtgggtcgcatcc | |
| | | attagtagtcgtggttacacctact | |
| | | atgcagacagtgtgaaggcagacag | |
| | | tgtgaagggccgattcaccatctcc | |
| | | agagataatgccaggaacatcctgt | |
| | | acctgcaaatgagcagtctgaggtc | |
| | | tgaggacacggccatgttttactgt | |
| | | gcgagagaactacggctacggtact | |
| | | tcgatgtctggggcgcagggaccac | |
| | | tgtcaccgtctcctcgg | |
| | VL | gatgttgtgctgacccagactccac | 60 |
| | | tcactttgtcggttaccattggaca | |
| | | accagcctccatctcctgcaagtca | |
| | | agtcagagcctcttagatagtgatg | |
| | | gaaagacatatttgaattggttgtt | |
| | | acagaggccaggccagtctccaaag | |
| | | cgcctaatccatctggtgtctaaac | |
| | | tggactctggagtccctgacaggtt | |
| | | tactggcagtggatcagggacagat | |
| | | ttcacactgaaaatcagcagagtgg | |
| | | aggctgaggatttgggagtttatta | |
| | | ttgctggcaaggtacacattttcct | |
| | | cggacgttcggtggaggcaccaagc | |
| | | tggaaatcaaac | |
| M6I-02-D03 | VH | cagatccagttggtgcagtctggac | 65 |
| | | ctgagctgaagaagcctggagagac | |
| | | agtcaggatctcctgcaaggcttct | |
| | | gggtataccttcacaactgctggaa | |
| | | tgcagtgggtgcaaaagatgccagg | |
| | | aaagggtttgaagtggattggctgg | |
| | | ataaacacccactctggagtgccaa | |
| | | aatatgcagaagacttcaagggacg | |
| | | gtttgccttctctttggaaacctct | |
| | | gccagcactgcatatttacagataa | |
| | | gcaacctcaaaaatgaggacacggc | |
| | | tacgtatttctgtgcgagatggcaa | |
| | | gcttttgactactggggccaaggca | |
| | | ccactgtcaccgtctcctcgg | |
| | VL | gatattgtgatgacccagtctcaaa | 70 |
| | | aattcatgtccacatcagtaggaga | |
| | | cagggtcagtgtcacctgcaaggcc | |
| | | agtcagaatgtgggtactaatgtag | |
| | | cctggtatcaacagaaaccagggca | |
| | | atctcctaaagcactgatttactcg | |
| | | gcatcctaccggtacagtggagtcc | |
| | | ctgatcgcttcacaggcagtggatc | |
| | | tgggacagatttcactctcaccatt | |
| | | agcaatgtgcagtctgaagacttgg | |
| | | cagactatttctgtcagcaatataa | |
| | | cagctatcctctcacgttcggaggg | |
| | | gggaccaagctggaaataaaac | |
| M6I-02-D10 | VH | gacgtgaagctggtggagtctgggg | 75 |
| | | gaggcttagtgcagcctggagggtc | |
| | | ccggaaactctcctgtgcagcctct | |
| | | ggattcgctttcaataattttggaa | |
| | | tatactgggttcgtcaggctccaga | |
| | | gaaggggctggagtgggtcgcatac | |
| | | attagtcgtggtaataccatctact | |
| | | atgcagacacagtgaagggccgatt | |
| | | caccatctccagagacaatcccaag | |
| | | aacaccctgttcctgcaaatgacca | |
| | | gtctaaggtctgaggacacggccat | |
| | | gtattactgtggaagatcgtatggt | |
| | | aactacgtgaactactggggcctag | |
| | | gcaccactctcacagtctcctcgg | |
| | VL | gatattgtgatgacccagactccac | 80 |
| | | tcactttgtcggttaccattggaca | |
| | | accagcctccatctcttgcaagtca | |
| | | agtcagagcctcttagatagtgatg | |
| | | gaaagacatatttgaattggttgtt | |
| | | acagaggccaggccagtctccaaag | |
| | | cgcctaatctatctggtgtctaaac | |
| | | tggactctggagtccctgacaggtt | |
| | | cactggcagtggatcagggacagat | |
| | | ttcacactgaaaatcagcagagtgg | |
| | | aggctgaggatttgggagtttatta | |
| | | ttgctggcaaggtacacattttccg | |
| | | tacacgttcggaggggggaccaagc | |
| | | tggaaataaaac | |
| M6I-02-E01 | VH | gaggtgatgctggtggagtctggag | 85 |
| | | gaggcttggtacagcctgggggttc | |
| | | tctgagactctcctgtgcaacttct | |
| | | gggttcaccttcactgattactaca | |
| | | tgagttgggtccgccagcctccagg | |
| | | aaaggcacttgagtggttgggtttt | |
| | | attagaaacaaacctaatggttaca | |
| | | caacagagtacagtgcatctgtgaa | |
| | | gggtcggttcaccatctccagagat | |
| | | aattcccaaagcatcctctatcttc | |
| | | aaatgaacaccctgagagctgagga | |
| | | cagtgccacttattactgtgcaaga | |
| | | gataactacggcctctactttgact | |
| | | actggggccaaggcaccactctcac | |
| | | agtctcctcgg | |
| | VL | gacattgttctcacccagtctccag | 90 |
| | | caatcatgtctgcatctccagggga | |
| | | gaaggtcaccatgacctgcagtgcc | |
| | | agctcaagtataagttacatgcact | |
| | | ggtaccagcagaagccaggcacctc | |
| | | ccccaaaagatggatttatgacaca | |
| | | tccaaactggcttctggagtccctg | |
| | | ctcgcttcagtggcagtgggtctgg | |
| | | gacctcttattctctcacaatcagc | |
| | | agcatggaggctgaagatgctgcca | |
| | | cttattactgccatcagcggagtag | |
| | | ttacccatggacgttcggtggaggc | |
| | | accaagctggaaatcaaac | |
| M6I-04-A02 | VH | gaggttcggctgcaacagtctggac | 95 |
| | | ctgagctggtgaagcctggggcttc | |
| | | agtgaagatatcctgcaagacttct | |
| | | ggatacacattcactgaatacacca | |
| | | tgcactgggtgaagcagagccatgg | |
| | | aaagagccttgagtggattggaggt | |
| | | attgttcctaacaatggtggtacta | |
| | | actacaaccagaagttcaaggtcaa | |
| | | ggccactttgactgtagacaagtcc | |
| | | tccagcacagcctacatggagctcc | |
| | | gcagcctgacatctgaggattctgc | |
| | | agtctattactgtgcaaggagggat | |
| | | tactacggtagtagccactttgact | |
| | | actggggccaaggcaccactctcac | |
| | | agtctcctcgg | |
| | VL | gacattctgatgacccagtctccat | 100 |
| | | cctccctagctgtgtcagttggaga | |
| | | gaaggtcactatgagctgcaaatcc | |
| | | agtcagagtctgctcaacagtagaa | |
| | | cccgaaagaactacttggcctggta | |
| | | ccagcagaaaccagggcagtctcct | |
| | | aaactgctgatttactgggcatcca | |
| | | ctagggaatctggggtccctgatcg | |
| | | cttcacaggcagtggatctgggaca | |
| | | gatttcactctcaccattagcaatg | |
| | | tgcaggctgaagacctggcagttta | |
| | | ttactgcaagcaatcttataatctg | |
| | | tacacgttcggaggggggaccaagc | |
| | | tggaaatcaaac | |
| M6I-04-F11 | VH | caggtccaactgcagcagcctgggg | 105 |
| | | ctgaactggtgaagcctggggcttc | |
| | | agtgaagttgtcctgcaaggcttct | |
| | | ggctacaccttcaccaactcctata | |
| | | tctactgggtgaagcagaggcctgg | |
| | | acaaggccttgagtggattgggggg | |
| | | attgatcctaacaatgatgatacta | |
| | | acttcaatgaggagttcaagagcaa | |
| | | ggccacactgactgtggacaaatcc | |
| | | tccagcacagcctacatgcaactca | |
| | | gcagcctgacatctgaggactctgc | |
| | | ggtctattactgtacaagattacta | |
| | | cgctaccaggcctggtttgcttact | |
| | | ggggccaggggactctggtcaccgt | |
| | | ctcctcgg | |
| | VL | gacattgtgctgacccaatctccag | 110 |
| | | cttctttggctgtgtctctagggca | |
| | | gagggccaccatatcctgcagagcc | |
| | | agtgaaagtgttgatagttatggca | |
| | | atagttttatgcactggtaccagca | |
| | | gaaaccaggacagccacccaaactc | |
| | | ctcatctatcttgcatccaacctag | |
| | | aatctggggtccctgccaggttcag | |
| | | tggcagtgggtctaggacagacttc | |
| | | accctcaccattgatcctgtggagg | |
| | | ctgatgatgctgcaacctattactg | |
| | | tcagcaaaataatgaggatccgtgg | |
| | | acgttcggtggaggcaccaagctgg | |
| | | aaataaaac | |
| M6I-04-H05 | VH | gaggttcggctgcaacagtctggac | 115 |
| | | ctgagctggtgaagcctggggcttc | |
| | | agtgaagatatcctgcaagacttct | |
| | | ggatacacattcactgaatacacca | |
| | | tgcactgggtgaagcagagccatgg | |
| | | aaagagccttgagtggattggaggt | |
| | | attgttcctaacaatggtggtacta | |
| | | actacaaccagaagttcaaggtcaa | |
| | | ggccactttgactgtagacaagtcc | |
| | | tccagcacagcctacatggagctcc | |
| | | gcagcctgacatctgaggattctgc | |
| | | agtctattactgtgcaaggagggat | |
| | | tactacggtagtagccactttgact | |
| | | actggggccaaggcaccactctcac | |
| | | agtctcctcgg | |
| | VL | gatgttgtgatgacccaaactccac | 120 |
| | | tctccctgcctgtcagtcttggaga | |
| | | tcaagcctccatctcttgcagatct | |
| | | agtcagagccttgtacacagtaatg | |
| | | gaaacacctatttacattggtacct | |
| | | gcagaagccaggccagtctccaaag | |
| | | ctcctgatctacaaagtttccaacc | |
| | | gattttctggggtcccagacaggtt | |
| | | cagtggcagtggatcagggacagat | |
| | | ttcacactcaagatcagcagagtgg | |
| | | aggctgaggatctgggagtttattt | |
| | | ctgctctcaaagtacacatgttccg | |
| | | tggacgttcggtggaggcaccaagc | |
| | | tggaaataaaac | |
| M6I-07-A03 | VH | gaggtgcagctcttagaaagcggag | 125 |
| | | gtggcttggttcagcctggcggtag | |
| | | tctaagactgtcctgtgcagcttct | |
| | | ggctttaccctgagcagctattgga | |
| | | tgcattgggtccgtcaagcaccagg | |
| | | taagggtctggaatgggtgtctgca | |
| | | atctctccggcgggtggcggcacct | |
| | | actatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgccagtcctctg | |
| | | tacccatacggcctggatatttggg | |
| | | gccagggaaccctggtcaccgtctc | |
| | | ctcag | |
| | VL | gatattcagatgacccagagcccat | 130 |
| | | cctccctgtcagcttctgtaggaga | |
| | | ccgcgttaccatcacctgtcgtgcc | |
| | | tcccaggatattagcatctacctga | |
| | | actggtatcagcagaagcctggtaa | |
| | | ggcaccgaagctgctgatctatcat | |
| | | gcctccaacctgcaatctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagaacta | |
| | | cggcactccgtatacctttggaggc | |
| | | ggtaccaaggtggagatcaaag | |
| M6I-07-A05 | VH | gaggtgcagctcttagaaagcggag | 135 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 140 |
| | | cctccctgtcagcttctgtaggaga | |
| | | ccgcgttaccatcacctgtcgtgcc | |
| | | tccgagaacattggcgattacctga | |
| | | gctggtatcagcagaagcctggtaa | |
| | | ggcaccgaagctgctgatctatgcc | |
| | | gcatccagcctccagtctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagaacta | |
| | | tgatttcccgctgacttttggaggc | |
| | | ggtaccaaggtggagatcaaag | |
| M6I-07-B05 | VH | gaggtgcagctcttagaaagcggag | 145 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 150 |
| | | cctccctgtcagcttctgtaggaga | |
| | | ccgcgttaccatcacctgtcgtgcc | |
| | | tcccaggatattagcatctacctga | |
| | | actggtatcagcagaagcctggtaa | |
| | | ggcaccgaagctgctgatctatacc | |
| | | gcctccaacctgcaatctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagtccta | |
| | | tagcactctgacctttggaggcggt | |
| | | accaaggtggagatcaaag | |
| M6I-07-C06 | VH | gaggtgcagctcttagaaagcggag | 155 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 160 |
| | | cctccctgtcagcttctgtaggaga | |
| | | ccgcgttaccatcacctgtcgtgcc | |
| | | agccaacagatttccacttacctga | |
| | | actggtatcagcagaagcctggtaa | |
| | | ggcaccgaagctgctgatctatgcc | |
| | | accagccgtctggaagatggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-07-D05 | VH | gaggtgcagctcttagaaagcggag | 165 |
| | | gtggcttggttcagcctggcggtag | |
| | | tctaagactgtcctgtgcagcttct | |
| | | ggcttcccgtttagccactatgcca | |
| | | tgtcttgggtccgtcaagcaccagg | |
| | | taagggtctggaatgggtgtctggc | |
| | | atctccggcagcggtcacaccactg | |
| | | attatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgctcgcgctcgg | |
| | | tttcccccttactacttagacatct | |
| | | ggggccagggaaccctggtcaccgt | |
| | | ctcctcag | |
| | VL | gatattcagatgacccagagcccat | 170 |
| | | cctccctgtcagcttctgtaggaga | |
| | | ccgcgttaccatcacctgtcgtgcc | |
| | | tcccacagcattggcgcatacctga | |
| | | actggtatcagcagaagcctggtaa | |
| | | ggcaccgaagctgctgatctatgcc | |
| | | gcatccagcctccagtctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagtccta | |
| | | tagcactccgaaatttggaggcggt | |
| | | accaaggtggagatcaaag | |
| M6I-07-E01 | VH | gaggtgcagctcttagaaagcggag | 175 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 180 |
| | | cctccctgtcagcttctgtaggaga | |
| | | ccgcgttaccatcacctgtcgtgcc | |
| | | agccaacagatttccacttacctga | |
| | | actggtatcagcagaagcctggtaa | |
| | | ggcaccgaagctgctgatctatgcc | |
| | | accagccgtctggaagatggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-07-F05 | VH | gaggtgcagctcttagaaagcggag | 185 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 190 |
| | | cctccctgtcagcttctgtaggaga | |
| | | ccgcgttaccatcacctgtcgtgcc | |
| | | agccaacagatttccacttacctga | |
| | | actggtatcagcagaagcctggtaa | |
| | | ggcaccgaagctgctgatctatgcc | |
| | | accagccgtctggaagatggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-07-F06 | VH | gaggtgcagctcttagaaagcggag | 195 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctccggtcgcggcggccacacct | |
| | | attatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgcttcacctcct | |
| | | attcgagatgctggaatggatgttt | |
| | | ggggccagggaaccctggtcaccgt | |
| | | ctcctca | |
| | VL | gatattcagatgacccagagcccat | 200 |
| | | cctccctgtcagcttctgtaggaga | |
| | | ccgcgttaccatcacctgtcgtgcc | |
| | | agccagtccgtgaatcgcctgctga | |
| | | actggtatcagcagaagcctggtaa | |
| | | ggcaccgaagctgctgatctatgcc | |
| | | gcatccagcctccagtctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagtccta | |
| | | tagcatcccgcacacctttggaggc | |
| | | ggtaccaaggtggagatcaaag | |
| M6I-07-G03 | VH | gaggtgcagctcttagaaagcggag | 205 |
| | | gtggcttggttcagcctggcggtag | |
| | | tctaagactgtcctgtgcagcttct | |
| | | ggttttaccttcagcaactatgtca | |
| | | tgggctgggtccgtcaagcaccagg | |
| | | taagggtctggaatgggtgtctact | |
| | | atcaccggcccaggtgggagcacct | |
| | | actatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgcacgcgacctg | |
| | | tacggcttcgacgtgtggggccagg | |
| | | gaaccctggtcaccgtctcctcag | |
| | VL | gatattcagatgacccagagcccat | 210 |
| | | cctccctgtcagcttctgtaggaga | |
| | | ccgcgttaccatcacctgtcgtgcc | |
| | | tcccagagcattggcactcacgtga | |
| | | actggtatcagcagaagcctggtaa | |
| | | ggcaccgaagctgctgatctatgcc | |
| | | gcatccagcctccagtctggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-07-H01 | VH | gaggtgcagctcttagaaagcggag | 215 |
| | | gtggcttggttcagcctggcggtag | |
| | | tctaagactgtcatgtgcagcttct | |
| | | ggcttcaactttgctaactacgcga | |
| | | tgaattgggtccgtcaagcaccagg | |
| | | taagggtctggaatgggtgtctgct | |
| | | gtcagcggcagcggtgattataccc | |
| | | actatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgctagagatttc | |
| | | tggtgggggctggagggaatggact | |
| | | attggggccagggaaccctggtcac | |
| | | cgtctcctcag | |
| | VL | gatattcagatgacccagagcccat | 220 |
| | | cctccctgtcagcttctgtaggaga | |
| | | ccgcgttaccatcacctgtcgtgcc | |
| | | agccagaccattaagaactacgtga | |
| | | attggtatcagcagaagcctggtaa | |
| | | ggcaccgaagctgctgatctatgca | |
| | | gcctccaactttcagagcggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagtccta | |
| | | cagcatcccgtattcttttggaggc | |
| | | ggtaccaaggtggagatcaaag | |
| M6I-07-H04 | VH | gaggtgcagctcttagaaagcggag | 225 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 230 |
| | | cctccctgtcagcttctgtaggaga | |
| | | ccgcgttaccatcacctgtcgtgcc | |
| | | agccaacagatttccacttacctga | |
| | | actggtatcagcagaagcctggtaa | |
| | | ggcaccgaagctgctgatctatgcc | |
| | | accagccgtctggaagatggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-07-H05 | VH | gaggtgcagctcttagaaagcggag | 235 |
| | | gtggcttggttcagcctggcggtag | |
| | | tctaagactgtcctgtgcagcttct | |
| | | ggcttttccctgagcgattatgaca | |
| | | tgacctgggtccgtcaagcaccagg | |
| | | taagggtctggaatgggtgtctact | |
| | | atccgcgcaagtgacgagtccacct | |
| | | actatgccgatagcgtgaaaggacg | |
| | | cttcaccatcagcagggacaattcc | |
| | | aagaacacgctgtacctgcaaatga | |
| | | acagcctgcgagccgaggatacggc | |
| | | cgtgtattactgtgccagcggttcc | |
| | | ggagctctgtacgatcccttcgatg | |
| | | tgtggggccagggaaccctggtcac | |
| | | cgtctcctcag | |
| | VL | gatattcagatgacccagagcccat | 240 |
| | | cctccctgtcagcttctgtaggaga | |
| | | ccgcgttaccatcacctgtcgtgcc | |
| | | agccagaccattaagaactacgtga | |
| | | attggtatcagcagaagcctggtaa | |
| | | ggcaccgaagctgctgatctatgca | |
| | | gcctccaactttcagagcggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagtccta | |
| | | tagcaccccaccgacttttggaggc | |
| | | ggtaccaaagtggagatcaaag | |
| M6I-07-H06 | VH | gaggtgcagctcttagaaagcggag | 245 |
| | | gtggcttggttcagcctggcggtag | |
| | | tctaagactgtcctgtgcagcttct | |
| | | ggcttcacctttagcaactatgcga | |
| | | tgggttgggtccgtcaagcaccagg | |
| | | taagggtctggaatgggtgtctgtc | |
| | | atctcttccggtggcggtggtggaa | |
| | | cctcttatgccgatagcgtgaaagg | |
| | | acgcttcaccatcagcagggacaat | |
| | | tccaagaacacgctgtacctgcaaa | |
| | | tgaacagcctgcgagccgaggatac | |
| | | ggccgtgtattactgtgcttcacct | |
| | | cctcgagactacgagggcatggatg | |
| | | tttggggccagggaaccctggtcac | |
| | | cgtctcctcag | |
| | VL | gatattcagatgacccagagcccat | 250 |
| | | cctccctgtcagcttctgtaggaga | |
| | | ccgcgttaccatcacctgtcgtgcc | |
| | | agccaacagatttccacttacctga | |
| | | actggtatcagcagaagcctggtaa | |
| | | ggcaccgaagctgctgatctatgcc | |
| | | accagcaacctgcacggtggcgtcc | |
| | | cgtcacgctttagtggctctggttc | |
| | | tgggaccgacttcactctcaccatt | |
| | | agcagtctgcaaccggaggacttcg | |
| | | cgacatactattgccagcagacata | |
| | | tatcaccccgcgcacctttggaggc | |
| | | ggtaccaaggtggagatcaaag | |
| M6I-08-A05 | VH | caggttcaacttcaggaaagtggtc | 255 |
| | | ctgggcttgtcaagccgtctcagac | |
| | | gctctcgctaacctgtactgtgtca | |
| | | gggtactccattacaagtgactatg | |
| | | cctggaattggattcggcagccacc | |
| | | cggtaaaggactggagtggattggg | |
| | | tatatctcctatagcggcagcacta | |
| | | tatacaatcccagcctgaagtccag | |
| | | gatctcaatcagcgtcgacacctct | |
| | | aaaaaccagttctcactgaagctca | |
| | | actccgtaaccgctgccgatacagc | |
| | | cgtgtactactgcgcaactttaggc | |
| | | ctggactattggggacaaggcacct | |
| | | tggttacagtgtcctcag | |
| | VL | gatattgtgatgactcaaagccctg | 260 |
| | | actctctagccgtgtccctcggtga | |
| | | gcgggcaacgataaactgcaagtcc | |
| | | tctcagtccctgctctatagcagca | |
| | | accagaagaattacctggcttggta | |
| | | tcaacagaagcccggacagccgccc | |
| | | aaattgttaatctattgggcttcga | |
| | | cacgcggttcaggcgtaccagacag | |
| | | atttagcgggagtgggtctggcaca | |
| | | gatttcacccttactatctccagtc | |
| | | tgcaggccgaggacgtggccgttta | |
| | | ctactgccagcaataccactcatat | |
| | | aggaccttcggccagggaaccaaag | |
| | | tcgaaattaaag | |
| M6I-08-B05 | VH | gaagtgaagctggtggagtctgggg | 265 |
| | | gaggcttagtgaagcctggagggtc | |
| | | cctgaaactctcctgtgcagcctct | |
| | | ggattcactttcagtacctatgcca | |
| | | tgtcttgggttcgccagactccgga | |
| | | gaagaggctggagtgggtcgcaacc | |
| | | attagtgatggtggtcgttacacct | |
| | | actatccagacagtgtgaaggggcg | |
| | | attcaccatctccagagacaatgcc | |
| | | aagaacaacctgtacctgcaaatga | |
| | | gcagtctaaagtctgaggacacagc | |
| | | catgtattactgtgcaagacgggac | |
| | | ggtttcttctttgactcctggggcc | |
| | | aaggcaccactctcacagtctcctc | |
| | | gg | |
| | VL | gacacaactgtgacccagtctcaaa | 270 |
| | | aattcatgtccacatcagtaggaga | |
| | | cagggtcagcatcacctgcaaggcc | |
| | | agtcagaatgtgggtactgctgtag | |
| | | cctggtatcaacagaaaccaggaca | |
| | | atctcctaaactactgatttactcg | |
| | | gcatccaatcggtacactggagtcc | |
| | | ctgatcgcttcacaggcagtggatc | |
| | | tgggacagatttcactctcaccatc | |
| | | agcagtgtgcaggctgaagacctgg | |
| | | cagtttattactgcaagcaatctta | |
| | | taatctgtacacgttcggagggggg | |
| | | accaagctggaaatgaaac | |
| M6I-09-A08 | VH | caggtgcaactgcagcagcctgggg | 275 |
| | | ctgaactggtgaagcctggggcttc | |
| | | agtgaagctgtcctgcaaggcttct | |
| | | ggctacaccttcaccagctactgga | |
| | | tgcactgggtgaagcagaggcctgg | |
| | | acaaggccttgagtggattggagag | |
| | | attaatcctagcaacggtcgtacta | |
| | | actacaatgagaagttcaagagcaa | |
| | | ggccacactgactgtagacaaatcc | |
| | | tccagcacagcctacatgcagctca | |
| | | gcagcctgacatctgaggactctgc | |
| | | ggtctattactgtgcaaactgggac | |
| | | tactggggccaaggcaccactctca | |
| | | cagtctcctcg | |
| | VL | gatgttgtgatgacccagtctcaaa | 280 |
| | | aattcttgtccacatcagtaggaga | |
| | | cagggtcggcgtcacctgcaaggcc | |
| | | agtcagaatgtgggtactaatgtag | |
| | | cctggtatcaacagaaaccagggca | |
| | | atctcctaaactactgatttactgg | |
| | | gcatccacccggcacactggagtcc | |
| | | ctgatcgcttcacaggcagtggatc | |
| | | tgggacagatttcactctcaccatt | |
| | | agcaatgtgcagtctgaagacttgg | |
| | | cagattatttctgtcagcaatatag | |
| | | cagctatcctctgacgttcggtgga | |
| | | ggcaccaagctggaaatgaaac | |
| M6I-09-B03 | VH | caggtgcaactgaaggagtctgggg | 285 |
| | | ctgaactggtgaagcctggggcttc | |
| | | agtgaaattgtcctgcaaggcttct | |
| | | ggctacaccttcaccaactcctata | |
| | | tctactgggtgaagcagaggcctgg | |
| | | acaaggccttgagtggattgggggg | |
| | | attgatcctaacaatgatgatacta | |
| | | acttcaatgaggagttcaagagcaa | |
| | | ggccacactgactgtggacaaatcc | |
| | | tccagcacagcctacatgcaactca | |
| | | gcagcctgacatctgaggactctgc | |
| | | ggtctattactgtacaagattacta | |
| | | cgctaccaggcctggtttgcttact | |
| | | ggggccaggggactctggtcaccgt | |
| | | ctcctcgg | |
| | VL | gatgttgtgatgacccagtctcaaa | 290 |
| | | aattcatgtccacatcagtaggaga | |
| | | cagggtcagcgtcacctgcaaggcc | |
| | | agtcagaatgtgggtactaatgtag | |
| | | cctggtatcaacagaaaccagggca | |
| | | atctcctaaagcactgatttactcg | |
| | | gcatcctaccggtacagtggagtcc | |
| | | ctgatcgcttcacaggcagtggatc | |
| | | tgggacagatttcactctcaccatc | |
| | | agcaatgtgcagtctgaagacttgg | |
| | | cagagtatttctgtcagcaatataa | |
| | | cagctatccgtacacgttcggaggg | |
| | | gggaccaagctggaaatcaaac | |
| M6I-09-D05 | VH | caggtccaactgcagcagcctgggg | 295 |
| | | ctgaactggtgaagcctggggcttc | |
| | | agtgaaattgtcctgcaaggcttct | |
| | | ggctacaccttcaccaactactata | |
| | | tatactgggtgaaacagaggcctgg | |
| | | acaaggccttgagtggattggagag | |
| | | attaatcctagcaatggtggtactc | |
| | | acttcaatgagaagttcgagagcaa | |
| | | ggccacactgactgtagacaagtcc | |
| | | tccagcacaacatacatgcaactca | |
| | | acagcctgacatctgaggactctgc | |
| | | ggtctattactgtgtctatgatggt | |
| | | tactatagggataggtatttcgatg | |
| | | tctggggcgcagggaccacggtcac | |
| | | cgtctcctcgg | |
| | VL | gacattgtgctgacccaatctccag | 300 |
| | | cttctttggctgtgtctctagggca | |
| | | gagggccaccatatcctgcagagcc | |
| | | agtgaaagtgttgatagttatggca | |
| | | atagttttatgcactggtaccagca | |
| | | gaaaccaggacagccacccaaactc | |
| | | ctcatctatcttgcatccaacctag | |
| | | aatctggggtccctgccaggttcag | |
| | | tggcagtgggtctaggacagacttc | |
| | | accctcaccattgatcctgtggagg | |
| | | ctgatgatgctgcaacctattactg | |
| | | tcagcaaaataatgaggatccgtgg | |
| | | acgttcggtggaggcaccaagctgg | |
| | | aaataaaac | |
| M6I-09-F08 | VH | gaggtgaagctggtggagtctgggg | 305 |
| | | gaggcttagtgcagcctggagggtc | |
| | | ccggaaactctcctgtgcagcctct | |
| | | ggattcactttcagtacctatgcca | |
| | | tgtcttgggttcgccagactccgga | |
| | | gaagaggctggagtgggtcgcaacc | |
| | | attagtgatggtggtcgttacacct | |
| | | actatccagacagtgtgaaggggcg | |
| | | attcaccatctccagagacaatgcc | |
| | | aagaacaacctgtacctgcaaatga | |
| | | gcagtctaaagtctgaggacacagc | |
| | | catgtattactgtgcaagacgggac | |
| | | ggtttcttctttgactcctggggcc | |
| | | aaggcaccactctcaccgtctcctc | |
| | | gg | |
| | VL | gatattgtgatgacccagtctcaaa | 310 |
| | | aattcatgtccacatcagtaggaga | |
| | | cagggtcagcgtcacctgcaaggcc | |
| | | agtcagaatgtgggtactaatgtag | |
| | | cctggtatcaacagaaaccagggca | |
| | | atctcctaaagcactgatttactcg | |
| | | gcatcctaccggtacagtggagtcc | |
| | | ctgatcgcttcacaggcagtggatc | |
| | | tgggacagatttcactctcacaatc | |
| | | agcagcatggaggctgaagatgctg | |
| | | ccatttattactgccagcagtggag | |
| | | tagtaacccacccatcacgttcgga | |
| | | ggggggaccaagctggaaatgaaac | |
| M6I-10-D03 | VH | caggtccaactgcagcagtctggac | 315 |
| | | ctgaactgaagaagcctggagagac | |
| | | agtcaggatctcctgcaaggcttct | |
| | | ggctataccttcacagactattcaa | |
| | | tatactgggtgaagcaggctccagg | |
| | | acagggtttaaagttgatgggctgg | |
| | | ataaacactgcgactggtgattcaa | |
| | | catatgcggatgacttccagggacg | |
| | | gtttgccttctctttggaaacctct | |
| | | gccagcactgcctatttgcagatca | |
| | | acaacctcaaaaatgacgacacggc | |
| | | tacatatttctgtgctaggacgacc | |
| | | ttctggggccaaggcaccactctca | |
| | | cagtctcctcgg | |
| | VL | gatgttgtgatgacccagtctcaca | 320 |
| | | aattcatgtccacatcagtaggaga | |
| | | cagggtcagcgtcacctgcaaggcc | |
| | | agtcagaatgtgggtactaatgtag | |
| | | cctggtatcaacagaaaccagggca | |
| | | atctcctaaagcactgatttactcg | |
| | | gcatcctaccggtacagtggagtcc | |
| | | ctgatcgcttcacaggcagtggatc | |
| | | tgggacagatttcactctcaccatc | |
| | | agcaatgtgcagtctgaagacttgg | |
| | | cagactatttctgtcagcaatataa | |
| | | cagctatcctctgacgttcggtgga | |
| | | ggcaccaagctggaaatgaaac | |
| M6I-10-D10 | VH | caggtccaactgcagcagcctgggg | 325 |
| | | ctgaactggtgaagcctggggcttc | |
| | | agtgaagttgtcctgcaaggcttct | |
| | | ggctacaccttcaccaactcctata | |
| | | tctactgggtgaagcagaggcctgg | |
| | | acaaggccttgagtggattgggggg | |
| | | attgatcctaacaatgatgatacta | |
| | | acttcaatgaggagttcaagagcaa | |
| | | ggccacactgactgtggacaaatcc | |
| | | tccagcacagcctacatgcaactca | |
| | | gcagcctgacatctgaggactctgc | |
| | | ggtctattactgtacaagattacta | |
| | | cgctaccaggcctggtttgcttact | |
| | | ggggccaggggactctggtcaccgt | |
| | | ctcctcgg | |
| | VL | gacattgtgctgacccaatctccag | 330 |
| | | cttctttggctgtgtctctagggca | |
| | | gagggccaccatatcstgcagagcc | |
| | | agtgaaagtgttgatagttatggca | |
| | | atagttttatgcactggtaccagca | |
| | | gaaaccaggacagccacccaaactc | |
| | | ctcatctatcttgcatccaacctag | |
| | | aatctggggtccctgccaggttcag | |
| | | tggcagtgggtctaggacagacttc | |
| | | accctcaccattgatcctgtggagg | |
| | | ctgatgatgctgcaacctattactg | |
| | | tcagcaaaataatgaggatccgtgg | |
| | | acgttcggtggaggcaccaagctgg | |
| | | aaataaaac | |
| M6I-11-D08 | VH | gaggtgaagctggtggagtctgggg | 335 |
| | | gaggcttagtgaagcctggagggtc | |
| | | cctgaaactctcctgtgcagcctct | |
| | | ggattcactttcagtagctttggaa | |
| | | tgcactgggttcgtcaggctccaga | |
| | | gaaggggctggagtgggtcgcatac | |
| | | attagtagtggtggtaattacacct | |
| | | actattcagacagtgtgaagggtcg | |
| | | attcaccgtatccagagacaatgcc | |
| | | aagaacaccctgttcctgcaaatga | |
| | | gcagtctgaggtctgaggacacggc | |
| | | catgtattactgtgcaagagatgat | |
| | | aggtacgacgactttgactactggg | |
| | | gccaaggcaccactctcaccgtctc | |
| | | ctcgg | |
| | VL | gacacaactgtgacccagtctccag | 340 |
| | | caatcatgtctgcatctccagggga | |
| | | gaaggtcaccatgacctgcagtgcc | |
| | | agctcaagtgtaagttacatgtact | |
| | | ggtaccagcagaagccaggatcctc | |
| | | ccccagactcctgatttatgacaca | |
| | | tccaacctggcttctggagtccctg | |
| | | ctcgcttcagtggcagtgggtctgg | |
| | | gacctcttactctctcacaatcagc | |
| | | agcatggaggctgaagatgctgcca | |
| | | cttattactgccagcaatatagcag | |
| | | ctatccgtacacgttcggagggggg | |
| | | accaagctggaaatcaaac | |
| M6I-11-D10 | VH | gaggtgcagctagttgagtctgggg | 345 |
| | | gaggcttagtgcagcctggagggtc | |
| | | ccggaaactctcctgtgcagcctct | |
| | | ggattcgctttcagtacctttggaa | |
| | | tgcactggattcgtcaggctccaga | |
| | | gaaggggctggagtgggtcgcatcc | |
| | | attagtagtggtggtaattacacct | |
| | | actattcagacagtgtgaagggtcg | |
| | | attcaccgtatccagagacaatgcc | |
| | | aagaacaccctgttcctgcaaatga | |
| | | gcagtctgaggtctgaggacacggc | |
| | | catgtattactgtgcaagagatgat | |
| | | aggtacgacgactttgactactggg | |
| | | gccaaggcaccactctcaccgtctc | |
| | | ctcgg | |
| | VL | gacacaactgtgacccagtctccag | 350 |
| | | caatcatgtctgcatctccagggga | |
| | | gaaggtcaccatgacctgcagtgcc | |
| | | agctcaagtgtaagttacatgtact | |
| | | ggtaccagcagaagccaggatcctc | |
| | | ccccagactcctgatttatgacaca | |
| | | tccaacctggcttctggagtccctg | |
| | | ctcgcttcagtggcagtgggtctgg | |
| | | gacctcttactctctcacaatcagc | |
| | | agcatggaggctgaagatgctgcca | |
| | | cttattactgccagcagtggagtag | |
| | | taacccacccacgttcggagggggg | |
| | | accaagctggaaatcaaac | |
| M6I02-G04 | VH | gaagtgaagctggtggagtctggag | 355 |
| | | gaggcttggtacagcctgggggttc | |
| | | tctgagactctcctgtacaacttct | |
| | | gggttcaccttcactgattactaca | |
| | | tgagctgggtccgccagcctccagg | |
| | | aaaggcacttgagtggttgggtttt | |
| | | attagaaacaaagctaatggttaca | |
| | | caacagagtacagtacatctgtgaa | |
| | | gggtcggttcaccatctccagagat | |
| | | aattcccacagcatcctctatcttc | |
| | | aaatgaacaccctgagagctgagga | |
| | | cagtgccacttattactgtgcaaga | |
| | | gattactacggtagtttgtttgctt | |
| | | actggggccaagggactctggtcac | |
| | | tgtctctgcgg | |
| | VL | gacaatgttctcacccagtctccag | 360 |
| | | cactcatgtctgcatctccagggga | |
| | | gaaggtcaccatgacctgcagtgcc | |
| | | agctcaagtgtaagttacatgcact | |
| | | ggtaccagcagaagtcaggcacctc | |
| | | ccccaaaagatggatttatgacaca | |
| | | tccaaactggcttctggagtccctg | |
| | | ctcgcttcagtggcagtgggtctgg | |
| | | gacctcttactctctcacaatcagc | |
| | | agcatggaggctgaagatgctgcca | |
| | | cttattactgccagcagtggagtag | |
| | | taacccacccacgttcggtggaggc | |
| | | accaagctggaaataaaa | |
| Hum1D7 | VH | gaagtgcaactggtggagtcggggg | 362 |
| | | gggcttggtaaagcctgggggctct | |
| | | ctcagactgagctgtgttgcatcag | |
| | | gattcacattttcttccttcgctat | |
| | | gtcttgggttcggcaggcacccggc | |
| | | aaaggactggaatgggtcgccatta | |
| | | tcagttccggaggcggctatatcta | |
| | | ttacgccgacagcatgactgggcgg | |
| | | ttcaccatatcaagggacaatgcta | |
| | | agaacagtctctacttacagatgtc | |
| | | ctccctacgcgcagaggatacggcc | |
| | | gtctactactgcgcccgacttgggg | |
| | | gtgattacgacgagttttatgccaa | |
| | | agattattggggtcagggaaccctg | |
| | | gtgacagtgagcagc | |
| | VH | gaagtgcaactggtggaatctggtg | 364 |
| | | ggggacttgtaaaacctggcggttc | |
| | | actgaggctctcctgtgccgcctca | |
| | | ggcttcaccttttccagtttcgcta | |
| | | tgagttgggtgcgtcaggcacccgg | |
| | | caaggggttagagtgggtgtctatt | |
| | | atcagcagcggaggcggctatatat | |
| | | actatgctgattccatgacagggag | |
| | | attcacgatctcgcgggacaacgcc | |
| | | aagaacagcttgtacctacagatga | |
| | | atagcctgcgagccgaggacacagc | |
| | | agtctattactgcgcacgcctcgga | |
| | | ggtgattacgatgagttttacgcca | |
| | | aagactattggggacaggggaccct | |
| | | ggtcactgtttcttcc | |
| Hum1D7 | VH | gaggtacaacttgtggaatcaggag | 366 |
| | | gggggctagtccagcctggaggaag | |
| | | tctccgattgtcttgtgctgcctcc | |
| | | ggattcactttttcttcatttgcca | |
| | | tgtcctgggtcagacaggctcccgg | |
| | | taagggcttagagtgggtgagcata | |
| | | atcagcagcgggggcggctatatct | |
| | | attatgcagatagcatgacggggcg | |
| | | gttcaccatttcccgcgacaacagt | |
| | | aagaataccctgtacctccagatga | |
| | | actcgctgagggcagaagacacagc | |
| | | agtttactactgcgccaaactgggc | |
| | | ggtgattatgacgagttctacgcca | |
| | | aagattactggggtcagggcacaat | |
| | | ggtgactgtgtcctct | |
| | VL | gacatccagatgactcagtctcctt | 368 |
| | | caagtctgagcgcttcggtgggcga | |
| | | ccgcgtcacaattacatgcaaagcc | |
| | | agtgaagacatctacaaccggctcg | |
| | | cctggtatcaacagaagcccggcaa | |
| | | agcaccaagactgctgatatatgga | |
| | | gctacgtccttagagacaggtgtac | |
| | | cgtccaggtttagcgggagcgggtc | |
| | | cggcactgatttcactttgaccatt | |
| | | acctcacttcagcccgaagacgttg | |
| | | ccacctactattgtcagcaatactg | |
| | | gtctacctggacattcggccaggga | |
| | | accaaggtggagatcaag | |
| Hum1D7 | VL | gacattcagatgacacagtctccta | 370 |
| | | gctcgcttagtgcttccgtgggaga | |
| | | tcgcgtgaccatcacatgtaaagcc | |
| | | tcagaagacatctacaacagactgg | |
| | | cctggtatcagcagaagccagggaa | |
| | | agcacccaagctcctgatatacggt | |
| | | gccacatctttagagacaggcgttc | |
| | | cgtccaggttcagcgggagcggttc | |
| | | tggaactgactttactttgaccatc | |
| | | tccagtctgcaacccgaggatgtcg | |
| | | ctacctactattgccaacagtattg | |
| | | gtcaacttggacgttcggccagggc | |
| | | accaaagtggaaattaag | |
| | VL | gatatacagatgacacagtctcctt | 372 |
| | | cttccctgtcggcttcagtgggtga | |
| | | tcgggttactattacctgcaaggcc | |
| | | agcgaggacatctataacaggctgg | |
| | | cctggtatcagcagaaacccggaaa | |
| | | ggctccgaagctgttaatctatggt | |
| | | gcaacatccctcgaaacaggcgtcc | |
| | | catccagattcagtggctcagggag | |
| | | cggaacagactttacccttacgatc | |
| | | tccagtttgcagcccgaggactttg | |
| | | ccacctactactgtcagcaatactg | |
| | | gagcacttggaccttcggccaaggc | |
| | | actaaagtggaaattaaa | |

In some embodiments, provided herein is an anti-MUC16 antibody, or binding fragment thereof, wherein the antibody comprises the amino acid sequences of the following three VH CDRs: SEQ ID NOS: 2, 3, 4; 12, 13, 14; 22, 23, 24; 32, 33, 34; 42, 43, 44; 52, 53, 54; 62, 63, 64; 72, 73, 74; 82, 83, 84; 92, 93, 94; 102, 103, 104; 112, 113, 114; 122, 123, 124; 132, 133, 134; 142, 143, 144; 152, 153, 154; 162, 163, 164; 172, 173, 174; 182, 183, 184; 192, 193, 194; 202, 203, 204; 212, 213, 214; 222, 223, 224; 232, 233, 234; 242, 243, 244; 252, 253, 254; 262, 263, 264; 272, 273, 274; 282, 283, 284; 292, 293, 294; 302, 303, 304; 312, 313, 314; 322, 323, 324; 332, 333, 334; 342, 343, 344; and 352, 353, 354; and comprises the amino acid sequences of the following three VL CDRs SEQ ID NOS: 7, 8, 9; 17, 18, 19; 27, 28, 29; 37, 38, 39; 47, 48, 49; 57, 58, 59; 67, 68, 69; 77, 78, 79; 87, 88, 89; 97, 98, 99; 107, 108, 109; 117, 118, 119; 127, 128, 129; 137, 138, 139; 147, 148, 149; 157, 158, 159; 167, 168, 169; 177, 178, 179; 187, 188, 189; 197, 198, 199; 207, 208, 209; 217, 218, 219; 227, 228, 229; 237, 238, 239; 247, 248, 249; 257, 258, 259; 267, 268, 269; 277, 278, 279; 287, 288, 289; 297, 298, 299; 307, 308, 309; 317, 318, 319; 327, 328, 329; 337, 338, 339; 347, 348, 349; and 357, 358, 359.

In some embodiments, provided herein is an anti-MUC16 antibody, or binding fragment thereof, a VH comprising the amino acid sequence having at least 96, 97, 98, 99, or 100% sequence identity with any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and a VL comprising the amino acid sequence having at least 96, 97, 98, 99, or 100% sequence identity with any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.

In some embodiments, provided herein is an anti-MUC16 antibody, or binding fragment thereof, wherein the VH is a nucleic acid having at least 96, 97, 98, 99, or 100% sequence identity with SEQ ID NO: 5; 15; 25; 35; 45; 55; 65; 75; 85; 95; 105; 115; 125; 135; 145; 155; 165; 175; 185; 195; 205; 215; 225; 235; 245; 255; 265; 275; 285; 295; 305; 315; 325; 335; 345; or 355, and the VL is a nucleic acid having at least 95, 96, 97, 98, 99, or 100% sequence identity with SEQ ID NO: 10; 20; 30; 40; 50; 60; 70; 80; 90; 100; 110; 120; 130; 140; 150; 160; 170; 180; 190; 200; 210; 220; 230; 240; 250; 260; 270; 280; 290; 300; 310; 320; 330; 340; 350, or a humanized version thereof.

The term variable domain and variable region are used interchangeably and refer to the portions of the light and heavy chains of an antibody that include the complementarity determining regions and framework regions (FRs).

Table 4 provides amino and nucleic acid sequences for the variable domains of exemplary humanized anti-MUC16 antibodies of the disclosure. Accordingly, in some embodiments anti-MUC16 antibody of the disclosure comprises variable heavy chains comprising an amino acid sequence and variable light chain set for therein, and combination thereof.

Exemplary humanized variable heavy chain amino acid sequences using 1D7 Anti-MUC16 Clone as an example are HC SEQ ID NOS: 361, 363, and 365.

Exemplary humanized variable heavy chain nucleic acid sequences using 1D7 Anti-MUC16 Clone as an example are HC SEQ ID NOS: 362, 364, and 366.

Exemplary humanized variable light chain amino acid sequences using 1D7 Anti-MUC16 Clone as an example are HC SEQ ID NOS: 367, 369, and 371.

Exemplary humanized variable light chain nucleic acid sequences using 1D7 Anti-MUC16 Clone as an example are HC SEQ ID NOS: 368, 370, and 372.

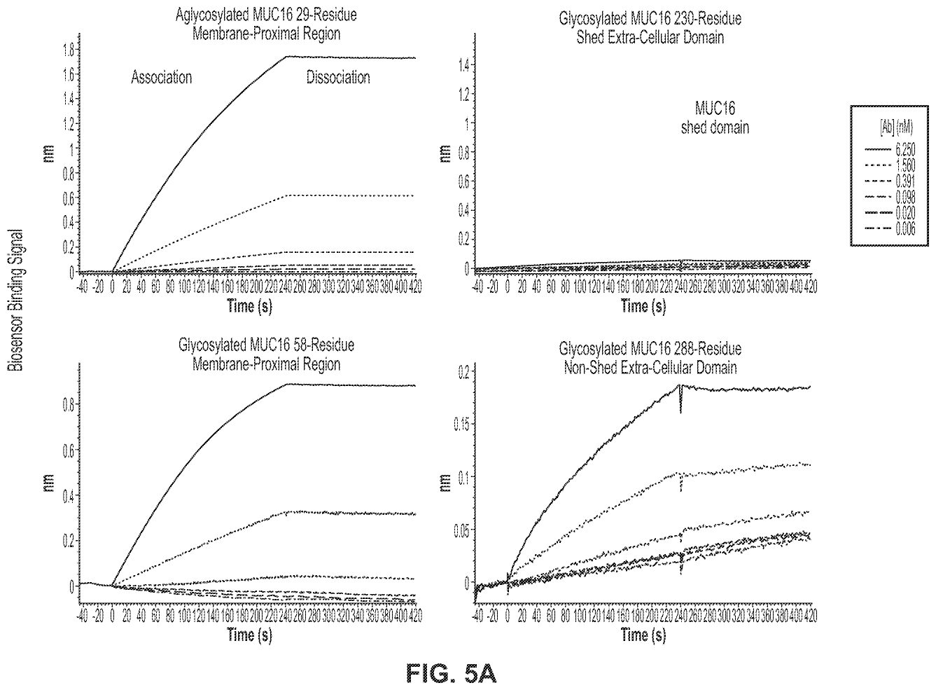

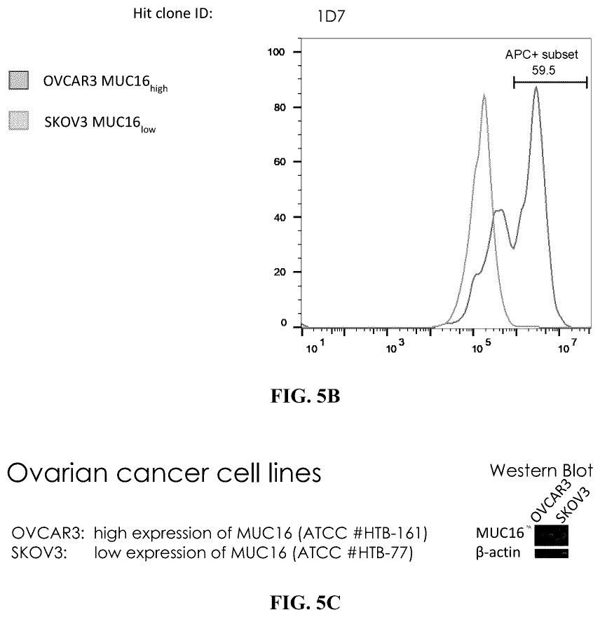

FIG. 5A are graphs that show that the 1D7 anti-MUC16 clone binds a membrane-proximal aglycosylated epitope and does not bind the MUC16 shed domain.

Methods: Biotinylated 29-mer engineered epitope, glycosylated 58-mer engineered epitope, 230-mer MUC16 shed domain and 288-mer MUC16 extra-cellular domain were immobilized on a Pall Octet 384-Red streptavidin biosensor. Immobilized biosensor tips were dipped into increasing concentration of purified antibody with a 4 minute association followed by a 3 minute dissociation.

FIG. 5B is a graph that shows that the 1D7 anti-MUC16 clone specifically binds MUC16_{high} vs. MUC16_{low} cells.

Methods: Hybridoma supernatants were diluted 1:2 into FACS buffer containing OVCAR-3 or SKOV-3 cells and incubated for 30 minutes, then washed once with FACS buffer. 2 ug/mL APC labeled goat anti-mouse Fc secondary antibody diluted in FACS buffer was added to the cells and incubated for 20 minutes, then washed once with FACS buffer. Flow cytometry was performed with 20,000 cells per sample.

FIG. 5C is a western blot that shows the differential expression of MUC16 on OVCAR3 ovarian cancer cells and SKOV3 ovarian cancer cells to corroborate the differential cell binding observed in FIG. 5B.

FIG. 5C reference: Zhai Y, Lu Q, Lou T, Cao G, Wang S, Zhang Z. MUC16 affects the biological functions of ovarian cancer cells and induces an antitumor immune response by activating dendritic cells. Ann Transl Med. 2020 November; 8(22):1494. doi: 10.21037/atm-20-6388. PMID: 33313239; PMCID: PMC7729312.

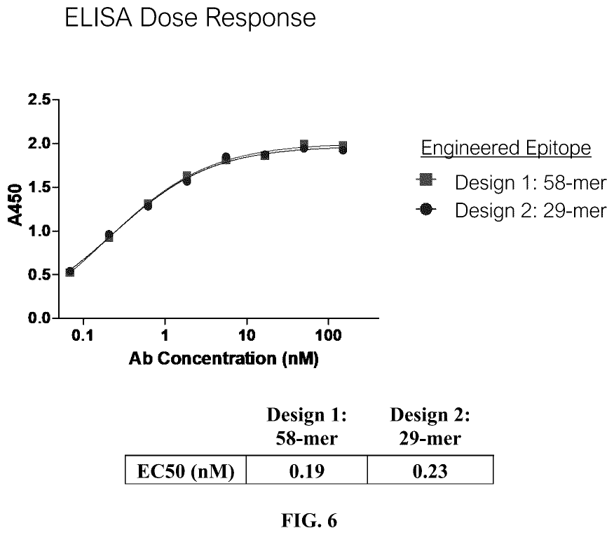

FIG. 6 is a graph that shows an ELISA dose-response curve of clone 8G4 binding to both the engineered epitope 1 and engineered epitope 2 peptides.

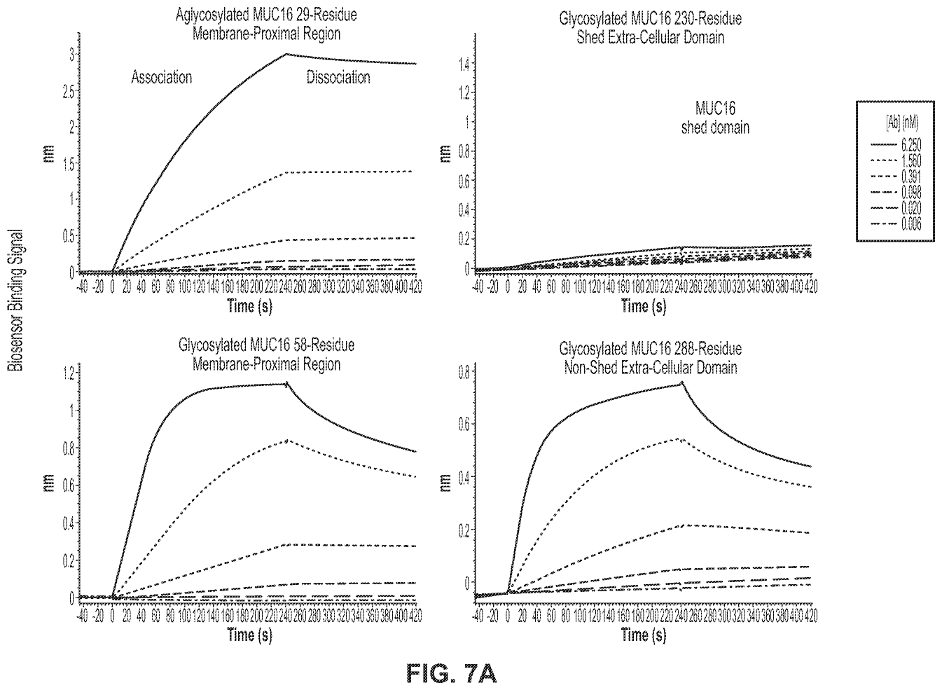

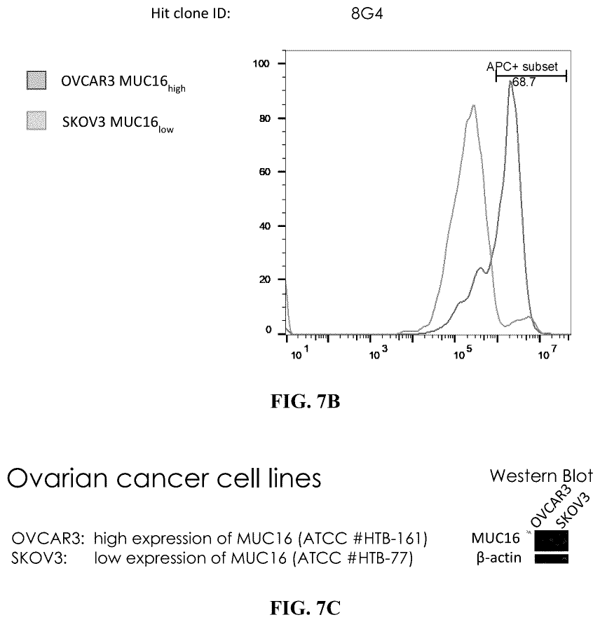

FIG. 7A are graphs that show that the 8G4 anti-MUC16 clone binds a membrane-proximal aglycosylated epitope and does not bind the MUC16 shed domain.

FIG. 7B is a graph that shows that the 8G4 anti-MUC16 clone specifically binds MUC16_{high} vs. MUC16_{low} cells.

FIG. 7C is a western blot that shows the differential expression of MUC16 on OVCAR3 ovarian cancer cells and SKOV3 ovarian cancer cells to corroborate the differential cell binding observed in FIG. 7B.

FIG. 7C reference: Zhai Y, Lu Q, Lou T, Cao G, Wang S, Zhang Z. MUC16 affects the biological functions of ovarian cancer cells and induces an antitumor immune response by activating dendritic cells. Ann Transl Med. 2020 November; 8(22): 1494. doi: 10.21037/atm-20-6388. PMID: 33313239; PMCID: PMC7729312.

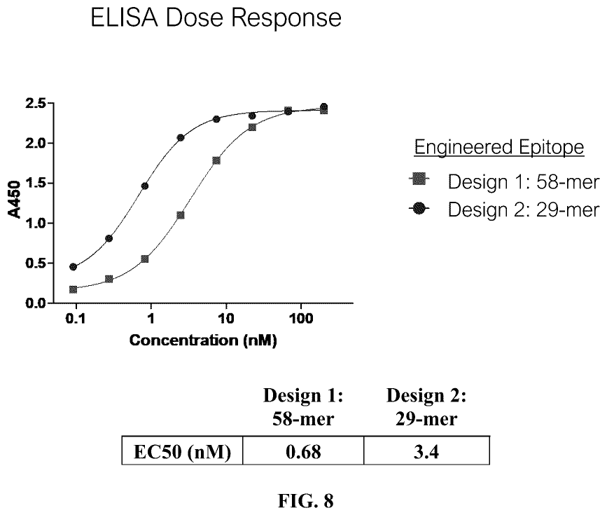

FIG. 8 is a graph that shows an ELISA dose-response curve of clone 21G6 anti-MUC16 clone identified in a hybridoma ELISA screen using the engineered 58-mer antigen—engineered epitope design 1.

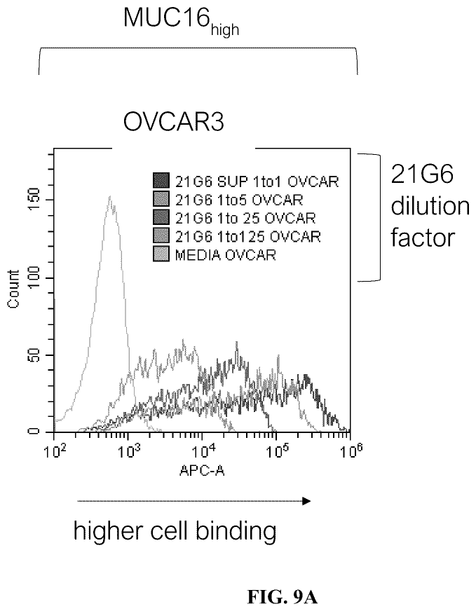

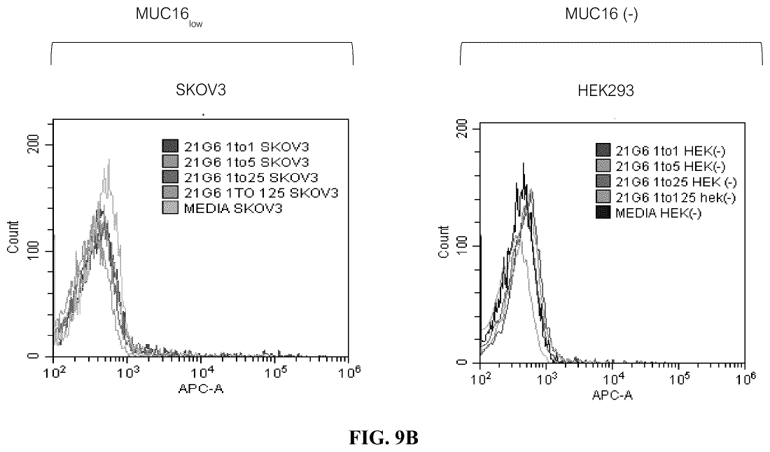

FIG. 9A is a graph that shows 21G6 Anti-MUC16 clone specifically binds OVCAR3 cells that are ovarian cancer cells expressing high levels of MUC16_{high}.

FIG. 9B are graphs that show the binding of 21G6 anti-MUC16 clone to SKOV3 MUC16_{low} cells and MUC16(−) HEK293 cells.

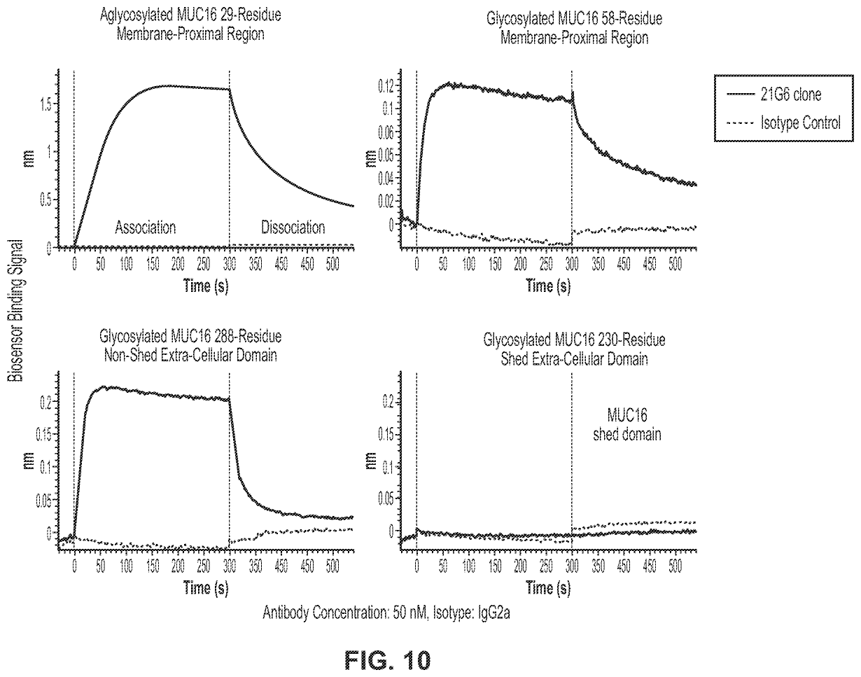

FIG. 10 are graphs that show that the 21G6 anti-MUC16 clone binds a membrane-proximal aglycosylated epitope and does not bind the MUC16 shed domain.

In some embodiments, provided herein is anti-MUC16 antibody, wherein the heavy chain variable domain (VH) of the antibody comprises the amino acid sequence of SEQ ID NO: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, or humanized versions thereof; and/or wherein the light chain variable domain (VL) of the antibody comprises the amino acid sequence of SEQ ID NOS: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, or humanized versions thereof, or sequences having at least 90, 91, 92, 93, 94, 95, 96, 97, 98, 99, or 100 percent identity to each.

In some embodiments, the scFv1 of each antibody arm comprises a first heavy chain variable domain (VH1) and a first light chain variable domain (VL1); and the scFv2 of each antibody arm comprises a first heavy chain variable domain (VH2) and a first light chain variable domain (VL2).

The scFvs on each antibody arm may be connected by a linker, e.g. a flexible linker. An exemplary linker comprises the following amino acid sequence: GGGGSGGGGSGGGGS (SEQ ID NO:375).

In some embodiments, the anti-MUC16 antibodies or binding fragments thereof provided herein are useful for the treatment of a disease or condition involving an immune response.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC. BC, or ABC, and if order is important in a particular context, also BA, CA. CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### Listing of Embodiments

Embodiment 1. An anti-MUC16 antibody or binding fragment thereof, wherein the antibody comprises:
a. a heavy chain variable domain (VH) complementarity determining region (CDR) 1 comprising the amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.
   Embodiment 2. The antibody of embodiment 1, wherein the antibody comprises:
a. a VH having at least 96, 97, 98, 99, or 100% sequence identity with the amino acid sequence of any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and
b. a VL having at least 96, 97, 98, 99, or 100% sequence identity with the amino acid sequence of any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.
   Embodiment 3. The antibody of any one of embodiments 1-2, wherein the antibody is a monoclonal antibody.
   Embodiment 4. The antibody of any one of embodiments 1-3, wherein the antibody is a full-length humanized antibody, chimeric antibody, a fusion protein, or an antibody fragment.
   Embodiment 5. The antibody of any one of embodiments 1-3, wherein the CDRs are selected from following three VH CDRs: SEQ ID NOS: 2, 3, 4; 12, 13, 14; 22, 23, 24; 32, 33, 34; 42, 43, 44; 52, 53, 54; 62, 63, 64; 72, 73, 74; 82, 83, 84; 92, 93, 94; 102, 103, 104; 112, 113, 114; 122, 123, 124; 132, 133, 134; 142, 143, 144; 152, 153, 154; 162, 163, 164; 172, 173, 174; 182, 183, 184; 192, 193, 194; 202, 203, 204; 212, 213, 214; 222, 223, 224; 232, 233, 234; 242, 243, 244; 252, 253, 254; 262, 263, 264; 272, 273, 274; 282, 283, 284; 292, 293, 294; 302, 303, 304; 312, 313, 314; 322, 323, 324; 332, 333, 334; 342, 343, 344; and 352, 353, 354; and comprises the amino acid sequences of the following three VL CDRs SEQ ID NOS: 7, 8, 9; 17, 18, 19; 27, 28, 29; 37, 38, 39; 47, 48, 49; 57, 58, 59; 67, 68, 69; 77, 78, 79; 87, 88, 89; 97, 98, 99; 107, 108, 109; 117, 118, 119; 127, 128, 129; 137, 138, 139; 147, 148, 149; 157, 158, 159; 167, 168, 169; 177, 178, 179; 187, 188, 189; 197, 198, 199; 207, 208, 209; 217, 218, 219; 227, 228, 229; 237, 238, 239; 247, 248, 249; 257, 258, 259; 267, 268, 269; 277, 278, 279; 287, 288, 289; 297, 298, 299; 307, 308, 309; 317, 318, 319; 327, 328, 329; 337, 338, 339; 347, 348, 349; and 357, 358, 359.
   Embodiment 6. The antibody fragment of embodiment 5, wherein the antibody binding fragment is fused to an Fc domain of any one of the following: human IgG1, human IgG2, human IgG3, and human IgG4.
   Embodiment 7. A method of treating a disease in a subject in need thereof, comprising administering to the subject a therapeutically effective amount of the antibody of any one of claims 1-6.
   Embodiment 8. A method of embodiment 7, where the disease is an autoimmune disease.
   Embodiment 9. A method of embodiment 7, where the disease is an inflammatory disease.
   Embodiment 10. The method of any one of embodiments 7-9, wherein the subject is human.
   Embodiment 11. An anti-MUC16 antibody that comprises an scFv1 and an scFv2 binding site in tandem on each antibody arm and wherein said scFv1 and scFv2 are linked by a linker, optionally a flexible linker.
   Embodiment 12. The antibody of embodiment 11, wherein the antibody has a total of four scFv binding sites in a single scFv-Fc formatted antibody.
   Embodiment 13. The antibody of embodiment 12, wherein the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2).
   Embodiment 14. The antibody of embodiment 13, wherein the VH1 region and the VH2 region each comprises the amino acid sequence of a heavy chain variable domain (VH) complementarity determining region (CDR) comprising the amino acid sequence of:
a. a heavy chain variable domain (VH) complementarity determining region (CDR) 1 comprising the amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.
   Embodiment 15. The antibody of embodiment 14, wherein the antibody comprises the amino acid sequences:
a. a VH comprising the amino acid sequence having at least 96, 97, 98, 99, or 100% sequence identity with any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and
b. a VL comprising the amino acid sequence having at least 96, 97, 98, 99, or 100% sequence identity with any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.
   Embodiment 16. A method of treating a disease in a subject in need thereof, comprising administering to the subject a therapeutically effective amount of the antibody of any one of claims 1-15.
   Embodiment 17. The method of embodiment 16, where the disease is a cancer that expresses MUC16.
   Embodiment 18. The method of embodiment 16, wherein the cancer is selected from ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16.
   Embodiment 19. The method of any one of embodiments 16-18, wherein the subject is human.
   Embodiment 20. A nucleic acid that expresses an anti-MUC16 antibody or binding fragment thereof, wherein the antibody comprises:
a. a nucleic acid having at least 95% sequence identity to a heavy chain variable domain that encodes a heavy chain variable domain (VH) comprising complementarity determining region (CDR) 1 of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a nucleic acid having at least 95% sequence identity to a heavy chain variable domain that encodes a heavy chain variable domain (VH) comprising a CDR2 of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a nucleic acid having at least 95% sequence identity to a heavy chain variable domain that encodes a heavy chain variable domain (VH) comprising a VH CDR3 comprising any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a nucleic acid having at least 95% sequence identity to a light chain variable domain that encodes a light chain variable domain (VL) comprising a VL CDR1 comprising any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a nucleic acid having at least 95% sequence identity to a light chain variable domain that encodes a light chain variable domain (VL) comprising a VL CDR2 comprising any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a nucleic acid having at least 95% sequence identity to a light chain variable domain that encodes a light chain variable domain (VL) comprising a VL CDR3 comprising any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.
   Embodiment 21. The nucleic acid of embodiment 20, wherein the VH is a nucleic acid having at least 96, 97, 98, 99, or 100% sequence identity with SEQ ID NO: 5; 15; 25; 35; 45; 55; 65; 75; 85; 95; 105; 115; 125; 135; 145; 155; 165; 175; 185; 195; 205; 215; 225; 235; 245; 255; 265; 275; 285; 295; 305; 315; 325; 335; 345; or 355, and the VL is a nucleic acid having at least 95, 96, 97, 98, 99, or 100% sequence identity with SEQ ID NO: 10; 20; 30; 40; 50; 60; 70; 80; 90; 100; 110; 120; 130; 140; 150; 160; 170; 180; 190; 200; 210; 220; 230; 240; 250; 260; 270; 280; 290; 300; 310; 320; 330; 340; 350, or a humanized version thereof.
   Embodiment 22. A vector that comprises the nucleic acid of any one of embodiments 20-21.
   Embodiment 23. A host cell that comprises the vector of embodiment 22.

What is claimed is:

1. An anti-MUC16 antibody or binding fragment thereof, wherein the antibody comprises:
a. a heavy chain variable domain (VH) complementarity determining region (CDR) 1 comprising an amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.

2. The antibody of claim 1, wherein the antibody comprises:
a. a VH having at least 96, 97, 98, 99, or 100% sequence identity with the amino acid sequence of any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and
b. a VL having at least 96, 97, 98, 99, or 100% sequence identity with the amino acid sequence of any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.

3. The antibody of claim 1, wherein the antibody is a monoclonal antibody.

4. The antibody of claim 1, wherein the antibody is a full-length humanized antibody, chimeric antibody, a fusion protein, or an antibody fragment.

5. The antibody of claim 1, wherein the CDRs are selected from following three VH CDRs: SEQ ID NOS: 2, 3, 4; 12, 13, 14; 22, 23, 24; 32, 33, 34; 42, 43, 44; 52, 53, 54; 62, 63, 64; 72, 73, 74; 82, 83, 84; 92, 93, 94; 102, 103, 104; 112, 113, 114; 122, 123, 124; 132, 133, 134; 142, 143, 144; 152, 153, 154; 162, 163, 164; 172, 173, 174; 182, 183, 184; 192, 193, 194; 202, 203, 204; 212, 213, 214; 222, 223, 224; 232, 233, 234; 242, 243, 244; 252, 253, 254; 262, 263, 264; 272, 273, 274; 282, 283, 284; 292, 293, 294; 302, 303, 304; 312, 313, 314; 322, 323, 324; 332, 333, 334; 342, 343, 344; and 352, 353, 354; and comprises the amino acid sequences of the following three VL CDRs SEQ ID NOS: 7, 8, 9; 17, 18, 19; 27, 28, 29; 37, 38, 39; 47, 48, 49; 57, 58, 59; 67, 68, 69; 77, 78, 79; 87, 88, 89; 97, 98, 99; 107, 108, 109; 117, 118, 119; 127, 128, 129; 137, 138, 139; 147, 148, 149; 157, 158, 159; 167, 168, 169; 177, 178, 179; 187, 188, 189; 197, 198, 199; 207, 208, 209; 217, 218, 219; 227, 228, 229; 237, 238, 239; 247, 248, 249; 257, 258, 259; 267, 268, 269; 277, 278, 279; 287, 288, 289; 297, 298, 299; 307, 308, 309; 317, 318, 319; 327, 328, 329; 337, 338, 339; 347, 348, 349; and 357, 358, 359.

6. The antibody of claim 1, wherein the antibody binding fragment is fused to an Fc domain of any one of the following: human IgG1, human IgG2, human IgG3, and human IgG4.

7. A method of treating a disease in a subject in need thereof, comprising administering to the subject a therapeutically effective amount of the antibody of claim 1.

8. The method of claim 7, where the disease is an autoimmune disease or an inflammatory disease.

9. The method of claim 7, wherein the subject is human.

10. An anti-MUC16 antibody that comprises an scFv1 and an scFv2 binding site in tandem on each antibody arm and wherein said scFv1 and scFv2 are linked by a linker, optionally a flexible linker.

11. The antibody of claim 10, wherein the antibody has a total of four scFv binding sites in a single scFv-Fc formatted antibody.

12. The antibody of claim 10, wherein the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2).

13. The antibody of claim 12, wherein the VH1 region and the VH2 region each comprises the amino acid sequence of a heavy chain variable domain (VH) complementarity determining region (CDR) comprising the amino acid sequence of:
a. a heavy chain variable domain (VH) complementarity determining region (CDR) 1 comprising the amino acid sequence of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a VH CDR2 comprising the amino acid sequence of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a VH CDR3 comprising the amino acid sequence of any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a light chain variable domain (VL) CDR1 comprising the amino acid sequence of any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a VL CDR2 comprising the amino acid sequence of any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a VL CDR3 comprising the amino acid sequence of any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.

14. The antibody of claim 11, wherein the antibody comprises the amino acid sequences:
a. a VH comprising the amino acid sequence having at least 96, 97, 98, 99, or 100% sequence identity with any one of the following SEQ ID NOs: 1; 11; 21; 31; 41; 51; 61; 71; 81; 91; 101; 111; 121; 131; 141; 151; 161; 171; 181; 191; 201; 211; 221; 231; 241; 251; 261; 271; 281; 291; 301; 311; 321; 331; 341; 351, 361, 363, or 365; and
b. a VL comprising the amino acid sequence having at least 96, 97, 98, 99, or 100% sequence identity with any one of the following SEQ ID NOs: 6; 16; 26; 36; 46; 56; 66; 76; 86; 96; 106; 116; 126; 136; 146; 156; 166; 176; 186; 196; 206; 216; 226; 236; 246; 256; 266; 276; 286; 296; 306; 316; 326; 336; 346; 356, 367, 369, or 371.

15. A method of treating a disease in a subject in need thereof, comprising administering to the subject a therapeutically effective amount of the antibody of claim 1.

16. The method of claim 15, where the disease is a cancer that expresses MUC16.

17. The method of claim 16, wherein the cancer is selected from ovarian cancer cells, breast cancer cells, prostate cancer cells, colon cancer cells, lung cancer cells, brain cancer cells, pancreatic cancer cells, kidney cancer cells, fallopian tube cancer cells, uterine (e.g., endometrial) cancer cells, primary peritoneum cancer cells or cancer cells of any other tissue that expresses MUC16.

18. The method of claim 15, wherein the subject is human.

19. A nucleic acid that expresses an anti-MUC16 antibody or binding fragment thereof, wherein the antibody comprises:
a. a nucleic acid having at least 95% sequence identity to a heavy chain variable domain that encodes a heavy chain variable domain (VH) comprising complementarity determining region (CDR) 1 of any one of the following SEQ ID NOs: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; 312; 322; 332; 342; or 352; and
b. a nucleic acid having at least 95% sequence identity to a heavy chain variable domain that encodes a heavy chain variable domain (VH) comprising a CDR2 of any one of the following SEQ ID NOs: 3; 13; 23; 33; 43; 53; 63; 73; 83; 93; 103; 113; 123; 133; 143; 153; 163; 173; 183; 193; 203; 213; 223; 233; 243; 253; 263; 273; 283; 293; 303; 313; 323; 333; 343; or 353; and
c. a nucleic acid having at least 95% sequence identity to a heavy chain variable domain that encodes a heavy chain variable domain (VH) comprising a VH CDR3 comprising any one of the following SEQ ID NOs: 4; 14; 24; 34; 44; 54; 64; 74; 84; 94; 104; 114; 124; 134; 144; 154; 164; 174; 184; 194; 204; 214; 224; 234; 244; 254; 264; 274; 284; 294; 304; 314; 324; 334; 344; or 354; and
d. a nucleic acid having at least 95% sequence identity to a light chain variable domain that encodes a light chain variable domain (VL) comprising a VL CDR1 comprising any one of the following SEQ ID NOs: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; 317; 327; 337; 347; or 357; and
e. a nucleic acid having at least 95% sequence identity to a light chain variable domain that encodes a light chain variable domain (VL) comprising a VL CDR2 comprising any one of the following SEQ ID NOs: 8; 18; 28; 38; 48; 58; 68; 78; 88; 98; 108; 118; 128; 138; 148; 158; 168; 178; 188; 198; 208; 218; 228; 238; 248; 258; 268; 278; 288; 298; 308; 318; 328; 338; 348; 358; and
f. a nucleic acid having at least 95% sequence identity to a light chain variable domain that encodes a light chain variable domain (VL) comprising a VL CDR3 comprising any one of the following SEQ ID NOs: 9; 19; 29; 39; 49; 59; 69; 79; 89; 99; 109; 119; 129; 139; 149; 159; 169; 179; 189; 199; 209; 219; 229; 239; 249; 259; 269; 279; 289; 299; 309; 319; 329; 339; 349; 359.

20. The nucleic acid of claim 19, wherein the VH is a nucleic acid having at least 96, 97, 98, 99, or 100% sequence identity with SEQ ID NO: 5; 15; 25; 35; 45; 55; 65; 75; 85; 95; 105; 115; 125; 135; 145; 155; 165; 175; 185; 195; 205; 215; 225; 235; 245; 255; 265; 275; 285; 295; 305; 315; 325; 335; 345; or 355, and the VL is a nucleic acid having at least 95, 96, 97, 98, 99, or 100% sequence identity with SEQ ID NO: 10; 20; 30; 40; 50; 60; 70; 80; 90; 100; 110; 120; 130; 140; 150; 160; 170; 180; 190; 200; 210; 220; 230; 240; 250; 260; 270; 280; 290; 300; 310; 320; 330; 340; 350, or a humanized version thereof.

21. A vector that comprises the nucleic acid of 19.

22. A host cell that comprises the vector of claim 21.
